(19) United States
(12) Patent Application Publication
(10) Pub. No.: US 2025/0206819 A1
(43) Pub. Date: Jun. 26, 2025

(54) DEIMMUNIZED ANTIBODIES SPECIFIC FOR CD3

(71) Applicant: MORPHOSYS AG, Planegg (DE)

(72) Inventors: Markus MOOSMEIER, Landau a. d. Isar - Thalham (DE); Andreas BÜLTMANN, Planegg (DE); Steffen RUNZ, Heidelberg (DE)

(21) Appl. No.: 18/847,483

(22) PCT Filed: Mar. 21, 2023
(86) PCT No.: PCT/EP2023/057248

§ 371 Date: Sep. 16, 2024

(30) Foreign Application Priority Data
Mar. 22, 2022 (EP) 22163663.2

(51) Int. Cl. C07K 16/28; A61P 37/04

(52) U.S. Cl. CPC C07K 16/2809; A61P 37/04; C07K 2317/31; C07K 2317/33; C07K 2317/55; C07K 2317/565; C07K 2317/622; C07K 2317/71; C07K 2317/92

(57) ABSTRACT

The present invention provides affinity optimized and deimmunized human antibodies that specifically bind to CD3. The present disclosure also provides bispecific antibodies comprising the optimized human antibodies e.g. for activating of T cells. The invention further relates to methods of generating the human antibodies and methods of using them in the treatment of diseases.

### FIELD OF THE INVENTION

The present invention relates to fully human antibodies that bind to CD3 including structurally modified variants therefrom having a reduced potential to bind to HLA proteins and thus having a reduced risk in electing an immune response in human beings once administered. The present invention further provides bispecific antibodies employing the CD3 specific antibodies disclosed herein, methods for producing such antibodies, and methods of using them in the treatment of diseases, such as to treat cancer.

### BACKGROUND

CD3 (cluster of differentiation 3) is a protein complex consisting of at least four invariant polypeptide chains, which are non-covalently associated with the T cell receptors (TCR) on the surface of T cells, typically referred to as the CD3 antigen complex. The CD3 antigen complex plays an important role in T cell activation upon antigen binding to the T cell receptor. CD3 has been extensively explored as a drug target. Therapeutic concepts based on bispecific antibodies targeting CD3 rely on simultaneous binding to cell surface antigens present on tumor cells and CD3 present on cytotoxic T cells with the aim that bound cytotoxic T cells directly kill the tumor cells (Miller and Kontermann, Bispecific antibodies for cancer immunotherapy: Current perspectives. BioDrugs 2010, 24(2):89-98).

One major problem of CD3 targeted therapies relies in their dose limiting toxicity, caused by off-target T cell activation. Such toxicities are basically driven by the inherent ability of CD3 antibodies to stimulate T cells irrespective of the presence of target cells. Indeed, many side effects observed in context of CD3 based antibody therapy appear to be associated with errant T cell function, such as the associated production of cytokines which may lead to a toxic cytokine release syndrome. Hence, in order to reduce such toxicities it is meanwhile well established to engage CD3 present on T cells in a monovalent fashion and by using antibody backbones lacking the ability to bind to Fc receptors on accessory cells, such as monocytes, B cells and NK cells. This approach avoids crosslinking of CD3 complexes present on T cells and their subsequent activation.

A plethora of antibodies binding to CD3 has been described in the art. This includes antibodies of rodent origin, such as OKT-3 (Kung P. et al., Science, 1979 Oct. 19; 206(4416):347-9) or SP34 (Yoshino N. et al., Exp. Anim 49:97-110, 2000; Conrad M L. et al., Cytometry 71A:925-33, 2007), as well as humanized derivatives of the same. In addition, an immense panel of de novo generated human or humanized antibodies with specificity for CD3 are described in the art. Because of their murine origin, SP34 or OKT-3 induce a strong human anti-mouse antibody (HAMA) immunogenic reaction in non-immunosuppressed humans, which limits their dosing potential and also can cause dangerous allergic reactions. In general, such immune responses require the uptake of a therapeutic (foreign) protein (i.e. therapeutic antibody) by antigen presenting cells (APCs). Once inside such cells, the protein is processed and released fragments of the protein (certain short peptide sequences form a complex with MHC class II molecules) are presented on the cell surface. Should such a complex be recognized by binding of the T cell receptor present on T cells, such cells can be activated to produce stimulatory cytokines. The cytokines will elicit differentiation of B-cells to mature antibody producing cells. In addition, such T cell responses may also mediate other deleterious effects on the patient such as inflammation and possible allergic reaction. It is however understood that certain peptides which are found to bind to MHC class II molecules are recognized as “self” within the organism into which a final protein is administered and as such do not elicit an immune response. Such peptides are found for example in germline human immunoglobulin variable region protein sequences.

Accordingly, humanized CD3 specific antibodies having reduced immunogenicity in humans were designed. A common aspect of the humanization process is the introduction of significant portions of amino acid sequences identical to that present in human antibody proteins and as such involves for instance the grafting of the CDRs of a non-human antibody into the closest germline human acceptor antibody framework. A significant drawback associated with the humanization process is a significant reduction in the binding affinity of the resulting humanized antibody as well as poor productibility and stability. Restoring the original properties is typically achieved by an iterative process of back mutating human residues with the amino acids at the same position in the non-human donor antibody. However, such back mutations may again increase the risk for induction of an immunogenic reaction in human beings.

The use of human antibodies therefore seems to be the ideal solution to overcome the aforementioned shortcomings of using murine or humanized antibodies, because immune responses in general are not mounted to autologous circulating proteins, such as immunoglobulins. But still human antibodies may provoke an immune response or be immunogenic when administered to certain individuals. For instance, recombinant human antibodies selected from human phage display libraries may still employ non-germline encoded protein sequences, such as consensus framework sequences derived from human framework sequence analysis, which may provoke an immune reaction. In addition, the introduced variability in the CDR regions (in particular during affinity maturation of initially selected antibodies) may give rise to an immune reaction. Such selected CDR amino acid sequences may have similarity to foreign proteins and as such may provide for epitopes considered as not generally available to the immune system.

As outlined above, for CD3 targeting therapeutics, avoidance of any unwanted T cell activation is of upmost importance. An immune reaction against a therapeutic CD3 specific antibody may restore bivalent or multivalent CD3 binding and unwanted cross-linking of CD3 complexes on T-cells and thus their activation Accordingly, the present invention primarily takes a new approach of providing optimized fully human antibodies specific for CD3 characterized by the removal of potential T cell epitopes present in the CDR regions of their parental fully human antibody counterpart and thus have a reduced risk in electing an immune response in human beings once administered.

### SUMMARY OF THE INVENTION

The present disclosure provides affinity optimized human CD3 specific antibodies and deimmunized variants thereof. The affinity optimized antibodies were altered in their complementary determine region (CDR) to reduce their immunogenicity in human beings or to reduce their risk in electing an immune response in human beings once administered.

In an embodiment, the present disclosure provides an isolated human antibody or antigen-binding fragment thereof specific for cluster of differentiation 3 (CD3), comprising
i. a heavy chain variable region (VH) comprising
   (a) a heavy chain complementary determining region (HCDR)1 comprising the amino acid sequence of GFSFGSHYMS (SEQ ID NO: 1),
   (b) a HCDR2 comprising the amino acid sequence of NINQIGYSSYYVESVKG (SEQ ID NO: 2), NINQIGYSSYYGESVKG (SEQ ID NO: 3) or NINQIGYSSYYEESVKG (SEQ ID NO: 4), and
   (c) a HCDR3 comprising the amino acid sequence of GYSAEFAHRSGLDV (SEQ ID NO: 5), GYSDEFATRSGLDV (SEQ ID NO: 6), GYSEEFAHRSGLDV (SEQ ID NO: 7), GYSDEFAKRSGLDV (SEQ ID NO: 8) or GYSDEFAHRSGLDV (SEQ ID NO: 9), and
ii. a variable light chain region (VL) comprising
   (a) a light chain complementary determining region (LCDR)1 comprising the amino acid sequence of SGSSSNIGSNYVY (SEQ ID NO: 10),
   (b) a LCDR2 comprising the amino acid sequence of RNNQRPS (SEQ ID NO: 11), and
   (c) a LCDR3 comprising the amino acid sequence of AGWSRSLHGAV (SEQ ID NO: 12) or AGWSRELHGAV (SEQ ID NO: 13).

In an embodiment, the present disclosure provides an isolated human antibody or antigen-binding fragment thereof specific for CD3, wherein said antibody or antigen-binding fragment thereof cross-reactively binds to cynomolgus CD3.

In an embodiment, the present disclosure provides an isolated human antibody or antigen-binding fragment thereof specific for CD3, wherein said antibody or antigen-binding fragment is a deimmunized antibody.

In an embodiment, the present disclosure provides an isolated human antibody or antigen-binding fragment thereof specific for CD3, wherein said antibody or antigen-binding fragment thereof has a reduced risk in electing an immune response in human beings.

In an embodiment, the present disclosure provides an isolated human antibody or antigen-binding fragment thereof specific for CD3, wherein said antibody or antigen-binding fragment thereof comprises a VH comprising an amino acid sequence that is at least about 95%, 96%, 97%, 98%, 99% or 100% identical to an amino acid sequence selected from the group consisting of SEQ ID NO: 14, SEQ ID NO: 15, SEQ ID NO: 16, SEQ ID NO: 17, SEQ ID NO: 18 and SEQ ID NO: 19, and/or a VL comprising an amino acid sequence that is at least about 95%, 96%, 97%, 98%, 99% or 100% identical to the amino acid sequence of SEQ ID NO: 20 or SEQ ID NO: 21.

In an embodiment, the present disclosure provides an isolated human antibody or antigen-binding fragment thereof specific for CD3, wherein the VH and VL are selected from the group consisting of:
i. the VH comprising the amino acid sequence of SEQ ID NO: 14 and the VL comprising the amino acid sequence of SEQ ID NO: 20,
ii. the VH comprising the amino acid sequence of SEQ ID NO: 15 and the VL comprising the amino acid sequence of SEQ ID NO: 21,
iii. the VH comprising the amino acid sequence of SEQ ID NO: 16 and the VL comprising the amino acid sequence of SEQ ID NO: 21,
iv. the VH comprising the amino acid sequence of SEQ ID NO: 17 and the VL comprising the amino acid sequence of SEQ ID NO: 21,
v. the VH comprising the amino acid sequence of SEQ ID NO: 18 and the VL comprising the amino acid sequence of SEQ ID NO: 21, and
vi. the VH comprising the amino acid sequence of SEQ ID NO: 19 and the VL comprising the amino acid sequence of SEQ ID NO: 21.

In an embodiment of the present disclosure, said isolated human antibody or antigen-binding fragment thereof specific for CD3 is a recombinant antibody or antigen-binding fragment thereof. In an embodiment of the present disclosure, said isolated human antibody or antigen-binding fragment thereof specific for CD3 is a monoclonal antibody or antigen-binding fragment thereof.

In an embodiment of the present disclosure, said isolated antigen-binding fragment specific for CD3 is a Fab, Fab′, (Fab′)₂, Fv, or scFv. In an embodiment of the present disclosure, said isolated human antibody specific for CD3 antibody is a full-length antibody.

In an embodiment, the present disclosure provides a bispecific antibody comprising a first antigen-binding fragment of an isolated human antibody specific for CD3 according to the present disclosure and a second antigen-binding fragment of an antibody, which binds a different target antigen than said first antigen-binding fragment. In an embodiment of the present disclosure, said second antigen-binding fragment binds to a cell surface antigen, in particular a tumor associated cell surface antigen.

In an embodiment of the present disclosure, said isolated human antibody specific for CD3 according to the present disclosure or the bispecific antibody comprising the antigen-binding fragment of an isolated human antibody specific for CD3 according to the present disclosure comprises an Fc region comprising one or more amino acid substitutions that reduce binding to an Fc receptor and/or effector function.

In an embodiment, the present disclosure provides a nucleic acid composition comprising a nucleic acid sequence or a plurality of nucleic acid sequences encoding the isolated human antibody or antigen-binding fragment thereof specific for CD3 or the bispecific antibody comprising the antigen-binding fragment of an isolated human antibody specific for CD3 according to the present disclosure.

In an embodiment, the present disclosure provides a vector composition comprising a vector or a plurality of vectors comprising the nucleic acid sequence or plurality of nucleic acid sequences according to the present disclosure. In an embodiment, the present disclosure provides a host cell comprising the vector composition according to the present disclosure. In an aspect, said host cell is mammalian cell. In an aspect, said host cell is prokaryotic cell.

In an aspect, the present disclosure provides a method of producing an antibody or antigen-binding fragment thereof according to the present disclosure, comprising the steps of culturing the said host cell under conditions suitable for the expression of the antibody and optionally recovering said antibody or antigen-binding fragment thereof. In a further aspect, the present disclosure provides an antibody or antigen-binding fragment thereof produced by the methods described herein.

In an embodiment, the present disclosure provides a pharmaceutical composition comprising the isolated human antibody or antigen-binding fragment thereof specific for CD3 according to the present disclosure or the bispecific antibody comprising the antigen-binding fragment of an isolated human antibody specific for CD3 according to the present disclosure and a pharmaceutically acceptable carrier or excipient.

In an embodiment, the present disclosure provides an isolated human antibody or antigen-binding fragment thereof specific for CD3 or a pharmaceutical composition according to the present disclosure for use as a medicament.

There is utility in the claimed antibody or antigen-binding fragment thereof. Furthermore, there is utility in the claimed method to generate such antibody or antigen-binding fragment thereof. Utilization of the claimed antibody or antigen-binding fragment thereof is to target T cells expressing CD3, and for stimulating T cell activation, e.g., under circumstances where T cell-mediated killing is beneficial or desirable. In particular the claimed antibody or antigen-binding fragment thereof is for therapeutic use, such as the treatment of cancer.

### BRIEF DESCRIPTION OF THE DRAWINGS

FIG. 1: FIG. 1A depicts the basic structure of the bispecific 2+1 Fab₂-Fc-scFv antibody format of Example 1. The bispecific antibody format encompasses an aglycosylated monoclonal IgG1 backbone plus one additional scFv antibody, with the N-terminus of the VL of the scFv fused to the C-terminus of one IgG heavy chain via a peptide linker. To facilitate heterodimerization of the two different heavy chains, knob-into-hole mutations were introduced in both CH3-Fc domain. In addition to these mutations, the Fc region included amino acid substitutions that result in a Fc region that is unable to interact with IgG Fc receptors (Fc gamma receptors) and complement. In the present examples, both Fabs arms of the bispecific antibody bind to HER2 while the scFv is specific for CD3. FIG. 1B depicts the basic structure of the bispecific 1+1 Fab₂-Fc antibody format of Example 2. This bispecific antibody format reflects the typical Y-shape of a conventional IgG molecule with one Fab arm binds to a tumour target and the other Fab arm binds to CD3. Bispecific 1+1 Fab₂-Fc antibodies were generated in vitro by Fab-arm exchange. To enable the production of bispecific antibodies by this method, source IgG1 molecules carrying a single mutation in the CH3 domain were generated: in one source IgG1 antibody the F405L mutation (i.e. the CD3 specific antibody), in the other source IgG1 antibody the K409R mutation (i.e. the anti-HER antibody). In addition to these mutations, the source IgG1 antibodies included substitutions that result in a Fc region that is unable to interact with IgG Fc receptors (Fc gamma receptors) and complement.

FIG. 2: Basic structure of bispecific Fab₂-Fv-Fc antibody format of Example 3 and Example 4. This bispecific antibody format is built from an aglycosylated monoclonal human IgG1 antibody backbone plus one additional Fv fragment incorporated between the Fc region and the two Fab arms of the IgG1 backbone. In the present examples, both Fabs arms bind to HER2 while the “extra” Fv fragment comprises the variable regions of an antibody specific for CD3 according to the present disclosure. FIG. 2 also depicts the different improved peptide linkers used to connect the additional Fv fragment to the Fab arms and Fc region.

FIG. 3: Cell binding of mammalian produced bispecific antibodies with specify for HER2 and CD3 according to Example 2.2 comprising variable domains of affinity matured or cross-cloned CD3 specific antibodies according to the present disclosure. Shown is cell binding (signal over background) to CD3 positive Jurkat cells as a function of bispecific antibody concentration determined by flow cytometry.

FIG. 4: Cytotoxicity assay for mammalian produced bispecific antibodies with specify for HER2 and CD3 according to Example 2.4 comprising variable domains of affinity matured or cross-cloned CD3 specific antibodies of to the present disclosure on either HER2 expressing SKBR3 cells (FIG. 4A) or HER2 positive MCF-7 cells (FIG. 4B) in the presence of human T cells derived from one donor. Cytotoxic activity of human T cells is assessed by measuring incorporated CellToxGreen fluorescence. The graph shows the relative fluorescence levels of HER2 expressing SKBR3 or MCF-7 cells as a function of bispecific antibody concentration.

FIG. 5: T cell activation assay for mammalian produced bispecific antibodies with specify for HER2 and CD3 according to Example 2.5 comprising variable domains of affinity matured or cross-cloned CD3 specific antibodies according to the present disclosure. Activation of T cells is determined by evaluation of CD69 expression on CD8 positive T cells as assessed by flow cytometry. Shown is the percentage of CD69+ activated CD8+ T cells derived from 3 different donors as a function of bispecific antibody concentration.

FIG. 6: Cytotoxicity assay of mammalian produced bispecific antibodies with specify for HER2 and CD3 according to Example 3.6 comprising the variable domains of one cross-cloned CD3 specific antibody of Example 2 (CD3-MABopt-cc) and the two most potent Linker Combinations (Linker Combination 2 and Linker Combination 5) in reference to the originally disclosed Linker Combination P. Shown is killing of HER2 expressing SKBR3 cells in presence of human T cells derived from one donor. Cytotoxic activity of human T cells is assessed by measuring incorporated CellToxGreen fluorescence. The graph shows the relative fluorescence levels of HER2 expressing SKOV-3 cells as a function of bispecific antibody concentration.

FIG. 7: T cell activation assay for mammalian produced bispecific antibodies with specify for HER2 and CD3 according to Example 3.7 comprising the variable domains of one cross-cloned CD3 specific antibody of Example 2 (CD3-MABopt-cc) and 6 Linker Combinations (Linker Combinations 1-6) in reference to the originally disclosed Linker Combination P. Activation of T cells is determined by evaluation of CD69 expression on CD4+ T cells (FIG. 7A) or CD8+ T cells (FIG. 7B) as assessed by flow cytometry.

FIG. 7A is a graph showing the average percentage of CD69+ activated CD4+ T cells derived from 3 different donors as a function of bispecific antibody concentration. FIG. 7B is a graph showing the average percentage of CD69+ activated CD8+ T cells derived from 3 different donors as a function of bispecific antibody concentration.

FIG. 8: Epibase™ (Lonza, Epibase Version: v3.0) in silico screening results for the HCDR3 region of the cross-cloned antibody CD3-MABopt-cc from Example 2 for identification of potential T cell epitopes. Human antibody germline encoded sequence regions were excluded from analysis. For analysis, the whole VH sequence is splitted into overlapping 10mer peptides, each of which is shifted by one amino acid. The left panel of FIG. 8 lists the analyzed 10mer peptides starting from amino acid position 90 to position 112 on the VH of CD3-MABopt-cc spanning its entire HCDR3 region. Potential peptide/HLA binding for each analyzed 10mer peptide is determined for the HLA class II allotypes of the major Caucasian DRB1 alleles as shown in the upper panel/columns of FIG. 8. For each DRB1 allotype, its natural occurring frequency is provided (e.g. for DRB1*01:01: 15%). As per example, Peptide 93 is predicted to bind to 3 allotypes of the DRB1 alleles with moderate (M) and to 2 allotypes of the DRB1 alleles with strong (S) affinity. Such a peptide reflects a “T cell epitope” as defined herein. The provided Risk Score of 24.5 for this peptide is calculated as the sum of the natural occurring frequencies of the allotypes of the DRB1 alleles to which such peptide binds to (e.g. for Peptide 93: 24+6+12+6+5=53 (rounded), exact: 51.9). In sum, 10 T cell epitopes (Peptides 92, 93, 94, 95, 97, 100, 102, 104, 110, 112) and 2 Hotspots can be allocated to the HCDR3 region of CD3-MABopt-cc (the first Hotspots spans Peptides 92-97 and the second Hotspot spans peptides 110-112). Hotspots reflects an accumulation of neighboring T cells epitopes. Such Hotspots are identified based on an “4 over 3”, which means that at least 4 allotypes of the DRB1 alleles must bind with moderate or strong affinity to at least two of three consecutive analyzed 10mer peptides. In addition, at most one 10mer peptide, that is not identified as a T cell epitope, can be part of a Hotspot. Consequently, not every identified T cell epitope must be part of a Hotspot (see for instance peptides 100, 102 or 104 of FIG. 8). On the other hand, a peptide not identified as a T cell epitope may be still part of a Hotspot (see for instance Peptide 96 or Peptide 111 of FIG. 4). Each analyzed 10mer peptide being part of a Hotspot is defined as a H-line. An accumulated Risk Score for an Hotspot can be calculated as the sum of Risk Scores determined for each T cell epitope within a Hotspot and is defined herein as the “H-Score”. For instance, the 2^{nd} Hotspot of the HCDR3 consists of 3 H-Lines. The H-score is thus calculated by the sum of Risk Scores for each T cell epitope covered by this Hotspot, e.g. 27.8+60.3=88.

FIG. 9: Epibase™ in silico mutation analysis: Impact of in silico single amino acid substitutions (upper panel) for each HCDR3 position (Position 92-112) of CD3-MAB_{opt-cc} on number of T cell epitopes (left panel of FIG. 4) and corresponding Absolut Risk Score (right panel of FIG. 4) for the VH of CD3-MAB_{opt-cc}. Numbers with bold borders indicate amino acid substitutions which were selected for gene synthesis of respective VH variants of CD3-MAB_{opt-cc}. Amino acid substitutions which resulted in a decrease of T cell epitopes of 2 or greater and allowed of making conservative amino acid substitutions were preferably selected. In addition, care was taken not to introduce potential posttranslational modification sites (“PTM motifs”) into the CDR regions. As per example, for the HCDR3, 42 single amino acid substitutions (single point variants) were selected for gene synthesis.

FIG. 10: Summary of biophysical und functional properties of 27 preferred CDR single point variants of CD3-MAB_{opt-cc}. after characterization in the bispecific Fab₂-Fv-Fc antibody format of Example 4. For each substitution, the reduction of T cell epitopes in the VH or VL of CD3-MAB_{opt-cc} is shown, as well as monomer content and yield of purified bispecific antibodies preparations, ELISA binding and affinities on recombinant human CD3epsilon. The last column of FIG. 10 depicts variants selected for a combinatorial in silico mutation analysis. The first row (wt (parent.)) indicates the functional properties determined for the bispecific antibody comprising the variable domains of antibody CD3-MAB_{opt-cc}.

FIG. 11: Epibase™ in silico combinatorial mutation analysis. Exemplary results for 54 combinatorial amino acid substitutions in the HCDR1-3 regions of CD3-MAB_{opt-cc} which resulted in a reduction of 3 Hotspots. The first row of the shown table indicates risk parameters (Absolut Score, Hotspots, Absolut H-Score and Absolut H-lines) for the VH of CD3-MAB_{opt-cc}. For each combined variant, the reduction of each risk parameter in reference (as a delta value) to the determined risk parameters of the VH of CD3-MABopt. is provided.

FIG. 12: Summary of the biophysical und functional properties of 33 preferred combined variants of CD3-MAB_{opt-cc}. after characterization in the bispecific Fab₂-Fv-Fc antibody format of Example 4. The first row indicates functional properties determined for the bispecific antibody comprising the VH and VL of CD3-MAB_{opt-cc}. For each combined variant, the reduction of each risk parameter (Absolut Score, Hotspots, Absolut H-Score and Absolut H-lines) in reference (as a delta value) to the determined risk parameters of the VH of CD3-MAB_{opt-cc} is provided. In addition, monomer content of purified bispecific antibody preparations, ELISA binding and affinities on recombinant human CD3epsilon antigen as well as functional activity in a receptor gene assays on SKOV-3 cells at two bispecific antibody concentrations are shown.

FIG. 13: Cytotoxicity assay according Example 4.12 for the mammalian produced bispecific antibody BissIg_21_CD3-MABdeimm_3 for one donor comprising the most preferred deimmunized combined variant VH and VL sequences of CD3-MABdeimm_3 in reference to the bispecific antibody BissIg_21_CD3-MABopt_cc comprising the unaltered CD3 specific variable domains of CD3-MABopt_cc. Cytotoxic activity of human T cells is assessed by measuring incorporated CellToxGreen fluorescence. The graph shows the relative fluorescence levels of HER2 expressing SKOV-3 cells as a function of bispecific antibody concentration.

FIG. 14: Exemplary T cell activation assay according Example 4.13 assay for the bispecific antibodies BissIg_21_CD3-MABdeimm_1, BissIg_21_CD3-MABdeimm_2, BissIg_21_CD3-MABdeimm_3, BissIg_21_CD3-MABdeimm_4, BissIg_21_CD3-MABdeimm_5 encompassing the 5 most preferred deimmunized combined variant VH and VL sequences in reference to the bispecific antibody BissIg_21_CD3-MABopt_cc comprising the unaltered CD3 specific variable domains of CD3-MABopt_cc. As positive control, results for the murine CD3 specific OKT-3 IgG are shown. Activation of T cells is determined by evaluation of CD69 expression on CD4+ T cells (FIG. 14A) and CD8+ T cells (FIG. 14B) as assessed by flow cytometry. Shown is average percentage of CD69+ activated CD4+ T cells or CD8+ T cells, respectively derived from one donors as a function of bispecific antibody concentration.

### DETAILED DESCRIPTION OF THE DISCLOSURE

### Definitions

“CD3” refers to an antigen which is expressed on T cells as part of the multimolecular T cell receptor (TCR) and which consists of a homodimer or heterodimer formed from the association of two of four receptor chains: CD3epsilon (CD3epsilon), CD3delta, CD3zeta, and CD3gamma.

Human CD3epsilon (or human CD3e) has the amino acid sequence of UniProt P07766:

(SEQ ID NO: 45)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI
(signal sequence underlined, intracellular region
italic, transmembrane region bold).

The mature extracellular domain of human CD3epsilon without signal sequence comprises amino acid residues 22-126 and has the amino acid sequence of:

(SEQ ID NO: 46)
QDGNEEMGGITQTPYKVSISGTTVILTCPQYPGSEILWQHNDKNIGGDED
DKNIGSDEDHLSLKEFSELEQSGYYVCYPRGSKPEDANFYLYLRARVCEN
CMEMD

Cynomolgus CD3epsilon (or cyno CD3e) has the amino acid sequence of UniProt Q9515

(SEQ ID NO: 47)
MQSGTRWRVLGLCLLSIGVWGQDGNEEMGSITQTPYQVSISGTTVILTCS
QHLGSEAQWQHNGKNKEDSGDRLFLPEFSEMEQSGYYVCYPRGSNPEDAS
HHLYLKARVCENCMEMDVMAVATIVIVDICITLGLLLLVYYWSKNRKAKA
KPVTRGAGAGGRQRGQNKERPPPVPNPDYEPIRKGQQDLYSGLNQRRI
(signal sequence underlined, intracellular region
italic, transmembrane region bold).

The mature extracellular region of cynomolgus monkey CD3 epsilon without the signal sequence comprises amino acid residues 22-198 and has the amino acid sequence of:

(SEQ ID NO: 48)
QDGNEEMGSITQTPYQVSISGTTVILTCSQHLGSEAQWQHNGKNKEDSG
DRLFLPEFSEMEQSGYYVCYPRGSNPEDASHHLYLKARVCENCMEMDVM
AVATIVIVDICITLGLLLLVYYWSKNRKAKAKPVTRGAGAGGRQRGQNK
ERPPPVPNPDYEPIRKGQQDLYSGLNQRRI.

The term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “antigen” or “target antigen” as used herein refers to any molecule of interest that can be bound by one of the binding sites present in an antibody. Typically, an antigen is a peptide, a protein or any other proteinaceous molecule. Alternatively, an antigen may be any other organic or inorganic molecule, such as carbohydrate, fatty acid, lipid, dye or flourophor.

The term “antibody” as used herein refers to a protein comprising at least two heavy (H) chains and two light (L) chains interconnected by disulfide bonds which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelid antibodies and chimeric antibodies.

The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and AI-Lazikani et al., (1997) J. Mol. Biol. 273:927-948. The term “antibody” as used herein is intended to include monospecific specific antibodies as well as bispecific and multispecific antibodies.

The term “antibody fragment” or “antigen-binding fragment” of an antibody, as used herein, refers to one or more portions of an antibody that retain the ability to specifically interact with (e.g., by binding, steric hindrance, stabilizing spatial distribution) an antigen. Examples of antibody fragments or antigen-binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment” or “antigen-binding fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments or antigen-binding fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The term “Fc region” as used herein refers to the two Fc region subunits being capable of stable association with each other thus forming the dimeric C-terminal region of an immunoglobulin. Accordingly, the two Fc region subunits (e.g. the first the second Fc region subunit) are complementary to each other. The Fc region of a regular IgG molecule exists as a dimer, each subunit of which comprises the CH2 and CH3 IgG heavy chain constant domains.

A “Fc region subunit” as used herein refers to one of the two polypeptides forming the dimeric Fc region of an immunoglobulin, i.e. a polypeptide comprising the C-terminal constant regions of an immunoglobulin heavy chain, capable of stable self-association. Accordingly, the two Fc region subunits ((.g. the first the second Fc region subunit) which form the dimeric Fc region are complementary to each other. For example, IgG Fc region subunit comprises an IgG CH2 and an IgG CH3 constant domain. The term includes native sequence Fc region subunits and variant Fc region subunits. Although the boundaries of the Fc region subunits of an IgG heavy chain might vary slightly, the human IgG heavy chain Fc region subunit is usually defined to extend from Cys226, or from Pro230, to the C-terminus of the heavy chain. However, the C-terminal lysine (Lys447) of the Fc region subunit may or may not be present. Unless otherwise specified herein, numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, MD, 1991.

A “human antibody” or “human antibody fragment” or “human antigen-binding fragment”, as used herein, includes antibodies and antibody fragments having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86). Thereby said human antibody can be obtained from technology platforms which comprise antibodies derived from human germline genes either generated by PCR-amplification of VHA/L repertoire isolated from B-cells or are generated synthetically. Technology platforms include library based approaches comprising human immunoglobulin genes displayed on phage, ribosome or yeast. Respective display technologies are standard in the scientific community. Furthermore immunization of a transgenic mouse carrying human immunoglobulin repertoire is another approach to generate human antibodies against an antigen of interest. Antibodies or fragments thereof selected from an antibody library based on the MorphoSys HuCAL® concept (Knappik et al., (2000) J Mol Biol 296:57-86) or Ylanthia® concept library (Tiller et al. mAbs 5:3, 1-26; May/June (2013) and U.S. Pat. No. 8,728,981) are considered as fully human.

The term “isolated” refers to a compound, which can be e.g. an antibody or antibody fragment or antigen-binding fragment, that is substantially free of other antibodies or antibody fragments having different antigenic specificities. Thus, in some aspects, antibodies provided are isolated antibodies which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody”, as used herein, includes all antibodies or antigen-binding fragments that are prepared, expressed, created or segregated by means not existing in nature. For example antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody may be a monoclonal antibody.

As used herein, the term “monoclonal antibody” refers to an antibody that is derived from a single clone, including any eukaryotic, prokaryotic, or phage clone, and not the method by which it is produced. Monoclonal antibodies as disclosed herein may be made by the hybridoma method as described in Kohler et a/.; Nature, 256:495 (1975) or may be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Other exemplary methods of producing other monoclonal antibodies are provided in the Examples herein.

As used herein, an antibody “binds specifically to”, “specifically binds to”, is “specific to/for” or “specifically recognizes” an antigen if such antibody is able to discriminate between such antigen and one or more reference antigen(s), since binding specificity is not an absolute, but a relative property. For example, a standard ELISA assay can be carried out. The scoring may be carried out by standard colour development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

As used herein, “an antibody or antigen-binding fragment thereof that binds to CD3” or an “anti-CD3 antibody or antigen-binding fragment thereof” or an “antibody or antigen-binding fragment thereof specific for CD3” includes antibodies and antibody fragments thereof or antigen-binding fragments thereof that specifically recognize one or more CD3 subunits (e.g., epsilon), as well as antibodies and antibody fragments that specifically recognize a dimeric complex of two CD3 subunits (e.g. gamma/epsilon, delta/epsilon). The antibodies and antigen-binding fragments thereof of the present disclosure may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the term “cell surface” means one or more protein(s) that is/are expressed on the surface of a cell in vitro or in vivo, such that at least a portion of the protein is exposed to the extracellular side of the cell membrane and is accessible to an antigen-binding portion or fragment of an antibody. “Cell surface expressed CD3” includes CD3 proteins contained within the context of a functional T cell receptor in the membrane of a cell. The expression “cell surface expressed CD3” includes CD3 protein expressed as part of a homodimer or heterodimer on the surface of a cell (e.g., gamma/epsilon, delta/epsilon, and zeta/zeta CD3 dimers). The expression, “cell surface expressed CD3” also includes a CD3 chain (e.g., CD3epsilon) that is expressed by itself, without other CD3 chain types, on the surface of a cell. A “cell surface expressed CD3” can comprise or consist of a CD3 protein expressed on the surface of a cell which normally expresses CD3 protein. Alternatively, “cell surface expressed CD3” can comprise or consist of CD3 protein expressed on the surface of a cell that normally does not express human CD3 on its surface but has been artificially engineered to express CD3 on its surface.

The term “cross-reactively binds” or the term “is cross-reactive” are used herein interchangeably and refers to an antibody or antigen-binding fragment which has the ability to specifically bind to more than one antigen. For example, the antibody according to the present disclosure antibody cross-reactively binds to cynomolgus CD3, such as cynomolgus CD3epsilon.

As used herein, the term “affinity” refers to the strength of interaction between the antibody and its target at a single site. Within each site, the binding region of the antibody interacts through weak non-covalent forces with its target at numerous sites; the more interactions, the stronger the affinity.

The term “K_{D}”, as used herein, refers to the dissociation constant, which is obtained from the ratio of K_{d} to Kₐ (i.e. K_{d}/Kₐ) and is expressed as a molar concentration (M). K_{D} values for antigen binding moieties like e.g. monoclonal antibodies can be determined using methods well established in the art. Methods for determining the K_{D} of an antigen binding moiety like e.g. a monoclonal antibody are SET (soluble equilibrium titration) or surface plasmon resonance using a biosensor system such as a Biacore® system. In the present disclosure an antibody specific to the CD3 epsilon polypeptide typically has a dissociation rate constant (K_{D}) (k_{off}/kₒₙ) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower.

Compositions of the present disclosure may be used for therapeutic or prophylactic applications. The present disclosure, therefore, includes a pharmaceutical composition containing an antibody (or antigen-binding fragment thereof) as disclosed herein and a pharmaceutically acceptable carrier or excipient therefor. In a related aspect, the present disclosure provides a method for treating cancer. Such method contains the steps of administering to a subject in need thereof an effective amount of the pharmaceutical composition that contains an antibody (or antigen-binding fragment thereof) as described herein. The present disclosure provides therapeutic methods comprising the administration of a therapeutically effective amount of a human antibody or antigen-binding fragment thereof specific for CD3 as disclosed herein to a subject in need of such treatment. A “therapeutically effective amount” or “effective amount”, as used herein, refers to the amount of an CD3 specific antibody necessary to elicit the desired biological response. In accordance with the disclosure, the therapeutic effective amount is the amount of a CD3 specific antibody or antigen-binding fragment thereof necessary to treat and/or prevent a disease.

The terms “cell proliferative disease” or “proliferative disease” refer to a disease that is associated with some degree of abnormal cell proliferation. In one embodiment, the cell proliferative disease is cancer. In one embodiment, the cell proliferative disease is a tumor. The terms “cancer” and “cancerous” refer to or describe the physiological condition in mammals that is typically characterized by unregulated cell growth.

The term “tumor associated antigen” refers to an antigen that is expressed or present on the surface of a tumor or of a cell of the tumor stroma.

By “increase” is meant the ability to cause an overall increase, for example, of 20% or greater, of 50% or greater, or of 75%, 85%, 90%, 95%, or greater.

The term “EC50”, as used herein, refers to the concentration of an antibody or an antibody fragment or an bispecific antibody which induces a response in an assays half way between the baseline and maximum. It represents the antibody concentration at which 50% of the maximal effect is observed.

The term “IC50”, as used herein, refers to the concentration of an antibody or antibody fragment or bispecific antibody that inhibits a response in an assay half way between the maximal response and the baseline. It represents the antibody concentration that reduces a given response by 50%.

The terms “inhibition” or “inhibit” or “reduction” or “reduce” or “neutralization” or “neutralize” and the like refer to a decrease or cessation of any phenotypic characteristic (such as binding, a biological activity or function) or to the decrease or cessation in the incidence, degree, or likelihood of that characteristic. The “inhibition”, “reduction” or “neutralization” and the like needs not to be complete as long as it is detectable using an appropriate assay. In some aspects, by “reduce” or “inhibit” and the like is meant the ability to cause a decrease of 20% or greater. In another aspect, by “reduce” or “inhibit” is meant the ability to cause a decrease of 50% or greater. In yet another aspect, by “reduce” or “inhibit” and the like is meant the ability to cause an overall decrease of 75%, 85%, 90%, 95%, or greater.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

As used herein, “treatment”, “treat” or “treating” and the like refers to the clinical intervention in an attempt to alter the natural course of a disease in the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies or antigen-binding fragments thereof according to the preset disclosure are used to delay development of a disease or to slow the progression of a disease.

The term “multispecific” means that an antibody is able to specifically bind to two or more different antigens. Typically, a multispecific antibody comprises of two or more antigen-binding sites, each of which is specific for a different antigen or epitope.

The term “bispecific” means that an antibody is able to specifically bind to two different antigens. Typically, a bispecific antibody comprises two antigen-binding sites, each of which is specific for a different antigen or epitope.

As used herein, the terms “first” and “second” and the like are used for distinguishing when there is more than one of each type of component or type. Use of these terms is not intended to confer a specific order or orientation unless explicitly so stated.

As used herein, “amino acid residues” or “amino acid” will be indicated either by their full name or according to the standard three-letter or one-letter amino acid code. “Natural occurring amino acids” means the following amino acids:

TABLE 1
Natural occurring amino acids

| | Three letter | |
|---|---|---|
| Amino acid | code | One letter code |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | lle | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Effector function(s)” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Species”, as used in this context refers to any mammal, including rodents, such as mouse or rat, and primates, such as cynomolgus monkey (Macaca fascicularis), rhesus monkey (Macaca mulatta) or humans (Homo sapiens). Preferably the subject is a primate, most preferably a human.

“Deimmunization” refers to a method, wherein amino acids within an given antibody sequence that are predicted to bind effectively to HLA molecules are changed such that they no longer bind HLA and thus can no longer stimulate a T cell response. Accordingly, deimmunization renders a given protein or polypeptide non-immunogenic or less immunogenic to a given species. The elimination of T cell epitopes from proteins has been previously disclosed (see WO 98/52976). One suitable technique for deimmunizing antibodies is described, for example, WO 00/34317 or WO 2003/105058. For a therapeutic antibody, preferably all of the potential T cell epitopes are removed whilst retaining the functional activity of the unmodified parental antibody.

A “deimmunized” antibody as used herein refers to an antibody which was subject to deimmunization. A “deimmunized” antibody may be less or non-immunogenic in a given species compared to the unmodified parental antibody when used in vivo.

The term “T cell epitope” or “potential T cell epitope” or “MHC class II binding motif” as used herein refers to a specific 10mer peptide sequence within a given protein or polypeptide sequence which either binds with reasonable efficiency to MHC class II molecules or which in the form of peptide:MHC complexes bind strongly to the T cell receptors from the species to receive the therapeutic protein or which show the ability to stimulate T cells via presentation on MHC class II. Potential T cell epitopes can be measured by any computational or physical method to establish MHC binding.

The term “Hotspot” as used herein refers to a region within a given antibody heavy chain or light chain variable region with an accumulation of adjacent (predicted) T cell epitopes. A Hotspot according to the present disclosure is identified via a 4 over 3 algorithm as detailed in Figure legend 8 and Example 4.1 and requires that at least 4 allotypes of the DRB1 alleles bind to at least two of three consecutive analyzed 10mer peptides with moderate (M) or strong (S) affinity. In addition, at most one 10mer peptide, that is not identified as a T cell epitope, can be part of a Hotspot.

The term “Risk Score” as used herein provides a numerical value as a measure for the risk for a T cell epitope (analyzed 10mer peptide) to cause an immune reaction or immune response in a given population, e.g. a Caucasian population. The greater the value, the higher the risk. The Risk Score for a T cell epitope is calculated as the sum of the natural occurring population frequencies of the DRB1 alleles (HLA allotypes) to which such T cell epitope (10mer peptide) binds to. The Risk Score is determined by the computational method as described herein in Example 4, using the commercial available in silico screening tool: The Epibase™, Epibase Version: v3.0 (Lonza). Its basic method is described in WO 2003/105058.

The term “Absolut Risk Score” or “Score” as used herein provides a numerical value as a measure for the risk of an entire analyzed polypeptide to cause an immune reaction or immune response in a given population, e.g. a Caucasian population. The “Absolute Risk Score” is calculated as the sum of all individual Risk Scores determined for all T cell epitopes present in an analyzed polypeptide sequence, such as for an entire VH or VL sequence of an antibody.

“H-line” as used herein refers to one analyzed 10mer peptide sequence of an antibody heavy chain or light chain variable region identified as being part of an Hotspot.

“H-lines” as used herein provide a numerical value of the sum of analyzed 10mer peptide sequences of an an antibody heavy chain or light chain variable region, identified as being part of a Hotspot.

“Absolut H-lines” as used herein provides a numerical value of the sum of H-lines being part of all identified Hotspot in a polypeptide.

“H-score” as used herein provides a numerical value as a measure for the risk of one Hotspot to cause an immune reaction or immune response in a given human population, e.g. a Caucasian population. The H-score is calculated as the sum of the individual Risk Scores determined for of all T cell epitopes that are part of a Hotspot.

“Absolut H-score” as used herein provides a numerical value as a measure for the accumulated risk for all Hotspots identified in given antibody heavy chain or light chain variable region to cause an immune reaction or immune response in a given human population, e.g. a Caucasian population. The Absolute H-score is calculated as the sum of the Risk Scores determined for all T cell epitopes that are part of all identified Hotspots in a given antibody heavy chain or light chain variable region.

### EMBODIMENTS OF THE DISCLOSURE

Affinity optimized human antibodies specific for CD3 according the present disclosure are listed in Table 5 and Table 6. Deimmunized human antibodies specific for CD3 according the present disclosure are listed in Table 7. In an aspect, the present disclosure pertains to an isolated human antibody or antigen-binding fragment thereof specific for CD3 comprising 6 CDRs of any one of the antibodies listed in Tables 5-7. In one aspect, the disclosure pertains to an isolated human antibody or antigen-binding fragment thereof comprising a VH and a VL of any one of the antibodies listed in Tables 5-7.

In an embodiment, the present disclosure provides an isolated human antibody or antigen-binding fragment thereof specific for cluster of differentiation 3 (CD3), comprising
i. a heavy chain variable region (VH) comprising
   (a) a heavy chain complementary determining region (HCDR)1 comprising the amino acid sequence of GFSFGSHYMS (SEQ ID NO: 1),
   (b) a HCDR2 comprising the amino acid sequence of NINQIGYSSYYVESVKG (SEQ ID NO: 2), NINQIGYSSYYGESVKG (SEQ ID NO: 3) or NINQIGYSSYYEESVKG (SEQ ID NO: 4), and
   (c) a HCDR3 comprising the amino acid sequence of GYSAEFAHRSGLDV (SEQ ID NO: 5), GYSDEFATRSGLDV (SEQ ID NO: 6), GYSEEFAHRSGLDV (SEQ ID NO: 7), GYSDEFAKRSGLDV (SEQ ID NO: 8) or GYSDEFAHRSGLDV (SEQ ID NO: 9), and
ii. a variable light chain region (VL) comprising
   (d) a light chain complementary determining region (LCDR)1 comprising the amino acid sequence of SGSSSNIGSNYVY (SEQ ID NO: 10),
   (e) a LCDR2 comprising the amino acid sequence of RNNQRPS (SEQ ID NO: 11), and
   (f) a LCDR3 comprising the amino acid sequence of AGWSRSLHGAV (SEQ ID NO: 12) or AGWSRELHGAV (SEQ ID NO: 13).

In an embodiment, the present disclosure provides an isolated human antibody or antigen-binding fragment thereof specific for CD3, comprising a VH comprising
a) an HCDR1 region comprising the amino acid sequence of SEQ ID NO: 22,
b) an HCDR2 region comprising the amino acid sequence of SEQ ID NO: 23, and
c) an HCDR3 region comprising the amino acid sequence of SEQ ID NO: 5,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 12.

In an embodiment, the present disclosure provides an isolated human antibody or antigen-binding fragment thereof specific for CD3, comprising a VH comprising
a) an HCDR1 region comprising the amino acid sequence of SEQ ID NO: 1,
b) an HCDR2 region comprising the amino acid sequence of SEQ ID NO: 2, and
c) an HCDR3 region comprising the amino acid sequence of SEQ ID NO: 5,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 24.

In an embodiment, the present disclosure provides an isolated human antibody or antigen-binding fragment thereof specific for CD3, comprising a VH comprising
a) an HCDR1 region comprising the amino acid sequence of SEQ ID NO: 1,
b) an HCDR2 region comprising the amino acid sequence of SEQ ID NO: 2, and
c) an HCDR3 region comprising the amino acid sequence of SEQ ID NO: 5,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 12.

In an embodiment, the present disclosure provides an isolated human antibody or antigen-binding fragment thereof specific for CD3, comprising a VH comprising
a) an HCDR1 region comprising the amino acid sequence of SEQ ID NO: 1,
b) an HCDR2 region comprising the amino acid sequence of SEQ ID NO: 4, and
c) an HCDR3 region comprising the amino acid sequence of SEQ ID NO: 7,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 13.

In an embodiment, the present disclosure provides an isolated human antibody or antigen-binding fragment thereof specific for CD3, comprising a VH comprising
a) an HCDR1 region comprising the amino acid sequence of SEQ ID NO: 1,
b) an HCDR2 region comprising the amino acid sequence of SEQ ID NO: 3, and
c) an HCDR3 region comprising the amino acid sequence of SEQ ID NO: 9,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 13.

In an embodiment, the present disclosure provides an isolated human antibody or antigen-binding fragment thereof specific for CD3, comprising a VH comprising
a) an HCDR1 region comprising the amino acid sequence of SEQ ID NO: 1
b) an HCDR2 region comprising the amino acid sequence of SEQ ID NO: 3, and
c) an HCDR3 region comprising the amino acid sequence of SEQ ID NO: 6,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 13.

In an embodiment, the present disclosure provides an isolated human antibody or antigen-binding fragment thereof specific for CD3, comprising a VH comprising
a) an HCDR1 region comprising the amino acid sequence of SEQ ID NO: 1,
b) an HCDR2 region comprising the amino acid sequence of SEQ ID NO: 3, and
c) an HCDR3 region comprising the amino acid sequence of SEQ ID NO: 7,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 13.

In an embodiment, the present disclosure provides an isolated human antibody or antigen-binding fragment thereof specific for CD3, comprising a VH comprising
a) an HCDR1 region comprising the amino acid sequence of SEQ ID NO: 1
b) an HCDR2 region comprising the amino acid sequence of SEQ ID NO: 3, and
c) an HCDR3 region comprising the amino acid sequence of SEQ ID NO: 8,
   and a VL comprising
d) a LCDR1 region comprising the amino acid sequence of SEQ ID NO: 10,
e) a LCDR2 region comprising the amino acid sequence of SEQ ID NO: 11, and
f) a LCDR3 region comprising the amino acid sequence of SEQ ID NO: 13.

In an embodiment, the present disclosure provides an isolated human antibody or antigen-binding fragment thereof specific for cluster of differentiation 3 (CD3), comprising a heavy chain variable region (VH) comprising an amino acid sequence that is at least 95%, 96%, 97%, 98%, 99% or 100% identical to an amino acid sequence selected from the group consisting of SEQ ID NO: 14, SEQ ID NO: 15, SEQ ID NO: 16, SEQ ID NO: 17, SEQ ID NO: 18 and SEQ ID NO: 19. In an embodiment, the present disclosure provides an isolated human antibody or antigen-binding fragment thereof specific for cluster of differentiation 3 (CD3), comprising a light chain variable region (VL) comprising an amino acid sequence that is at least 95%, 96%, 97%, 98%, 99% or 100% identical to the amino acid sequence of SEQ ID NO: 20 or SEQ ID NO: 21. In an embodiment, the present disclosure provides an isolated human antibody or antigen-binding fragment thereof specific for cluster of differentiation 3 (CD3), comprising a heavy chain variable region (VH) comprising an amino acid sequence that is at least 95%, 96%, 97%, 98%, 99% or 100% identical to an amino acid sequence selected from the group consisting of SEQ ID NO: 14, SEQ ID NO: 15, SEQ ID NO: 16, SEQ ID NO: 17, SEQ ID NO: 18 and SEQ ID NO: 19 and a light chain variable region (VL) comprising an amino acid sequence that is at least 95%, 96%, 97%, 98%, 99% or 100% identical to the amino acid sequence of SEQ ID NO: 20 or SEQ 21. In an embodiment, the present disclosure provides an isolated human antibody or antigen-binding fragment thereof specific for cluster of differentiation 3 (CD3), comprising a heavy chain variable region (VH) comprising an amino acid sequence selected from the group consisting of SEQ ID NO: 14, SEQ ID NO: 15, SEQ ID NO: 16, SEQ ID NO: 17, SEQ ID NO: 18 and SEQ ID NO: 19. In an embodiment, the present disclosure provides an isolated human antibody or antigen-binding fragment thereof specific for cluster of differentiation 3 (CD3), comprising a light chain variable region (VL) comprising an amino acid sequence of SEQ ID NO: 20 or SEQ 21. In an embodiment, the present disclosure provides an isolated human antibody or antigen-binding fragment thereof specific for cluster of differentiation 3 (CD3), comprising a heavy chain variable region (VH) comprising an amino acid sequence selected from the group consisting of SEQ ID NO: 14, SEQ ID NO: 15, SEQ ID NO: 16, SEQ ID NO: 17, SEQ ID NO: 18 and SEQ ID NO: 19 and a light chain variable region (VL) comprising an amino acid sequence of SEQ ID NO: 20 or SEQ 21.

In an embodiment, the present disclosure provides an isolated human antibody or antigen-binding fragment thereof specific for cluster of differentiation 3 (CD3) comprising a heavy chain variable region (VH) and a light chain variable region (VL), wherein the VH and VL are selected from the group consisting of:
i. the VH comprising the amino acid sequence of SEQ ID NO: 25 and the VL comprising the amino acid sequence of SEQ ID NO: 20, and
ii. the VH comprising the amino acid sequence of SEQ ID NO: 14 and the VL comprising the amino acid sequence of SEQ ID NO: 26.

In an embodiment, the present disclosure provides an isolated human antibody or antigen-binding fragment thereof specific for cluster of differentiation 3 (CD3) comprising a heavy chain variable region (VH) and a light chain variable region (VL), wherein the VH and VL are selected from the group consisting of:
i. the VH comprising the amino acid sequence of SEQ ID NO: 14 and the VL comprising the amino acid sequence of SEQ ID NO: 20,
ii. the VH comprising the amino acid sequence of SEQ ID NO: 15 and the VL comprising the amino acid sequence of SEQ ID NO: 21,
iii. the VH comprising the amino acid sequence of SEQ ID NO: 16 and the VL comprising the amino acid sequence of SEQ ID NO: 21,
iv. the VH comprising the amino acid sequence of SEQ ID NO: 17 and the VL comprising the amino acid sequence of SEQ ID NO: 21,
v. the VH comprising the amino acid sequence of SEQ ID NO: 18 and the VL comprising the amino acid sequence of SEQ ID NO: 21, and
vi. the VH comprising the amino acid sequence of SEQ ID NO: 19 and the VL comprising the amino acid sequence of SEQ ID NO: 21.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 14 and a light chain variable region (VL) comprising the amino acid sequence of SEQ ID NO: 20.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 15 and a light chain variable region (VL) comprising the amino acid sequence of SEQ ID NO: 21.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 16 and a light chain variable region (VL) comprising the amino acid sequence of SEQ ID NO: 21.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 17 and a light chain variable region (VL) comprising the amino acid sequence of SEQ ID NO: 21.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 18 and a light chain variable region (VL) comprising the amino acid sequence of SEQ ID NO: 21.

In an embodiment, the present disclosure provides an isolated human antibody or antigen-binding fragment thereof specific for cluster of differentiation 3 (CD3) comprising a heavy chain variable region (VH) comprising the amino acid sequence of SEQ ID NO: 19 and a light chain variable region (VL) comprising the amino acid sequence of SEQ ID NO: 21.

In an aspect, said isolated human antibody or antigen-binding fragment thereof specific for CD3 is a monoclonal antibody or antigen-binding fragment. In another aspect, said isolated human antibody or antigen-binding fragment thereof is a recombinant antibody or antigen-binding fragment. In an aspect, said isolated human antibody or antigen-binding fragment thereof specific for CD3 is a synthetic antibody or antigen-binding fragment. In an aspect of the present disclosure, said antibody or antigen-binding fragment thereof specific for CD3 is a full-length IgG of an isotype selected from the group consisting of IgG1, IgG2, IgG3, and IgG4.

### Specificity

In an embodiment, the present disclosure provides an isolated human antibody or antigen-binding fragment thereof specific for cluster of differentiation 3 (CD3). In an aspect, the said isolated human antibody or antigen-binding fragment thereof specific for CD3 according to the present disclosure is specific for human CD3. In an aspect, the said isolated human antibody or and antigen-binding fragment thereof specific for CD3 according to the present disclosure is specific for cynomolgus CD3. In an aspect, said isolated human antibody or antigen-binding fragment thereof specific for CD3 according to the present disclosure is specific for human and cynomolgus CD3.

In an aspect of the present disclosure, the isolated human antibody or antigen-binding fragment thereof specific for CD3 according to the present disclosure is specific for human CD3 epsilon. In an aspect, the isolated human antibody or antigen-binding fragment thereof specific for CD3 according to the present disclosure is specific for cynomolgus CD3 epsilon. In an aspect, the isolated human antibody or antigen-binding fragment thereof specific for CD3 according to the present disclosure is specific for human and cynomolgus CD3 epsilon. In an aspect, the isolated human antibody or antigen-binding fragment thereof according to the present disclosure is specific for human and cynomolgus CD3 epsilon.

In an aspect, the present disclosure provides an isolated human antibody or antigen-binding fragment thereof specific for human CD3 according to the present disclosure, wherein said antibody or antigen-binding fragment thereof cross-reactively binds to cynomolgus CD3. In an aspect, the present disclosure provides an isolated human antibody or antigen-binding fragment thereof specific for human CD3 epsilon, wherein said antibody or antigen-binding fragment thereof cross-reactively binds to cynomolgus CD3 epsilon. In an aspect, the present disclosure provides an isolated human antibody or antigen-binding fragment thereof specific for CD3, wherein said antibody or antigen-binding fragment thereof specifically binds to human CD3 epsilon. In an aspect of the present disclosure, the isolated human antibody or antigen-binding fragment thereof specific for CD3 specifically binds to human and cynomolgus CD3 epsilon.

In an aspect, the present disclosure provides an isolated human antibody or antigen-binding fragment thereof specific for human CD3, wherein said human CD3 is human CD3 epsilon comprising the amino acid sequence of SEQ ID NO: 45 or SEQ ID NO: 46. In an aspect, the present disclosure provides an isolated human antibody or antigen-binding fragment thereof specific for human CD3, wherein said isolated human antibody or antigen-binding fragment thereof specifically binds to a human CD3 epsilon polypeptide comprising an amino acid sequence selected from the group consisting of SEQ ID NO: 45, SEQ ID NO: 46, SEQ ID NO: 41 and SEQ ID NO: 43.

In an aspect, the present disclosure provides an isolated human antibody or antigen-binding fragment thereof specific for human CD3, wherein said cynomolgus CD3 is cynomolgus CD3 epsilon comprising the amino acid sequence of SEQ ID NO: 47 or SEQ ID NO: 48. In an aspect, the present disclosure provides an isolated human antibody or antigen-binding fragment thereof specific for human CD3, wherein said isolated human antibody or antigen-binding fragment thereof specifically binds to a cynomolgus CD3 epsilon polypeptide comprising an amino acid sequence selected from the group consisting of SEQ ID NO: 47, SEQ ID NO: 48, SEQ ID NO: 42 and SEQ ID NO: 44.

In an aspect, the present disclosure provides an isolated human antibody or antigen-binding fragment thereof specific for human CD3, wherein said isolated human antibody or antigen-binding fragment thereof specifically binds to a polypeptide comprising the amino acid sequence selected from the group consisting of SEQ ID NO: 41, SEQ ID NO: 43, SEQ ID NO: 42, and SEQ ID NO: 44. In an aspect of the present disclosure, the isolated human antibody or antigen-binding fragment thereof specific for CD3 according to the present disclosure specifically binds to the extracellular region human CD3 epsilon.

In an aspect, the isolated human antibody or antigen-binding fragment thereof specific for CD3 according to the present disclosure specifically binds to the extracellular region human and cynomolgus CD3 epsilon. In an aspect of the present disclosure, said extracellular region of human CD3 epsilon comprises the amino acid sequence of SEQ ID NO: 46. In an further aspect, said extracellular region of cynomolgus CD3 epsilon comprises the amino acid sequence of SEQ ID NO: 48. In an aspect, the present disclosure pertains to an isolated human antibody or antigen-binding fragment thereof specific for a polypeptide comprising the amino acid sequence selected from the group consisting of: SEQ ID NO: 45, SEQ ID NO: 46, SEQ ID NO: 47, SEQ ID NO: 48, SEQ ID NO: 41, SEQ ID NO: 42, SEQ ID NO: 43, and SEQ ID NO: 44. In an aspect, the present disclosure pertains to an isolated human antibody or antigen-binding fragment thereof specific for CD3 according to the present disclosure which specifically binds to a polypeptide encoded by SEQ ID NO: 45, SEQ ID NO: 46, SEQ ID NO: 41, or SEQ ID NO: 43 and to a polypeptide encoded by SEQ ID NO: 47, SEQ ID NO: 48, SEQ ID NO: 42, or SEQ ID NO: 44.

In an aspect of the present disclosure, said isolated human antibody or antigen-binding fragment thereof specific for CD3 comprises
i. a heavy chain variable region (VH) comprising
   (a) a HCDR1 comprising the amino acid sequence of GFSFGSHYMS (SEQ ID NO: 1),
   (b) a HCDR2 comprising the amino acid sequence of NINQIGYSSYYVESVKG (SEQ ID NO: 2), NINQIGYSSYYGESVKG (SEQ ID NO: 3) or NINQIGYSSYYEESVKG (SEQ ID NO: 4), and
   (c) a HCDR3 comprising the amino acid sequence of GYSAEFAHRSGLDV (SEQ ID NO: 5), GYSDEFATRSGLDV (SEQ ID NO: 6), GYSEEFAHRSGLDV (SEQ ID NO: 7), GYSDEFAKRSGLDV (SEQ ID NO: 8) or GYSDEFAHRSGLDV (SEQ ID NO: 9), and
ii. a variable light chain region (VL) comprising
(d) a LCDR1 comprising the amino acid sequence of SGSSSNIGSNYVY (SEQ ID NO: 10),
(e) a LCDR2 comprising the amino acid sequence of RNNQRPS (SEQ ID NO: 11), and
(f) a LCDR3 comprising the amino acid sequence of AGWSRSLHGAV (SEQ ID NO: 12) or AGWSRELHGAV (SEQ ID NO: 13).

In an aspect of the present disclosure, said isolated human antibody or antigen-binding fragment thereof specific for CD3, comprises the VH and VL selected from the group consisting of:
i. the VH comprising the amino acid sequence of SEQ ID NO: 14 and the VL comprising the amino acid sequence of SEQ ID NO: 20,
ii. the VH comprising the amino acid sequence of SEQ ID NO: 15 and the VL comprising the amino acid sequence of SEQ ID NO: 21,
iii. the VH comprising the amino acid sequence of SEQ ID NO: 16 and the VL comprising the amino acid sequence of SEQ ID NO: 21,
iv. the VH comprising the amino acid sequence of SEQ ID NO: 17 and the VL comprising the amino acid sequence of SEQ ID NO: 21,
v. the VH comprising the amino acid sequence of SEQ ID NO: 18 and the VL comprising the amino acid sequence of SEQ ID NO: 21, and
vi. the VH comprising the amino acid sequence of SEQ ID NO: 19 and the VL comprising the amino acid sequence of SEQ ID NO: 21.

### Kinetics

In an aspect, the present disclosure pertains to an isolated human antibody or antigen-binding fragment thereof specific for human CD3, wherein said antibody or antigen-binding fragment thereof has a monovalent affinity for a human CD3 epsilon peptide comprising SEQ ID NO: 41 and/or SEQ ID NO: 43 with a lower K_{D} compared to the K_{D} of an antibody comprising a VH and VL comprising the amino acid sequence of SEQ ID NO: 25 and SEQ ID NO: 26, respectively.

In an aspect, the present disclosure pertains to an isolated human antibody or antigen-binding fragment thereof specific for human CD3, wherein said antibody or antigen-binding fragment thereof has a monovalent affinity for a human CD3 epsilon peptide comprising SEQ ID NO: 41 and/or SEQ ID NO: 43 with a K_{D} of 10 nM or less, such as 8 nM or less, 7 nM or less, 6 nM or less, 5 nM or less, 4 nM or less, 3 nM or less, 2 nM or less, 1 nM or less, 0.1 nM or less, 0.2 nM, or 0.1 nM less.

In an aspect, the present disclosure pertains to an isolated human antibody or antigen-binding fragment thereof specific for human CD3, wherein said antibody or antigen-binding fragment thereof has a monovalent affinity for a human CD3 epsilon peptide comprising SEQ ID NO: 41 or SEQ ID NO: 43 with a K_{D} of about 10 nM, about 9 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM.

In an aspect, the present disclosure pertains to an isolated human antibody or antigen-binding fragment thereof specific for CD3, wherein said antibody or antigen-binding fragment thereof has a monovalent affinity for a human CD3 epsilon polypeptide comprising SEQ ID NO: 41 or SEQ ID NO: 34 with a K_{D} between 0.1 nM and 10 nM.

In certain aspects, said monovalent affinity is determined for an scFv, Fv or Fab. In an aspect, said monovalent affinity is determined as described herein in Example 2.1, Example 3.4, Example 4.4 or Example 4.8. In an aspect, said monovalent affinity is determined in for antibody format as described herein in Example 2, Example 3 or Example 4.

In an aspect, said isolated human antibody or antigen-binding fragment thereof specific for cluster of differentiation 3 (CD3) comprises a heavy chain variable region (VH) comprising the amino acid sequence of SEQ ID NO: 15 and a light chain variable region (VL) comprising the amino acid sequence of SEQ ID NO: 21.

### Cell Binding

In an aspect, the present disclosure pertains to an isolated human antibody or antigen-binding fragment thereof specific for human CD3, wherein said antibody or antigen-binding fragment thereof specifically binds to human Jurkat cells (ATCC #TIB-152) with an EC₅₀ concentration between 40 nM and 120 nM, such as between 1 nM and 10 nM.

In an aspect, the present disclosure pertains to an isolated human antibody or antigen-binding fragment thereof specific for human CD3, wherein said antibody or antigen-binding fragment thereof specifically binds to human Jurkat cells (ATCC #TIB-152) with an EC₅₀ concentration of about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, about 100 nM.

In an aspect said EC₅₀ concentration is determined in a FACS assay as described herein in Example 2.2 or Example 3.5 In an aspect, said EC₅₀ concentration is determined in the Fab format. In an aspect, said EC₅₀ concentration is determined in the antibody Fv format.

In an aspect said EC₅₀ concentration is determined for a bispecific antibody format according to Example 2 or Example 3 comprising the isolated human antibody or antigen-binding fragment thereof specific for human CD3 according to the present disclosure.

In an aspect, said isolated human antibody or antigen-binding fragment thereof specific for cluster of differentiation 3 (CD3) comprises a heavy chain variable region (VH) comprising the amino acid sequence of SEQ ID NO: 15 and a light chain variable region (VL) comprising the amino acid sequence of SEQ ID NO: 21.

### ELISA Binding

In an aspect, the present disclosure provides an isolated human antibody or antigen-binding fragment thereof specific for CD3, which specifically binds to a recombinant human CD3 epsilon polypeptide comprising SEQ ID NO: 41 or SEQ ID NO: 43 with an EC₅₀ concentration between 1-40 nM, preferably between 1-15 nM. In an aspect, said isolated human antibody or antigen-binding fragment thereof specifically binds to a recombinant human CD3 epsilon polypeptide having SEQ ID NO: 41 or SEQ ID NO: 43 with an EC₅₀ of concentration of less than 40 nM, preferably less than 15 nM. In an aspect, said antibody or antigen-binding fragment thereof specifically binds to a recombinant human CD3 epsilon polypeptide having SEQ ID NO: 41 or SEQ ID NO: 43 with an EC₅₀ of concentration which is about 0.5 fold lower, about 1 fold lower, about 1.5 fold lower, about 2 fold lower, about 2.5 fold lower or about 3 fold lower as the EC₅₀ concentration determined for an antibody or antigen-binding fragment thereof specific for CD3 comprising the VH of SEQ ID NO: 14 and the VL of SEQ ID NO: 20 or the VH of SEQ ID NO: 25 and the VL of SEQ ID NO: 26.

In an aspect, said EC₅₀ concentration is determined as described herein in Example 4.7. In an aspect, said EC₅₀ concentration is determined in an ELISA assay. In an aspect, said EC₅₀ concentration is determined in for antibody Fv. In an aspect, said EC₅₀ concentration is determined for a bispecific antibody format according Example 4 comprising the isolated human antibody or antigen-binding fragment thereof specific for human CD3 according to the present disclosure.

In an aspect, said isolated human antibody or antigen-binding fragment thereof specific for cluster of differentiation 3 (CD3) comprises a heavy chain variable region (VH) comprising the amino acid sequence of SEQ ID NO: 15 and a light chain variable region (VL) comprising the amino acid sequence of SEQ ID NO: 21.

### Safety

In an aspect, the present disclosure provides an isolated human antibody or antigen-binding fragment thereof specific for CD3, wherein said antibody or antigen binding fragment thereof induces less upregulation of CD69 expression on CD4+ and/or CD8+ T cells compared to an isolated human antibody or antigen-binding fragment thereof specific for CD3 comprising the VH of SEQ ID NO: 14 and the VL of SEQ ID NO: 20.

In an aspect, the present disclosure provides an isolated human antibody or antigen-binding fragment thereof which specifically binds to CD3 expressed on CD4+ and/or CD8+ T cells and induces upregulation of CD69 expression in a lower number of CD4+ and/or CD8+ T cells compared to an antibody or antigen-binding fragment thereof specific for CD3 comprising the VH of SEQ ID NO: 14 and the VL of SEQ ID NO: 20.

In certain aspects, the upregulation of CD69 on CD4+ and/or CD8+ T cells is determined by the method as described herein in Example 4.13. In certain aspects, the upregulation of CD69 is determined for an bispecific antibody according Example 4 comprising the isolated human antibody or antigen-binding fragment thereof specific for CD3 according to the present disclosure. In an aspect, said antibody is a bispecific antibody. In an aspect, said bispecific antibody binds monovalently to CD3 expressed on CD4+ and/or CD8+ positive T cells. In an aspect, said CD69 upregulation is determined for an Fv fragment.

In certain aspects, said antibody or antigen-binding fragment thereof specific for CD3 is an antibody or antigen-binding fragment thereof listed in Table 8.

In an aspect, said isolated human antibody or antigen-binding fragment thereof specific for cluster of differentiation 3 (CD3) comprises a heavy chain variable region (VH) comprising the amino acid sequence of SEQ ID NO: 15 and a light chain variable region (VL) comprising the amino acid sequence of SEQ ID NO: 21.

### Deimmunization

T cell epitopes are specific peptide sequences within a polypeptide sequence which either bind with reasonable efficiency to MHC class II molecules or which in the form of peptide:MHC complexes bind strongly to the T cell receptors from the species to receive the (therapeutic) protein or polypeptide, or which, show the ability to stimulate T cells via presentation on MHC class II. (Potential) T cell epitopes can be measured by any computational or physical method to establish MHC binding. It is however understood that certain peptides which are found to bind to MHC class II molecules are recognized as “self” within the organism into which a protein is administered and as such do not elicit an immune response. Such peptides are found for example in germline human immunoglobulin variable region protein sequences. The present disclosure takes the approach of designing improved human antibodies specific for CD3, by removal of potential T cell epitopes present in a parent human antibody specific for CD3. This involves the choice of amino acid substitutions enabling the removal of identified T cell epitopes and testing of a range of variant molecules with different amino acid substitutions. The principle of the present disclosure is that a human CD3 specific antibody is altered in its primary CDR sequences by identification of potential T cell epitopes and their subsequent alteration within the CDRs in order to eliminate such potential T cell epitopes. The primary sequence of the therapeutic antibody can be analyzed for the presence of T cell epitopes by any suitable means. In the method of the present disclosure, the computational screening method as those provided by Lonza (Epibase™, Epibase Version: v3.0, WO 2003/105058).

In an aspect, the present disclosure provides deimmunized isolated human antibodies or antigen-binding fragment thereof specific for CD3. In an aspect, the present disclosure provides an antibody or antigen-binding fragment thereof specific for CD3, which is a deimmunized isolated human antibody or antigen-binding fragment thereof specific for CD3. In an aspect, the present disclosure provides an isolated human antibody or antigen-binding fragment thereof specific for CD3, which is less or non-immunogenic in human beings when comparted to an antibody or antigen-binding fragment thereof specific for CD3 comprising the VH of SEQ ID NO: 14 and the VL of SEQ ID NO: 20.

In an aspect, the present disclosure provides an isolated human antibody or antigen-binding fragment thereof specific for CD3, wherein said antibody or antigen-binding fragment has a reduced risk in electing an immune response or an immunogenic reaction in human beings. In an aspect, the present disclosure provides an isolated human antibody or antigen-binding fragment thereof specific for CD3, wherein said antibody or antigen-binding fragment has a reduced risk in electing an human anti-human antibody response in human beings.

In an aspect, said antibody or antigen-binding fragment thereof has a reduced risk in electing an immune response or an immunogenic reaction in human beings once administered to said human being. In an aspect, said reduced risk references to the risk in electing an immune response or an immunogenic reaction in human beings for an human antibody or antigen-binding fragment thereof comprising the VH of SEQ ID NO: 14 and a VL of SEQ ID NO: 20. In an aspect of the present disclosure, said reduced risk in electing an immune response or an immunogenic reaction in human beings is determined as described herein in Example 4. In an aspect of the present disclosure, said reduced risk is determined for the VH and/or the VL of an human antibody or antigen-binding fragment thereof specific for CD3 according to the present disclosure.

In an aspect of the present disclosure, said risk or reduced risk is provided as a numerical value determined for T cell epitopes, Risk Score, Absolut Score, H-lines, Absolut H-lines H-Score and Absolut H-Score and/or Hotspots, all of the foregoing as defined herein. In an aspect of the present disclosure, said risk or reduced risk is referenced to the risk determined for an human antibody or antigen-binding fragment thereof comprising the VH of SEQ ID NO: 14 and a VL of SEQ ID NO: 20. In an aspect, said numerical value determined for T cell epitopes, Risk Score, Absolut Score, H-lines, Absolut H-lines H-Score and Absolut H-Score and/or Hotspots using the in silico T cell epitope screening tool Epibase™, Epibase Version: v3.0 (Lonza), based on WO 2003/105058, which is incorporated herein in its entirety. In an aspect, the allele set is Major Caucasian DRB1 alleles. In an aspect, the risk is determined for different allotypes of the Caucasian DRB1 alleles. In an aspect, the filter set is for human antibody germline sequences. In an aspect, the risk is determined by excluding human antibody germline sequences present in the VH and/or the VL of an human antibody or antigen-binding fragment thereof specific for CD3 according to the present disclosure. In an aspect, the risk is determined for the HCDR1, HCDR2, HCDR3 and LCDR3 region of an human antibody or antigen-binding fragment thereof specific for CD3 according to the present disclosure. In an aspect, said risk excludes the risk for human antibody germline sequences in eliciting an immune response in human beings. In an aspect, the selected population is Caucasian. In an aspect, the isolated human antibody or antigen-binding fragment thereof specific for CD3 according to the present disclosure has a reduced risk in electing an immune response or an immunogenic reaction in human beings of a Caucasian population. In an aspect, the isolated human antibody or antigen-binding fragment thereof specific for CD3 according to the present disclosure has a reduced risk in electing an immune response or an immunogenic reaction in human beings of a Caucasian population compared to the risk of an isolated human antibody or antigen-binding fragment thereof specific for CD3 comprising the VH of SEQ ID NO: 14 and a VL of SEQ ID NO: 20.

In an aspect, the isolated human antibody or antigen-binding fragment thereof specific for CD3 according to the present disclosure has a VH with an Absolute Score as determined herein, in the range of about 380-450, preferably of about 390-425. In an aspect, the isolated human antibody or antigen-binding fragment thereof specific for CD3 according to the present disclosure has a VH with an Absolute Score of less than 440, less than 430, less than 420, less than 410, less than 400, less than 395. In an aspect, the isolated human antibody or antigen-binding fragment thereof specific for CD3 according to the present disclosure has a VH with a reduced Absolute Score compared to a VH comprising SEQ ID NO: 14. In an aspect, said reduced Absolute Score is reduced by more than 200, such as more than 210, 215, 220, 230, 235, 240, 245 or 250.

In an aspect, the isolated human antibody or antigen-binding fragment thereof specific for CD3 according to the present disclosure has a VH with an Absolute H-Score in the range of about 330-370, preferably of about 335-365. In an aspect, the isolated human antibody or antigen-binding fragment thereof specific for CD3 has a VH with an Absolute H-Score of about 335 or 365. In an aspect, the isolated human antibody or antigen-binding fragment thereof specific for CD3 according to the present disclosure has a VH with an Absolute H-Score of less than 370, less than 364, less than 360, less than 355, less than 350, less than 345, less than 340, or less than 335. In an aspect, the isolated human antibody or antigen-binding fragment thereof specific for CD3 according to the present disclosure has a VH with a reduced Absolute H-Score compared to a VH comprising SEQ ID NO: 14. In an aspect, said reduced Absolute H-Score is reduced by more than 200, such as more than 210, 215, 220, 230, 235, 240, 245 or 250.

In an aspect, the isolated human antibody or antigen-binding fragment thereof specific for CD3 according to the present disclosure has a VH with Absolute H-Lines of 14. In an aspect, the isolated human antibody or antigen-binding fragment thereof specific for CD3 according to the present disclosure has a VH with reduced Absolute H-Lines compared to a VH comprising SEQ ID NO: 14. In an aspect, said reduced Absolute H-Lines is reduced by 11. In an aspect, the isolated human antibody or antigen-binding fragment thereof specific for CD3 according to the present disclosure has a VH with Absolute Hotspots of 4. In an aspect, the isolated human antibody or antigen-binding fragment thereof specific for CD3 according to the present disclosure has a VH with reduced Absolute Hotspots compared to a VH comprising SEQ ID NO: 14. In an aspect, said reduced Absolute Hotspots is reduced by 2. In an aspect, the isolated human antibody or antigen-binding fragment thereof specific for CD3 according to the present disclosure has a VL with an Absolute Score and/or Absolute H-Score of 62. In an aspect, the isolated human antibody or antigen-binding fragment thereof specific for CD3 according to the present disclosure has a VL with a reduced Absolute Score and/or Absolute H-Score compared to a VH comprising SEQ ID NO: 14. In an aspect, the isolated human antibody or antigen-binding fragment thereof specific for CD3 according to the present disclosure has a VL with Absolute H-Lines of 2. In an aspect, the isolated human antibody or antigen-binding fragment thereof specific for CD3 according to the present disclosure has a VL with Absolute Hotspots of 0.

In an embodiment, said isolated human antibody or antigen-binding fragment thereof specific for CD3 comprises VH and a VL selected from the group consisting of:
i. the VH comprising the amino acid sequence of SEQ ID NO: 15 and the VL comprising the amino acid sequence of SEQ ID NO: 21,
ii. the VH comprising the amino acid sequence of SEQ ID NO: 16 and the VL comprising the amino acid sequence of SEQ ID NO: 21,
iii. the VH comprising the amino acid sequence of SEQ ID NO: 17 and the VL comprising the amino acid sequence of SEQ ID NO: 21,
iv. the VH comprising the amino acid sequence of SEQ ID NO: 18 and the VL comprising the amino acid sequence of SEQ ID NO: 21, and
v. the VH comprising the amino acid sequence of SEQ ID NO: 19 and the VL comprising the amino acid sequence of SEQ ID NO: 21.

### Multispecific Antibodies

The isolated human antibody or antigen-binding fragment thereof specific for CD3 according to the present disclosure is preferentially to be used in a bi- or multispecific antibody format to target CD3 expressing cytotoxic T cells and to stimulate cytotoxic T cell activation, e.g., under circumstances where T cell mediated killing of specific cell types, such as tumor cells, is beneficial or desirable.

The isolated human antibody or antigen-binding fragment thereof specific for CD3 according to the present disclosure can be linked to or co-expressed with another functional molecule, e.g. another peptide or protein. In an aspect, the present disclosure pertains to an isolated human antibody or antigen-binding fragment thereof specific for CD3, wherein said antibody or antigen-binding fragment thereof is fused to a heterologous protein or polypeptide. In an aspect, the present disclosure pertains to a fusion protein comprising an isolated human antibody or antigen-binding fragment thereof specific for CD3 according to the present disclosure and a heterologous protein or polypeptide. For example, an antibody or antigen-binding fragment thereof can be functionally linked (e.g. by chemical coupling, genetic fusion, noncovalent association or otherwise) to one or more other molecular entities, such as another antibody or antigen-binding fragment thereof to produce a bispecific or multispecific antibody with an second or optionally third binding specificity. Bispecific or multispecific antibodies, capable of binding to two or more antigens, are of great interest for therapeutic applications. In another aspect, the present disclosure provides an isolated human antibody or antigen-binding fragment thereof specific for CD3, wherein said antibody is a monospecific, bispecific or multispecific antibody or antigen-binding fragment thereof. A multispecific antibody may contain an antibody or antigen-binding fragment thereof being specific for different epitopes on the same target antigen or may contain antibody or antigen-binding fragment thereof specific for more than one target antigen. In the context of bispecific or multispecific antibodies according to the present disclosure, a cell surface target antigen may be a tumor associated antigen (TAA). Non-limiting examples of tumor associated antigen include, e.g., an antigen that is expressed on the surface of a tumor or cancerous cell. Exemplary multispecific antibody formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, Mab₂ bispecific formats (see, e.g., Klein et al. 2012, mAbs 4:6, 1-11) and Hemibodies (see e.g. Stuhler et al. Nat Commun. 2019; 10: 5387). Preferably, the isolated human antibody or antigen-binding fragment thereof specific for CD3 according to the present disclosure is used in a format as described herein in Example 3 or Example 5 or in WO 2020/115115.

In an aspect, the present disclosure provides a bispecific or multispecific antibody comprising an antigen-binding fragment of an human antibody specific for CD3 according to the present disclosure and a second antigen-binding fragment of a second antibody which binds to a different antigen than said first antigen-binding fragment thereof. In an aspect of the present disclosure, said second antigen-binding fragment binds to a cell surface antigen. In an aspect, said cell surface target antigen is a tumor associated antigen. In an aspect, said first antigen-binding fragment binds to CD3 present or expressed on an immune effector cell. In an aspect, said immune effector cell is a T cell. In an aspect, said T cell is a cytotoxic T cell. In an aspect, the present disclosure provides a bispecific or multispecific antibody comprising an human antibody or antigen-binding fragment thereof specific for CD3 according to the present disclosure, wherein the bispecific or multispecific antibody mediates redirected T cell killing of target antigen expressing cells. In certain aspects, said target cell killing can be determined by the method as described herein, such as described in Example 4.12.

In an aspects of the present disclosure, the bispecific or multispecific antibody according to the present disclosure specifically binds to CD3 expressed on a T cell and to a second antigen present on a cell other than the T cell. In certain aspects, said bispecific or multispecific antibody activates T cells following binding to CD3 expressed on a T cell and binding to a second antigen present on a target cell other than the T cell. In certain aspects, the activated T cell is capable of exerting a cytotoxic effect and/or an apoptotic effect on the other cell. In an aspect, the present disclosure provides a bispecific or multispecific antibody comprising the human antibody or antigen-binding fragment thereof specific for CD3 according to the present disclosure, wherein said bispecific or multispecific antibody induces human T cell proliferation in the presence of a cell surface target antigen expressing cell following binding to CD3 expressed on a T cell and to the cell surface target expressing cell.

### Linker Optimization

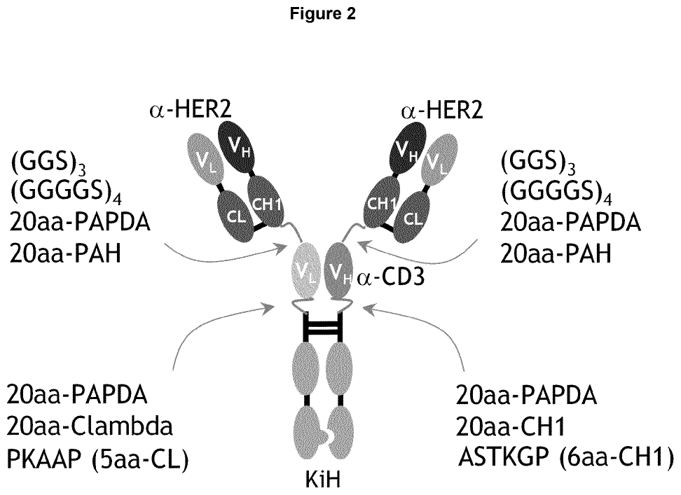

In an aspect, the bispecific antibody according to the present disclosure is composed of three Fv regions. This is achieved by using a regular immunoglobulin (e.g. IgG) antibody structure (two heavy chains with associated two light chains that form two Fv regions) that incorporates an additional Fv region between the two Fab arms and the Fc portion of the regular immunoglobulin structure. In an aspect, the bispecific antibody according to the present disclosure has a general structure as depicted in FIG. 2.

In an aspect, the present disclosure provides a bispecific antibody, comprising
a) a first Fab comprising a first Fv region, which specifically binds to a first antigen,
b) a second Fv region which specifically binds to a second antigen and
c) a second Fab comprising a third Fv region, which specifically binds to a third antigen, and
d) a Fc region composed of a first and second Fc region subunit, wherein
   i. the C-terminus of the first Fab heavy chain is fused to the N-terminus of the VH or VL of the second Fv region via a first peptide linker, wherein
   ii. the C-terminus of the VH or VL of the second Fv region is fused to the N-terminus of the first Fc region subunit via a second peptide linker, wherein
   iii. the N-terminus of the second Fc region subunit is fused to the C-terminus of the complementary variable domain of the second Fv region via a fourth peptide linker, and wherein
   iv. the C-terminus of the second Fab heavy chain is fused to the N-terminus of the VH or VL of the second Fv region via a third peptide linker with the proviso that the first and second Fab are fused to distinct variable regions of the second Fv region.

In an aspect, the C-terminus of the CH1 domain of the first Fab is fused to the N-terminus of the VH or VL of the second Fv region via the first peptide linker with the proviso that the first and second Fab are fused to distinct variable domains of the second Fv region. In an aspect, the C-terminus of the CH1 domain of the first Fab is fused to the N-terminus of the VH of the second Fv region via the first peptide linker with the proviso that the first and second Fab are fused to distinct variable domains of the second Fv region. In an aspect, the C-terminus of the CH1 domain of the first Fab is fused to the N-terminus of the VL of the second Fv region via the first peptide linker with the proviso that the first and second Fab are fused to distinct variable domains of the second Fv region.

In an aspect, the C-terminus of the CH1 domain of the second Fab is fused to the N-terminus of the VH or VL of the second Fv region via the third peptide linker with the proviso that the first and second Fab are fused to distinct variable domains of the second Fv region. In an aspect, the C-terminus of the CH1 domain of the second Fab is fused to the N-terminus of the VH of the second Fv region via the third peptide linker with the proviso that the first and second Fab are fused to distinct variable domains of the second Fv region. In an aspect, the C-terminus of the CH1 domain of the second Fab is fused to the N-terminus of the VL of the second Fv region via the third peptide linker with the proviso that the first and second Fab are fused to distinct variable domains of the second Fv region.

In an aspect, the C-terminus of the heavy chain of the first Fab is fused to the N-terminus of the VL of the second Fv region via the first peptide linker and the C-terminus of the heavy chain of the second Fab is fused to the N-terminus of the VH of the second Fv region via the third peptide linker. In an aspect, the C-terminus of the heavy chain of the first Fab is fused to the N-terminus of the VH of the second Fv region via the first peptide linker and the C-terminus of the heavy chain of the second Fab is fused to the N-terminus of the VL of the second Fv region via the third peptide linker.

In an aspect, the bispecific antibody according to the present disclosure comprises 4 polypeptides, wherein
a) the first polypeptide comprises the light chain of the first Fab,
b) the second polypeptide comprises from its N-terminus to its C-terminus
   i. the heavy chain of the first Fab,
   ii. the first peptide linker,
   iii. the VL of the second Fv region,
   iv. the second peptide linker, and
   v. the first Fc region subunit
c) the third polypeptide comprises from its N-terminus to its C-terminus
   i. the heavy chain of the second Fab,
   ii. the third peptide linker,
   iii. the VH of the second Fv region,
   iv. the fourth peptide linker
   v. the second Fc region subunit, and
d) the fourth polypeptide comprises the light chain of the second Fab.

In an aspect, the bispecific antibody according to the present disclosure comprises 4 polypeptides, wherein
a) the first polypeptide comprises the light chain of the first Fab,
b) the second polypeptide comprises from its N-terminus to its C-terminus
   i. the heavy chain of the first Fab,
   ii. the first peptide linker,
   iii. the VH of the second Fv region,
   iv. the second peptide linker, and
   iii. the first Fc region subunit
c) the third polypeptide comprises from its N-terminus to its C-terminus
   i. the heavy chain of the second Fab,
   ii. the third peptide linker,
   iii. the VL of the second Fv region,
   iv. the fourth peptide linker, and
   v. the second Fc region subunit
d) the fourth polypeptide comprises the light chain of the second Fab.

In an aspect of the present disclosure, the first, second, third and fourth peptide linker is selected from the group of amino acid sequences consisting of:

a)
(SEQ ID NO: 30)
GGSGGSGGS,
b)
(SEQ ID NO: 31)
GGGGGGGGSGGGGSGGGGS,
c)
(SEQ ID NO: 33)
AQPAAPAPDAHEAPAPAQGS,
d)
(SEQ ID NO: 32)
AHPAAPAPAHPAAPAPAHGH,
e)
(SEQ ID NO: 36)
PKAAP,
f)
(SEQ ID NO: 34)
PKAAPSVTLFPPSSEELQAN,
g)
(SEQ ID NO: 37)
ASTKGP,
and
h)
(SEQ ID NO: 35)
ASTKGPSVFPLAPSSKSTSG

In an aspect of the present disclosure, the second and fourth peptide linker is C-terminally fused to the amino acid sequence of DKTHTCPPCP (SEQ ID NO: 38). In an aspect of the present disclosure, the second and fourth peptide linker additionally comprises at the C-terminus the amino acid sequence of DKTHTCPPCP (SEQ ID NO: 38).

In an aspect of the present disclosure, the second and fourth peptide linker is selected from the group of amino acid sequence consisting of:

(SEQ ID NO: 76)
a) PKAAPDKTHTCPPCP,
(SEQ ID NO: 77)
b) ASTKGPDKTHTCPPCP,
and
(SEQ ID NO: 78)
c) AQPAAPAPDAHEAPAPAQGSDKTHTCPPCP,
(SEQ ID NO: 79)
d) PKAAPSVTLFPPSSEELQANDKTHTCPPCP,
(SEQ ID NO: 80)
e) ASTKGPSVFPLAPSSKSTSGDKTHTCPPCP

In an aspect of the present disclosure,
a) the first and third peptide linker comprises the amino acid sequence GGGGSGGGGSGGGGSGGGGS (SEQ ID NO: 31), the second peptide linker comprises the amino acid sequence of PKAAP (SEQ ID NO: 36), and the fourth peptide linker comprises the amino acid sequence of ASTKGP (SEQ ID NO: 37), or
b) the first and third peptide linker comprises the amino acid sequence GGGGSGGGGSGGGGSGGGGS (SEQ ID NO: 31) and the second and fourth peptide linker comprises the amino acid sequence of AQPAAPAPDAHEAPAPAQGS (SEQ ID NO: 33), or
c) the first and third peptide linker comprises the amino acid sequence GGGGSGGGGSGGGGSGGGGS (SEQ ID NO: 31), the second peptide linker comprises the amino acid sequence of PKAAPSVTLFPPSSEELQAN (SEQ ID NO: 34), and the fourth peptide linker comprises the amino acid sequence of ASTKGPSVFPLAPSSKSTSG (SEQ ID NO: 35), or
d) the first and third peptide linker comprises the amino acid sequence of AHPAAPAPAHPAAPAPAHGH (SEQ ID NO: 32), the second peptide linker comprises the amino acid sequence of PKAAPSVTLFPPSSEELQAN (SEQ ID NO: 34), and the fourth peptide linker comprises the amino acid sequence of ASTKGPSVFPLAPSSKSTSG (SEQ ID NO: 35), or
e) the first and third peptide linker comprises the amino acid sequence of AQPAAPAPDAHEAPAPAQGS (SEQ ID NO: 33), the second peptide linker comprises the amino acid sequence of PKAAPSVTLFPPSSEELQAN (SEQ ID NO: 34), and the fourth peptide linker comprises the amino acid sequence of ASTKGPSVFPLAPSSKSTSG (SEQ ID NO: 35), or
f) the first and third peptide linker comprises the amino acid sequence of GGSGGSGGS (SEQ ID NO: 30), the second peptide linker comprises the amino acid sequence of PKAAPSVTLFPPSSEELQAN (SEQ ID NO: 34), and the fourth peptide linker comprises the amino acid sequence of ASTKGPSVFPLAPSSKSTSG (SEQ ID NO: 35).

In an aspect of the present disclosure,
a) the first and third peptide linker comprises the amino acid sequence GGGGSGGGGSGGGGSGGGGS (SEQ ID NO: 31), the second peptide linker comprises the amino acid sequence of PKAAPDKTHTCPPCP (SEQ ID NO: 76), and the fourth peptide linker comprises the amino acid sequence of ASTKGPDKTHTCPPCP (SEQ ID NO: 77), or
b) the first and third peptide linker comprises the amino acid sequence GGGGSGGGGSGGGGSGGGGS (SEQ ID NO: 31) and the second and fourth peptide linker comprises the amino acid sequence of AQPAAPAPDAHEAPAPAQGSDKTHTCPPCP (SEQ ID NO: 78), or
c) the first and third peptide linker comprises the amino acid sequence GGGGSGGGGSGGGGSGGGGS (SEQ ID NO: 31), the second peptide linker comprises the amino acid sequence of PKAAPSVTLFPPSSEELQANDKTHTCPPCP (SEQ ID NO: 79), and the fourth peptide linker comprises the amino acid sequence of ASTKGPSVFPLAPSSKSTSGDKTHTCPPCP (SEQ ID NO: 80), or
d) the first and third peptide linker comprises the amino acid sequence of AHPAAPAPAHPAAPAPAHGH (SEQ ID NO: 32), the second peptide linker comprises the amino acid sequence of PKAAPSVTLFPPSSEELQANDKTHTCPPCP (SEQ ID NO: 79), and the fourth peptide linker comprises the amino acid sequence of ASTKGPSVFPLAPSSKSTSGDKTHTCPPCP (SEQ ID NO: 80), or
e) the first and third peptide linker comprises the amino acid sequence of AQPAAPAPDAHEAPAPAQGS (SEQ ID NO: 33), the second peptide linker comprises the amino acid sequence of PKAAPSVTLFPPSSEELQANDKTHTCPPCP (SEQ ID NO: 79), and the fourth peptide linker comprises the amino acid sequence of ASTKGPSVFPLAPSSKSTSGDKTHTCPPCP (SEQ ID NO: 80), or
f) the first and third peptide linker comprises the amino acid sequence of GGSGGSGGS (SEQ ID NO: 30), the second peptide linker comprises the amino acid sequence of PKAAPSVTLFPPSSEELQANDKTHTCPPCP (SEQ ID NO: 79), and the fourth peptide linker comprises the amino acid sequence of ASTKGPSVFPLAPSSKSTSGDKTHTCPPCP (SEQ ID NO: 80).

In an aspect of the present disclosure, the first and third peptide linker comprises the amino acid sequence GGGGSGGGGSGGGGSGGGGS (SEQ ID NO: 31) and the second and fourth peptide linker comprises the amino acid sequence of AQPAAPAPDAHEAPAPAQGS (SEQ ID NO: 33). In an aspect of the present disclosure, the first and third peptide linker comprises the amino acid sequence GGGGSGGGGSGGGGSGGGGS (SEQ ID NO: 31) and the second and fourth peptide linker comprises the amino acid sequence of AQPAAPAPDAHEAPAPAQGSDKTHTCPPCP (SEQ ID NO: 78).

In an aspect of the present disclosure, the second Fv region specifically binds to CD3. In an aspect, the second Fv is specific for CD3, in particular for human CD3, more particular for human CD3 epsilon.

In an aspect of the present disclosure, the second Fv comprises
i. a heavy chain variable region (VH) comprising
   (a) a heavy chain complementary determining region (HCDR)1 comprising the amino acid sequence of GFSFGSHYMS (SEQ ID NO: 1),
   (b) a HCDR2 comprising the amino acid sequence of NINQIGYSSYYVESVKG (SEQ ID NO: 2), NINQIGYSSYYGESVKG (SEQ ID NO: 3) or NINQIGYSSYYEESVKG (SEQ ID NO: 4), and
   (c) a HCDR3 comprising the amino acid sequence of GYSAEFAHRSGLDV (SEQ ID NO: 5), GYSDEFATRSGLDV (SEQ ID NO: 6), GYSEEFAHRSGLDV (SEQ ID NO: 7), GYSDEFAKRSGLDV (SEQ ID NO: 8) or GYSDEFAHRSGLDV (SEQ ID NO: 9), and
ii. a variable light chain region (VL) comprising
   (d) a light chain complementary determining region (LCDR)1 comprising the amino acid sequence of SGSSSNIGSNYVY (SEQ ID NO: 10),
   (e) a LCDR2 comprising the amino acid sequence of RNNQRPS (SEQ ID NO: 11), and
   (f) a LCDR3 comprising the amino acid sequence of AGWSRSLHGAV (SEQ ID NO: 12) or AGWSRELHGAV (SEQ ID NO: 13).

In an aspect of the present disclosure, the second Fv comprises a VH and VL selected from the group consisting of:
i. the VH comprising the amino acid sequence of SEQ ID NO: 14 and the VL comprising the amino acid sequence of SEQ ID NO: 20,
ii. the VH comprising the amino acid sequence of SEQ ID NO: 15 and the VL comprising the amino acid sequence of SEQ ID NO: 21,
iii. the VH comprising the amino acid sequence of SEQ ID NO: 16 and the VL comprising the amino acid sequence of SEQ ID NO: 21,
iv. the VH comprising the amino acid sequence of SEQ ID NO: 17 and the VL comprising the amino acid sequence of SEQ ID NO: 21,
v. the VH comprising the amino acid sequence of SEQ ID NO: 18 and the VL comprising the amino acid sequence of SEQ ID NO: 21, and
vi. the VH comprising the amino acid sequence of SEQ ID NO: 19 and the VL comprising the amino acid sequence of SEQ ID NO: 21.

In an aspect of the present disclosure, the light chain of the first Fab or second Fab comprises the VL and CL of the first Fab or second Fab, respectively. In an aspect of the present disclosure, the light chain of the first Fab and the second Fab are identical. In an aspect of the present disclosure, the heavy chain of the first Fab and the second Fab are identical. In an aspect of the present disclosure, the first Fab and the second Fab are identical.

In an aspect, the first and second Fc region subunit forms a Fc region. In an aspect, the Fc region is an IgG1 Fc region. In an aspect, said IgG1 Fc region is a human IgG1 Fc region. In an aspect, the Fc region comprises one or more amino acid modifications promoting the association of the first and second Fc region subunit. In an aspect, in the CH3 domain of first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index. In an aspect of the present disclosure, the Fc region of the bispecific antibody according to the present disclosure has a reduced binding affinity to an Fc receptor and/or to C1q and/or has reduced effector function. In an aspect of the present disclosure, Fc region comprises one or more amino acid mutations in each Fc region subunit, wherein said one or more amino acid mutations are selected from the group consisting of: L234A, L235E, G237A, A330S and P331S with numbering according EU index. In an aspect, in each Fc region subunit, at least 5 amino acid residues in the positions corresponding to positions L234, L235, G237, A330, P331 with numbering according EU index in a human IgG1 are mutated to A, E, A, S, and S, respectively

In an aspect, the first antigen and the third antigen are identical. In an aspect, the first and third antigen is a tumor-associated antigen. In an aspect, the first and third antigen is a tumor-associated antigen expressed on a tumor cell or cancerous cell. In an aspect, the second antigen is an immune cell related antigen. In an aspect, the second antigen is expressed on an immune cell. In an aspect, the second antigen is expressed on an immune effector cell. In an aspect, the second antigen is expressed on a cytotoxic T cell. In an aspect, the second antigen is CD3. In an aspect, the second antigen is CD3 epsilon. In an aspect, the second antigen is human CD3. In an aspect, the second antigen is human CD3 epsilon. In an aspect, the bispecific antibody according to the present disclosure provides bivalent binding to the first antigen and monovalent binding to the second antigen. In an aspect, said bispecific antibody is a trivalent bispecific antibody.

### Efficacy

In certain aspects, the present disclosure provides a bispecific antibody according to the present disclosure comprising an human antibody or antigen-binding fragment thereof specific for CD3 according to the present disclosure, wherein said bispecific antibody mediates target cell killing of target antigen expressing cells. In an aspect, that target cell killing is mediated in the present of cytotoxic T cells. In certain aspects, said bispecific antibody comprises as second antigen-binding fragment of an antibody, which specifically binds to a cell surface target antigen. In certain aspects, said target antigen is a tumor associated antigen. In an aspect, that target antigen is HER2. In an aspect, that second antigen binding fragment of an antibody binds to HER2 (UniProtKB—P04626). In an aspect, that target cell is a tumor cell or a cancer cell. In an aspect that target cell is a SKOV-3 (ATCC® HTB-77™), SKBR3 (ATCC® HTB-30™) or MCF-7 (ATCC® HTB-22™) cell. In certain aspects, target cell killing is determined by the method as described herein in Example 4.13.

### Treatment

In an aspect, the present disclosure provides an isolated human antibody or antigen-binding fragment thereof specific for CD3 or a bispecific antibody according to the present disclosure for use as a medicament. In an aspect, the present disclosure refers to an isolated human antibody or antigen-binding fragment thereof specific for CD3 or a bispecific antibody according to the present disclosure for use in the preparation or manufacture of a medicament. In an aspect, the present disclosure provides an isolated human antibody or antigen-binding fragment thereof specific for CD3 or a bispecific antibody according to the present disclosure for use in enhancing immune function in a subject having a cell proliferative disease.

In an aspect, the present disclosure provides a method of treating or delaying the progression of a cell proliferative disease in a subject in need thereof, the method comprising administering to said subject an effective amount of an isolated human antibody or antigen-binding fragment thereof specific for CD3 or a bispecific antibody according the present disclosure. In an aspect, said isolated human antibody or antigen-binding fragment thereof specific for CD3 or the bispecific antibody according the present disclosure is for use in treating or delaying progression of a cell proliferative disease in a subject in need thereof. In an aspect, said cell proliferative disease is a cancer. In an aspect, the said cancer is selected from the group consisting but not limited to: esophageal cancer, stomach cancer, small intestine cancer, large intestine cancer, colorectal cancer, breast cancer, non-small cell lung cancer, non-Hodgkin's lymphoma (NHL), B cell lymphoma, B cell leukemia, multiple myeloma, renal cancer, prostate cancer, liver cancer, head and neck cancer, melanoma, ovarian cancer, mesothelioma, glioblastoma, germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

In an aspect, the present disclosure provides the use of an isolated human antibody or antigen-binding fragment thereof specific for CD3 or bispecific antibody according to the present disclosure for the manufacture of a medicament for treating or delaying progression of a cell proliferative disease. In an aspect, the present disclosure provides the use of an isolated human antibody or antigen-binding fragment thereof specific for CD3 or a bispecific antibody according to the present disclosure for the manufacture of a medicament for enhancing immune function in a subject having a cell proliferative disease. In an aspect, the present disclosure provides a method of treating a subject in need thereof with an isolated human antibody or antigen-binding fragment thereof specific for CD3 or an bispecific antibody according to the present disclosure.

In an aspect, the isolated human antibody or antigen-binding fragment thereof specific for CD3 or the bispecific antibody according to the present disclosure or a pharmaceutical composition comprising said antibody or antigen-binding fragment thereof or bispecific antibody is administered subcutaneously, intravenously, intramuscularly, topically, orally, transdermally, intraperitoneally, intraorbitally, by implantation, by inhalation, intrathecally, intraventricularly, or intranasally.

### Compositions

In an aspect, the present disclosure provides a pharmaceutical composition comprising an isolated human antibody or antigen-binding fragment thereof specific for CD3 or bispecific antibody according to the present disclosure and a pharmaceutically acceptable carrier or excipient. Such carriers or excipients are well known in the art, and the skilled artisan will find a formulation and a route of administration best suited to treat a subject with the antibodies or antigen-binding fragment thereof according to the present disclosure. In an aspect, the present disclosure pertains to the use of a pharmaceutical compositions comprising an isolated human antibody or antigen-binding fragment thereof specific for CD3 or bispecific antibody according to the present disclosure in the preparation of a medicament for the treatment of a disease. In an aspect, the present disclosure pertains to the use of said pharmaceutical composition for the treatment of a disease. In an aspect, the present disclosure provides a method of treating a cell proliferative disease in a subject, said method comprising administering to said subject a pharmaceutical composition comprising a therapeutically effective amount of an isolated human antibody or antigen-binding fragment thereof specific for CD3 or bispecific antibody according to the present disclosure. In an aspect, the present disclosure provides a pharmaceutical composition comprising a combination of an isolated human antibody or antigen-binding fragment thereof specific for CD3 or bispecific antibody according to the present disclosure and a second therapeutic agent. In an aspect, said second therapeutic agent is any agent that is advantageously combined with said human antibody or antigen-binding fragment thereof or the bispecific antibody according to the present disclosure. The present disclosure provides a therapeutic method for stimulating T cell activation using the isolated human antibody or antigen-binding fragment thereof or specific for CD3 the bispecific antibody according to the present disclosure, wherein the therapeutic method comprise administering a therapeutically effective amount of a pharmaceutical composition comprising an isolated human antibody or antigen-binding fragment thereof specific for CD3 or the bispecific antibody according to the present disclosure to a subject in need thereof. The present disclosure also provides a therapeutic method for redirecting T cell killing to a cancerous cell or tissue using an isolated human antibody or antigen-binding fragment thereof specific for CD3 or bispecific antibody according to the present disclosure, wherein the therapeutic method comprises administering a therapeutically effective amount of a pharmaceutical composition comprising said antibody or antigen-binding fragment thereof or bispecific antibody according to the present disclosure to a subject in need thereof.

### Production

In another aspect, the present disclosure refers to a method of producing an isolated human antibody or antigen-binding fragment thereof specific for CD3 of any of the antibodies listed in Tables 5-7.

The coding sequences for the heavy and light chains of an antibody or antigen-binding fragment thereof specific for CD3 according to the present disclosure can be recombinant DNA molecules, which are introduced into expression vectors by operatively linking the DNA to the necessary expression control regions (e.g. regulatory regions) required for gene expression. The skilled person will realize that the polynucleotides encoding the heavy or light chain can be cloned into different vectors or in the same vector. The vectors can be introduced into the appropriate host cells such as prokaryotic (e.g., bacterial) or eukaryotic (e.g., yeast or mammalian) cells by methods well known in the art (see e.g., “Current Protocol in Molecular Biology”, Ausubel et al. (eds.), Greene Publishing Assoc. and John Wiley Interscience, New York, 1989 and 1992). Numerous cloning vectors are known to those of skill in the art, and the selection of an appropriate cloning vector is a matter of choice. The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. Upon expression in host cells, the antibody or antigen-binding fragment thereof of the present disclosure is obtained. These steps can be achieved in different ways, as will be known by the person skilled in the art. In general, such steps typically include transforming or transfecting a suitable host cell with a nucleic acid or vector or an infectious particle which encodes the antibody molecule. Further, such steps typically include culturing said host cells under conditions suitable for the proliferation (multiplication, growth) of said host cells and a culturing step under conditions suitable for the production (expression, synthesis) of the encoded antibody or antigen-binding fragments. The culturing of host cells under conditions suitable for proliferation or expression thereof is typically accomplished in the presence of media comprising components suitable for cell growth or induction of expression. In particular aspects, the methods for the production of antibody or antigen-binding fragment thereof of the present disclosure further comprise the step of isolating the produced antibody or antigen-binding fragment thereof from the host cells or medium. Depending on the expression system and host selected, the antibody or antigen-binding fragment thereof of the present disclosure are produced by growing host cells transformed by an expression vector described above under conditions whereby the protein of interest is expressed. The protein is then isolated from the host cells and purified. If the expression system secretes the protein into growth media, the protein can be purified directly from the media. If the protein is not secreted, it is isolated from cell lysates or recovered from the cell membrane fraction. The selection of the appropriate growth conditions and recovery methods are within the skill of the art. The antibody or antigen-binding fragment thereof of the present disclosure can then be purified by a number of techniques as known to the person skilled in the art. It should be noted that Fabs of the disclosure are not naturally occurring proteins. The present disclosure also provides recombinant expression vectors capable of expressing a polypeptide comprising a heavy or light chain variable region of an antibody or antigen-binding fragment thereof specific for CD3 according to the present disclosure. For example, the present disclosure includes recombinant expression vectors encoding any of the amino acid sequences mentioned in Tables 5-7. Also included within the scope of the present disclosure are host cells into which such vectors have been introduced, as well as methods of producing the antibodies or portions thereof by culturing the host cells under conditions permitting production of the antibodies or antigen-binding fragment thereof, and recovering the antibodies and antigen-binding fragments thereof so produced.

### Antigen Sequences

TABLE 2
Amino acid sequences of recombinant human and cynomolgus CD3 epsilon
extracellular domain antigens fused to human IgG Fc or FLAG-tag as used in the
present examples.

| | SEQ | |
|---|---|---|
| | ID | |
| CD3 antigen | NO: | [aa] |
| Mature human CD3e(22-49)_ | 41 | QDGNEEMGGITQTPYKVSISGTTVILTCDIKTHTCPPCPA |
| Fc2(K105-K330) | | PELLGGPSVFLFPPKPKDTLMISRTPEVTCVVVDVSHEDP |
| | | EVKFNWYVDGVEVHNAKTKPREEQYNSTYRVVSVLTVL |
| | | HQDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPGK |
| Mature cynomolgus CD3e(22- | 42 | QDGNEEMGSITQTPYQVSISGTTVILTCDIKTHTCPPCPA |
| 49)_ | | PELLGGPSVFLFPPKPKDTLMISRTPEVTCVVVDVSHEDP |
| Fc2(K105-K330) | | EVKFNWYVDGVEVHNAKTKPREEQYNSTYRVVSVLTVL |
| | | HQDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPGK |
| Mature human CD3e(22- | 43 | QDGNEEMGGITQTPYKVSISGTTVILTCPQYPGSEILWQH |
| 118)_FLAG-chLys_Avi | | NDKNIGGDEDDKNIGSDEDHLSLKEFSELEQSGYYVCYP |
| | | RGSKPEDANFYLYLRARVDIDYKDDDDKIEGRMDKVFGR |
| | | CELAAAMKRHGLDNYRGYSLGNWVCAAKFESNFNTQAT |
| | | NRNTDGSTDYGILQINSRWWCNDGRTPGSRNLCNIPCSA |
| | | LLSSDITASVNCAKKIVSDGNGMNAWVAWRNRCKGTDV |
| | | QAWIRGCRLVNSRGLNDIFEAQKIEWHE |
| Mature cynomolgus CD3e(22- | 44 | QDGNEEMGSITQTPYQVSISGTTVILTCSQHLGSEAQWQ |
| 109)_FLAG-chLys_Avi | | HNGKNKEDSGDRLFLPEFSEMEQSGYYVCYPRGSNPED |
| | | ASHHLYLKARVVIDYKDDDDKIEGRMDKVFGRCELAAAM |
| | | KRHGLDNYRGYSLGNWVCAAKFESNFNTQATNRNTDGS |
| | | TDYGILQINSRWWCNDGRTPGSRNLCNIPCSALLSSDITA |
| | | SVNCAKKIVSDGNGMNAWVAWRNRCKGTDVQAWIRGC |
| | | RLVNSRGLNDIFEAQKIEWHE |

### Antibody Sequences

TABLE 3
Antibody variable light chain (VL) and variable heavy chain (VH) region sequences of
the anti-HER2 antibody trastuzumab as used in the examples of the present
disclosure.

| | | SEQ ID | |
|---|---|---|---|
| Antibody | Chain | NO: | [aa] |
| Trastuzumab | VH | 28 | QVQLVESGGGLVQPGGSLRLSCAASGFNIKDTYIHWVR |
| | | | QAPGKGLEWVARIYPTNGYTRYADSVKGRFTISADTSKN |
| | | | TAYLQMNSLRAEDTAVYYCSRWGGDGFYAMDYWGQG |
| | | | TLVTVSS |
| | VL | 29 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRT |

TABLE 4
Antibody variable light chain (VL) and variable heavy chain (VH) region and CDR
sequences of grandparent CD3 specific antibody CD3-MAB_{GP} as described in
PCT/EP2021/076052 (CDR sequences are provided in combined Kabat + Chothia
annotation).

| | | SEQ | |
|---|---|---|---|
| | VH/VL/ | ID | |
| Antibody | CDR | NO: | [aa] |
| CD3- | VH | 25 | EVQLVESGGGLVQPGGSLRLSCAASGFTFSSYWMSWVRQAPGKG |
| MAB_{GP} | | | LEWVANIKQDGSEKYYVDSVKGRFTISRDNAKNSLYLQMNSLRAED |
| | | | TAVYYCARGYSAEFAHRSGLDVWGQGTLVTVSS |
| | VL | 26 | QSVLTQPPSASGTPGQRVTISCSGSSSNIGSNYVYWYQQLPGTAPK |
| | | | LLIYRNNQRPSGVPDRFSGSKSGTSASLAISGLRSEDEADYYCAAW |
| | | | DHHRSGAVFGGGTKLTVLGQ |
| | HCDR1 | 22 | GFTFSSYWMS |
| | HCDR2 | 23 | NIKQDGSEKYYVDSVKG |
| | HCDR3 | 5 | GYSAEFAHRSGLDV |
| | LCDR1 | 10 | SGSSSNIGSNYVY |
| | LCDR2 | 11 | RNNQRPS |
| | LCDR3 | 24 | AAWDHHRSGAV |

TABLE 5
Antibody VL, VH and CDR sequences of the affinity optimized antibodies CD3-
MABopt_VL and CD3-MABopt_VH#1, both derivatives of grandparent antibody CD3-
MAB_{GP} (CDRs are provided in combined Kabat + Chothia annotation).

| | | SEQ | |
|---|---|---|---|
| | VH/VL/ | ID | |
| Antibody | CDR | NO: | [aa] |
| CD3- | VH | 25 | EVQLVESGGGLVQPGGSLRLSCAASGFTFSSYWMSWVRQAPG |
| MABₒₚₜ_VL | | | KGLEWVANIKQDGSEKYYVDSVKGRFTISRDNAKNSLYLQMNSL |
| (LCDR3 | | | RAEDTAVYYCARGYSAEFAHRSGLDVWGQGTLVTVSS |
| derivative of | VL | 20 | QSVLTQPPSASGTPGQRVTISCSGSSSNIGSNYVYWYQQLPGTA |
| CD3-MAB_{GP}) | | | PKLLIYRNNQRPSGVPDRFSGSKSGTSASLAISGLRSEDEADYY |
| | | | CAGWSRSLHGAVFGGGTKLTVLGQ |
| | HCDR1 | 22 | GFTFSSYWMS |
| | HCDR2 | 23 | NIKQDGSEKYYVDSVKG |
| | HCDR3 | 5 | GYSAEFAHRSGLDV |
| | LCDR1 | 10 | SGSSSNIGSNYVY |
| | LCDR2 | 11 | RNNQRPS |
| | LCDR3 | 12 | AGWSRSLHGAV |
| CD3- | VH | 14 | EVQLVESGGGLVQPGGSLRLSCAASGFSFGSHYMSWVRQAPG |
| MABₒₚₜ_VH#1 | | | KGLEWVANINQIGYSSYYVESVKGRFTISRDNAKNSLYLQMNSL |
| (HCDR1-2 | | | RAEDTAVYYCARGYSAEFAHRSGLDVWGQGTLVTVSS |
| derivative of | VL | 26 | QSVLTQPPSASGTPGQRVTISCSGSSSNIGSNYVYWYQQLPGTA |
| CD3-MAB_{GP}) | | | PKLLIYRNNQRPSGVPDRFSGSKSGTSASLAISGLRSEDEADYY |
| | | | CAAWDHHRSGAVFGGGTKLTVLGQ |
| | HCDR1 | 1 | GFSFGSHYMS |
| | HCDR2 | 2 | NINQIGYSSYYVESVKG |
| | HCDR3 | 5 | GYSAEFAHRSGLDV |
| | LCDR1 | 10 | SGSSSNIGSNYVY |
| | LCDR2 | 11 | RNNQRPS |
| | LCDR3 | 24 | AAWDHHRSGAV |

TABLE 6
Antibody VL, VH and CDR sequences of cross-clone CD3-MABopt_cc comprising the
variable heavy chain region (VH) of affinity optimized antibody CD3-MABₒₚₜ_VH#1 and the
variable light chain region (VL) of affinity optimized antibody CD3-MABₒₚₜ_VL (CDR
sequences are provided in combined Kabat + Chothia annotation)

| | | SEQ | |
|---|---|---|---|
| | VH/VL/ | ID | |
| Antibody | CDR | NO: | [aa] |
| CD3- | VH | 14 | EVQLVESGGGLVQPGGSLRLSCAASGFSFGSHYMSWVRQAPGKG |
| MABₒₚₜ_cc | | | LEWVANINQIGYSSYYVESVKGRFTISRDNAKNSLYLQMNSLRAEDT |
| | | | AVYYCARGYSAEFAHRSGLDVWGQGTLVTVSS |
| | VL | 20 | QSVLTQPPSASGTPGQRVTISCSGSSSNIGSNYVYWYQQLPGTAPK |
| | | | LLIYRNNQRPSGVPDRFSGSKSGTSASLAISGLRSEDEADYYCAGW |
| | | | SRSLHGAVFGGGTKLTVLGQ |
| | HCDR1 | 1 | GFSFGSHYMS |
| | HCDR2 | 2 | NINQIGYSSYYVESVKG |
| | HCDR3 | 5 | GYSAEFAHRSGLDV |
| | LCDR1 | 10 | SGSSSNIGSNYVY |
| | LCDR2 | 11 | RNNQRPS |
| | LCDR3 | 12 | AGWSRSLHGAV |

TABLE 7
Antibody VL, VH and CDR sequences of deimmunized CDR variants of cross-clone
CD3-MABopt_cc (CDR sequences are provided in combined Kabat + Chothia
annotation)

| | | SEQ | |
|---|---|---|---|
| | VH/VL/ | ID | |
| Antibody | CDR | NO: | [aa] |
| CD3- | VH | 18 | EVQLVESGGGLVQPGGSLRLSCAASGFSFGSHYMSWVRQAPG |
| MAB_{deimm}_1 | | | KGLEWVANINQIGYSSYYEESVKGRFTISRDNAKNSLYLQMNSL |
| | | | RAEDTAVYYCARGYSEEFAHRSGLDVWGQGTLVTVSS |
| | VL | 21 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYY |
| | | | CAGWSRELHGAVFGGGTKLTVLGQ |
| | HCDR1 | 1 | GFSFGSHYMS |
| | HCDR2 | 4 | NINQIGYSSYYEESVKG |
| | HCDR3 | 7 | GYSEEFAHRSGLDV |
| | LCDR1 | 10 | SGSSSNIGSNYVY |
| | LCDR2 | 11 | RNNQRPS |
| | LCDR3 | 13 | AGWSRELHGAV |
| CD3- | VH | 19 | EVQLVESGGGLVQPGGSLRLSCAASGFSFGSHYMSWVRQAPG |
| MAB_{deimm}_2 | | | KGLEWVANINQIGYSSYYGESVKGRFTISRDNAKNSLYLQMNSL |
| | | | RAEDTAVYYCARGYSDEFAHRSGLDVWGQGTLVTVSS |
| | VL | 21 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYY |
| | | | CAGWSRELHGAVFGGGTKLTVLGQ |
| | HCDR1 | 1 | GFSFGSHYMS |
| | HCDR2 | 3 | NINQIGYSSYYGESVKG |
| | HCDR3 | 9 | GYSDEFAHRSGLDV |
| | LCDR1 | 10 | SGSSSNIGSNYVY |
| | LCDR2 | 11 | RNNQRPS |
| | LCDR3 | 13 | AGWSRELHGAV |
| CD3- | VH | 15 | EVQLVESGGGLVQPGGSLRLSCAASGFSFGSHYMSWVRQAPG |
| MAB_{deimm}_3 | | | KGLEWVANINQIGYSSYYGESVKGRFTISRDNAKNSLYLQMNSL |
| | | | RAEDTAVYYCARGYSDEFATRSGLDVWGQGTLVTVSS |
| | VL | 21 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYY |
| | | | CAGWSRELHGAVFGGGTKLTVLGQ |
| | HCDR1 | 1 | GFSFGSHYMS |
| | HCDR2 | 3 | NINQIGYSSYYGESVKG |
| | HCDR3 | 6 | GYSDEFATRSGLDV |
| | LCDR1 | 10 | SGSSSNIGSNYVY |
| | LCDR2 | 11 | RNNQRPS |
| | LCDR3 | 13 | AGWSRELHGAV |
| CD3- | VH | 16 | EVQLVESGGGLVQPGGSLRLSCAASGFSFGSHYMSWVRQAPG |
| MAB_{deimm}_4 | | | KGLEWVANINQIGYSSYYGESVKGRFTISRDNAKNSLYLQMNSL |
| | | | RAEDTAVYYCARGYSEEFAHRSGLDVWGQGTLVTVSS |
| | VL | 21 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYY |
| | | | CAGWSRELHGAVFGGGTKLTVLGQ |
| | HCDR1 | 1 | GFSFGSHYMS |
| | HCDR2 | 3 | NINQIGYSSYYGESVKG |
| | HCDR3 | 7 | GYSEEFAHRSGLDV |
| | LCDR1 | 10 | SGSSSNIGSNYVY |
| | LCDR2 | 11 | RNNQRPS |
| | LCDR3 | 13 | AGWSRELHGAV |
| CD3- | VH | 17 | EVQLVESGGGLVQPGGSLRLSCAASGFSFGSHYMSWVRQAPG |
| MAB_{deimm}_5 | | | KGLEWVANINQIGYSSYYGESVKGRFTISRDNAKNSLYLQMNSL |
| | | | RAEDTAVYYCARGYSDEFAKRSGLDVWGQGTLVTVSS |
| | VL | 21 | QSVLTQPPSASGTPGQRVTISCSGSSSNIGSNYVYWYQQLPGTA |
| | | | PKLLIYRNNQRPSGVPDRFSGSKSGTSASLAISGLRSEDEADYY |
| | | | CAGWSRELHGAVFGGGTKLTVLGQ |
| | HCDR1 | 1 | GFSFGSHYMS |
| | HCDR2 | 3 | NINQIGYSSYYGESVKG |
| | HCDR3 | 8 | GYSDEFAKRSGLDV |
| | LCDR1 | 10 | SGSSSNIGSNYVY |
| | LCDR2 | 11 | RNNQRPS |
| | LCDR3 | 13 | AGWSRELHGAV |

TABLE 8
Amino acid sequences of peptide linkers used in
the bispecific 2 + 1 Fab₂-Fv-Fc antibodies
according to Example 3.

| Linker | Sequence [aa] | SEQ ID NO: |
|---|---|---|
| (GGS)₃ | GGSGGSGGS | 30 |
| (GGGGS)₄/(G₄S)₄ | GGGGGGGGSGGGGSGGGGS | 31 |
| 20 aa-PAH | AHPAAPAPAHPAAPAPAHGH | 32 |
| 20 aa-PAPDA | AQPAAPAPDAHEAPAPAQGS | 33 |
| 20 aa-Cλ | PKAAPSVTLFPPSSEELQAN | 34 |
| 20 aa-CH1 | ASTKGPSVFPLAPSSKSTSG | 35 |
| 5 aa-Cλ | PKAAP | 36 |
| 6 aa-CH1 | ASTKGP | 37 |
| Hinge_{trunc.} | DKTHTCPPCP | 38 |

TABLE 9
Amino acid sequences of peptide linkers used in
the bispecific 2 + 1 Fab2-Fc-scFv antibody
format according Example 1.

| | | SEQ |
|---|---|---|
| | | ID |
| Linker | Sequence [aa] | NO: |
| 10 aa-PAPAE [Linker to | AQPAAPAPAE | 39 |
| connect one Fc-region | | |
| subunit to scFv] | | |
| 20 aa-PAPNA [scFv-Linker] | AQPAAPAPNAHEAPAPAQGS | 40 |

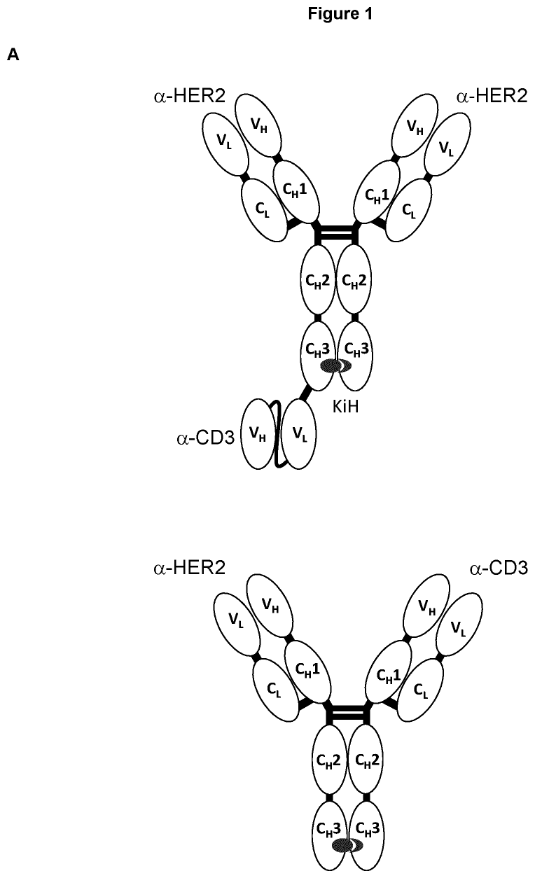

TABLE 10
Amino acid sequences of exemplary polypeptides forming the trivalent bispecific Fab₂-
Fc-scFv antibody format according to Example 1.2 and as shown in FIG. 1A with
bivalent binding to HER2 and monovalent binding to CD3. Bispecific antibody
Bisslg_08_#1 comprises the antibody variable domains of CD3-MABopt_VL whereas
Bisslg_08 #2 comprises the antibody variable domains of MABopt_VH#1.

| | | SEQ | |
|---|---|---|---|
| | | ID | |
| Antibody | | NO | [aa] |
| Bisslg_08_#1 | Fab HC_{(Trast)} - | 49 | QVQLVESGGGLVQPGGSLRLSCAASGFNIKDTYIHWVR |
| | CH2_{AEASS} - | | QAPGKGLEWVARIYPTNGYTRYADSVKGRFTISADTSKN |
| | CH3_{Knob} - Linker - | | TAYLQMNSLRAEDTAVYYCSRWGGDGFYAMDYWGQG |
| | scFV_{(CD3-MABopt}_VL) | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKRVEPKSCDKTHTCP |
| | | | PCPAPEAEGAPSVFLFPPKPKDTLMISRTPEVTCVVVDV |
| | | | SHEDPEVKFNWYVDGVEVHNAKTKPREEQYNSTYRVVS |
| | | | VLTVLHQDWLNGKEYKCKVSNKALPSSIEKTISKAKGQP |
| | | | REPQVYTLPPCREEMTKNQVSLWCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGAQPAAPAPAEQ |
| | | | SVLTQPPSASGTPGQRVTISCSGSSSNIGSNYVYWYQQL |
| | | | PGTAPKLLIYRNNQRPSGVPDRFSGSKSGTSASLAISGL |
| | | | RSEDEADYYCAGWSRSLHGAVFGGGTKLTVLGQAQPA |
| | | | APAPNAHEAPAPAQGSEVQLVESGGGLVQPGGSLRLSC |
| | | | AASGFTFSSYWMSWVRQAPGKGLEWVANIKQDGSEKY |
| | | | YVDSVKGRFTISRDNAKNSLYLQMNSLRAEDTAVYYCAR |
| | | | GYSAEFAHRSGLDVWGQGTLVTVSS |
| | Fab-HC_{(Trast)} - | 50 | QVQLVESGGGLVQPGGSLRLSCAASGFNIKDTYIHWVR |
| | CH2_{AEASS} - CH3_{Hole} | | QAPGKGLEWVARIYPTNGYTRYADSVKGRFTISADTSKN |
| | | | TAYLQMNSLRAEDTAVYYCSRWGGDGFYAMDYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKRVEPKSCDKTHTCP |
| | | | PCPAPEAEGAPSVFLFPPKPKDTLMISRTPEVTCVVVDV |
| | | | SHEDPEVKFNWYVDGVEVHNAKTKPREEQYNSTYRVVS |
| | | | VLTVLHQDWLNGKEYKCKVSNKALPSSIEKTISKAKGQP |
| | | | REPQVCTLPPSREEMTKNQVSLSCAVKGFYPSDIAVEW |
| | | | ESNGQPENNYKTTPPVLDSDGSFFLVSKLTVDKSRWQQ |
| | | | GNVFSCSVMHEALHNHYTQKSLSLSPGK |
| | Fab-LC_{(Trast.)} | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASWVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |
| Bisslg_08_#2 | Fab-HC_{(Trast)} - | 52 | QVQLVESGGGLVQPGGSLRLSCAASGFNIKDTYIHWVR |
| | CH2_{AEASS} - | | QAPGKGLEWVARIYPTNGYTRYADSVKGRFTISADTSKN |
| | CH3_{Knob} - Linker - | | TAYLQMNSLRAEDTAVYYCSRWGGDGFYAMDYWGQG |
| | scFV_{(CD3-MABopt}_VH#1) | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKRVEPKSCDKTHTCP |
| | | | PCPAPEAEGAPSVFLFPPKPKDTLMISRTPEVTCVVVDV |
| | | | SHEDPEVKFNWYVDGVEVHNAKTKPREEQYNSTYRVVS |
| | | | VLTVLHQDWLNGKEYKCKVSNKALPSSIEKTISKAKGQP |
| | | | REPQVYTLPPCREEMTKNQVSLWCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGAQPAAPAPAEQ |
| | | | SVLTQPPSASGTPGQRVTISCSGSSSNIGSNYVYWYQQL |
| | | | PGTAPKLLIYRNNQRPSGVPDRFSGSKSGTSASLAISGL |
| | | | RSEDEADYYCAAWDHHRSGAVFGGGTKLTVLGQAQPA |
| | | | APAPNAHEAPAPAQGSEVQLVESGGGLVQPGGSLRLSC |
| | | | AASGFSFGSHYMSWVRQAPGKGLEWVANINQIGYSSYY |
| | | | VESVKGRFTISRDNAKNSLYLQMNSLRAEDTAVYYCARG |
| | | | YSAEFAHRSGLDVWGQGTLVTVSS |
| | Fab-HC_{(Trast)}- | 50 | QVQLVESGGGLVQPGGSLRLSCAASGFNIKDTYIHWVR |
| | CH2_{AEASS} - CH3_{Hole} | | QAPGKGLEWVARIYPTNGYTRYADSVKGRFTISADTSKN |
| | | | TAYLQMNSLRAEDTAVYYCSRWGGDGFYAMDYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKRVEPKSCDKTHTCP |
| | | | PCPAPEAEGAPSVFLFPPKPKDTLMISRTPEVTCVVVDV |
| | | | SHEDPEVKFNWYVDGVEVHNAKTKPREEQYNSTYRVVS |
| | | | VLTVLHQDWLNGKEYKCKVSNKALPSSIEKTISKAKGQP |
| | | | REPQVCTLPPSREEMTKNQVSLSCAVKGFYPSDIAVEW |
| | | | ESNGQPENNYKTTPPVLDSDGSFFLVSKLTVDKSRWQQ |
| | | | GNVFSCSVMHEALHNHYTQKSLSLSPGK |
| | Fab-LC_{(Trast.)} | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |

TABLE 11
Amino acid sequences of the polypeptides forming the source IgG1 molecules of
Example 2 with specificity for HER2 (IgG1#) and CD3 (IgGopt_cc) used to generate the
bispecific antibody Bisslg_18_opt_cc# via Fab arm exchange. The CD3 specific IgG
comprises the VL of CD3-MABopt_VL and the VH of MABopt_VH#1 and as such
reflects cross-clone antibody CD3-MABopt_cc.

| | | SEQ | |
|---|---|---|---|
| | | ID | |
| Antibody | | NO: | [aa] |
| IgG#1 | VH_{(Trast.)} - CH1 - | 53 | QVQLVESGGGLVQPGGSLRLSCAASGFNIKDTYIHWVRQ |
| | CH2_{(AEASS)} - | | APGKGLEWVARIYPTNGYTRYADSVKGRFTISADTSKNTA |
| | CH3_{(K409R)} | | YLQMNSLRAEDTAVYYCSRWGGDGFYAMDYWGQGTLVT |
| | | | VSSASTKGPSVFPLAPSSKSTSGGTAALGCLVKDYFPEPV |
| | | | TVSWNSGALTSGVHTFPAVLQSSGLYSLSSVVTVPSSSLG |
| | | | TQTYICNVNHKPSNTKVDKRVEPKSCDKTHTCPPCPAPEA |
| | | | EGAPSVFLFPPKPKDTLMISRTPEVTCVVVDVSHEDPEVK |
| | | | FNWYVDGVEVHNAKTKPREEQYNSTYRVVSVLTVLHQD |
| | | | WLNGKEYKCKVSNKALPSSIEKTISKAKGQPREPQVYTLP |
| | | | PSREEMTKNQVSLTCLVKGFYPSDIAVEWESNGQPENNY |
| | | | KTTPPVLDSDGSFFLYSRLTVDKSRWQQGNVFSCSVMHE |
| | | | ALHNHYTQKSLSLSPGK |
| | VL_{(Trast.)} - CL | 51 | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPPS |
| | | | DEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| IgGopt_cc | VH(_{CD3-MABopt}_VH#1) - | 54 | EVQLVESGGGLVQPGGSLRLSCAASGFSFGSHYMSWVR |
| | CH1 - CH2_{(AEASS)} - | | QAPGKGLEWVANINQIGYSSYYVESVKGRFTISRDNAKNS |
| | CH3_{(K409L)} | | LYLQMNSLRAEDTAVYYCARGYSAEFAHRSGLDVWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKRVEPKSCDKTHTCPPCP |
| | | | APEAEGAPSVFLFPPKPKDTLMISRTPEVTCVVVDVSHED |
| | | | PEVKFNWYVDGVEVHNAKTKPREEQYNSTYRVVSVLTVL |
| | | | HQDWLNGKEYKCKVSNKALPSSIEKTISKAKGQPREPQVY |
| | | | TLPPSREEMTKNQVSLTCLVKGFYPSDIAVEWESNGQPE |
| | | | NNYKTTPPVLDSDGSFLLYSKLTVDKSRWQQGNVFSCSV |
| | | | MHEALHNHYTQKSLSLSPGK |
| | VL_{(CD3-MABopt}_VL) - | 55 | QSVLTQPPSASGTPGQRVTISCSGSSSNIGSNYVYWYQQ |
| | CL | | LPGTAPKLLIYRNNQRPSGVPDRFSGSKSGTSASLAISGLR |
| | | | SEDEADYYCAGWSRSLHGAVFGGGTKLTVLGQPKAAPSV |
| | | | TLFPPSSEELQANKATLVCLISDFYPGAVTVAWKADSSPV |
| | | | KAGVETTTPSKQSNNKYAASSYLSLTPEQWKSHRSYSCQ |
| | | | VTHEGSTVEKTVAPTECS |

TABLE 12
Amino acid sequences of polypeptides forming the bivalent bispecific 1 + 1 antibody
Bisslg_18_opt_cc# (after Fab arm of exchange of lgG#1 and IgGopt_cc) according to
Table 11, Example 2 and as shown in Figure 1B.

| | | SEQ | |
|---|---|---|---|
| | | ID | |
| Bispecific antibody | | NO: | [aa] |
| Bisslg_18_opt_ | VH_{(Trast.)} - CH1 - | 53 | QVQLVESGGGLVQPGGSLRLSCAASGFNIKDTYIHWVRQ |
| cc# | CH2_{(AEASS)} - | | APGKGLEWVARIYPTNGYTRYADSVKGRFTISADTSKNTA |
| | CH3_{(K409R)} | | YLQMNSLRAEDTAVYYCSRWGGDGFYAMDYWGQGTLVT |
| | | | VSSASTKGPSVFPLAPSSKSTSGGTAALGCLVKDYFPEPV |
| | | | TVSWNSGALTSGVHTFPAVLQSSGLYSLSSVVTVPSSSLG |
| | | | TQTYICNVNHKPSNTKVDKRVEPKSCDKTHTCPPCPAPEA |
| | | | EGAPSVFLFPPKPKDTLMISRTPEVTCVVVDVSHEDPEVK |
| | | | FNWYVDGVEVHNAKTKPREEQYNSTYRVVSVLTVLHQD |
| | | | WLNGKEYKCKVSNKALPSSIEKTISKAKGQPREPQVYTLP |
| | | | PSREEMTKNQVSLTCLVKGFYPSDIAVEWESNGQPENNY |
| | | | KTTPPVLDSDGSFFLYSRLTVDKSRWQQGNVFSCSVMHE |
| | | | ALHNHYTQKSLSLSPGK |
| | VL_{(Trast.)} - CL | 51 | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPPS |
| | | | DEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| | VH_{(CD3-MABopt}_VH#1) - | 54 | EVQLVESGGGLVQPGGSLRLSCAASGFSFGSHYMSWVR |
| | CH1 - CH2_{(AEASS)} - | | QAPGKGLEWVANINQIGYSSYYVESVKGRFTISRDNAKNS |
| | CH3_{(K409L)} | | LYLQMNSLRAEDTAVYYCARGYSAEFAHRSGLDVWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKRVEPKSCDKTHTCPPCP |
| | | | APEAEGAPSVFLFPPKPKDTLMISRTPEVTCVVVDVSHED |
| | | | PEVKFNWYVDGVEVHNAKTKPREEQYNSTYRVVSVLTVL |
| | | | HQDWLNGKEYKCKVSNKALPSSIEKTISKAKGQPREPQVY |
| | | | TLPPSREEMTKNQVSLTCLVKGFYPSDIAVEWESNGQPE |
| | | | NNYKTTPPVLDSDGSFLLYSKLTVDKSRWQQGNVFSCSV |
| | | | MHEALHNHYTQKSLSLSPGK |
| | VL_{(CD3-MABopt}_VL) - | 55 | QSVLTQPPSASGTPGQRVTISCSGSSSNIGSNYVYWYQQ |
| | CL | | LPGTAPKLLIYRNNQRPSGVPDRFSGSKSGTSASLAISGLR |
| | | | SEDEADYYCAGWSRSLHGAVFGGGTKLTVLGQPKAAPSV |
| | | | TLFPPSSEELQANKATLVCLISDFYPGAVTVAWKADSSPV |
| | | | KAGVETTTPSKQSNNKYAASSYLSLTPEQWKSHRSYSCQ |
| | | | VTHEGSTVEKTVAPTECS |

TABLE 13
Amino acid sequences of the polypeptides forming the bispecific antibodies according
to Example 3 (and as shown in FIG. 2) with improved peptide Linker Combinations
as listed in Table 23. All bispecific antibodies employ the VH and VL of the cross-
cloned antibody CD3-MABopt_cc.

| | | SEQ | |
|---|---|---|---|
| | | ID | |
| Bispecific antibody | | NO: | [aa] |
| Bisslg_21#P | VH_{(a-HER2)} - CH1 - | 56 | QVQLVESGGGLVQPGGSLRLSCAASGFNIKDTYIHWVRQA |
| Linker | Linker_{(GGS)3} - | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| P | Linker_{(SaaCLλ)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3_{(knob)} | | YICNVNHKPSNTKVDKRVEPKSCGGSGGSGGSQSVLTQP |
| | | | PSASGTPGQRVTISCSGSSSNIGSNYVYWYQQLPGTAPKL |
| | | | LIYRNNQRPSGVPDRFSGSKSGTSASLAISGLRSEDEADYY |
| | | | CAGWSRSLHGAVFGGGTKLTVLGQPKAAPDKTHTCPPCP |
| | | | APEAEGAPSVFLFPPKPKDTLMISRTPEVTCVVVDVSHEDP |
| | | | EVKFNWYVDGVEVHNAKTKPREEQYNSTYRVVSVLTVLH |
| | | | QDWLNGKEYKCKVSNKALPSSIEKTISKAKGQPREPQVYTL |
| | | | PPCREEMTKNQVSLWCLVKGFYPSDIAVEWESNGQPENN |
| | | | YKTTPPVLDSDGSFFLYSKLTVDKSRWQQGNVFSCSVMH |
| | | | EALHNHYTQKSLSLSPGK |
| | VH_{(Trast.)} - CH1 - | 57 | QVQLVESGGGLVQPGGSLRLSCAASGFNIKDTYIHWVRQA |
| | Linker_{(GGS)3} - | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(6aaCH1)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCGGSGGSGGSEVQLVES |
| | | | GGGLVQPGGSLRLSCAASGFSFGSHYMSWVRQAPGKGL |
| | | | EWVANINQIGYSSYYVESVKGRFTISRDNAKNSLYLQMNSL |
| | | | RAEDTAVYYCARGYSAEFAHRSGLDVWGQGTLVTVSSAS |
| | | | TKGPDKTHTCPPCPAPEAEGAPSVFLFPPKPKDTLMISRTP |
| | | | EVTCVVVDVSHEDPEVKFNWYVDGVEVHNAKTKPREEQY |
| | | | NSTYRVVSVLTVLHQDWLNGKEYKCKVSNKALPSSIEKTIS |
| | | | KAKGQPREPQVCTLPPSREEMTKNQVSLSCAVKGFYPSDI |
| | | | AVEWESNGQPENNYKTTPPVLDSDGSFFLVSKLTVDKSR |
| | | | WQQGNVFSCSVMHEALHNHYTQKSLSLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLQPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |
| Bisslg_21#1 | VH_{(Trast.)} - CH1 - | 58 | QVQLVESGGGLVQPGGSLRLSCAASGFNIKDTYIHWVRQ |
| Linker | Linker_{(G4S)4} - | | APGKGLEWVARIYPTNGYTRYADSVKGRFTISADTSKNTA |
| Combination | VL_{(a-CD3)} - | | YLQMNSLRAEDTAVYYCSRWGGDGFYAMDYWGQGTLVT |
| 1 | Linker_{(5aaCLλ)} - | | VSSASTKGPSVFPLAPSSKSTSGGTAALGCLVKDYFPEPV |
| | CH2_{(AEASS)} - | | TVSWNSGALTSGVHTFPAVLQSSGLYSLSSVVTVPSSSLG |
| | CH3_{(knob)} | | TQTYICNVNHKPSNTKVDKRVEPKSCGGGGSGGGGSGG |
| | | | GGSGGGGSQSVLTQPPSASGTPGQRVTISCSGSSSNIGS |
| | | | NYVYWYQQLPGTAPKLLIYRNNQRPSGVPDRFSGSKSGT |
| | | | SASLAISGLRSEDEADYYCAGWSRSLHGAVFGGGTKLTVL |
| | | | GQPKAAPDKTHTCPPCPAPEAEGAPSVFLFPPKPKDTLMI |
| | | | SRTPEVTCVVVDVSHEDPEVKFNWYVDGVEVHNAKTKPR |
| | | | EEQYNSTYRVVSVLTVLHQDWLNGKEYKCKVSNKALPSSI |
| | | | EKTISKAKGQPREPQVYTLPPCREEMTKNQVSLWCLVKG |
| | | | FYPSDIAVEWESNGQPENNYKTTPPVLDSDGSFFLYSKLT |
| | | | VDKSRWQQGNVFSCSVMHEALHNHYTQKSLSLSPGK |
| | VH_{(Trast.)} - CH1 - | 59 | QVQLVESGGGLVQPGGSLRLSCAASGFNIKDTYIHWVRQ |
| | Linker_{(G4S)4} - | | APGKGLEWVARIYPTNGYTRYADSVKGRFTISADTSKNTA |
| | VH_{(a-CD3)} - | | YLQMNSLRAEDTAVYYCSRWGGDGFYAMDYWGQGTLVT |
| | Linker _{(6aaCH1)} - | | VSSASTKGPSVFPLAPSSKSTSGGTAALGCLVKDYFPEPV |
| | CH2_{(AEASS)} - | | TVSWNSGALTSGVHTFPAVLQSSGLYSLSSVVTVPSSSLG |
| | CH3₍ₕₒₗₑ₎ | | TQTYICNVNHKPSNTKVDKRVEPKSCGGGGSGGGGSGG |
| | | | GGSGGGGSEVQLVESGGGLVQPGGSLRLSCAASGFSFG |
| | | | SHYMSWVRQAPGKGLEWVANINQIGYSSYYVESVKGRFT |
| | | | ISRDNAKNSLYLQMNSLRAEDTAVYYCARGYSAEFAHRS |
| | | | GLDVWGQGTLVTVSSASTKGPDKTHTCPPCPAPEAEGAP |
| | | | SVFLFPPKPKDTLMISRTPEVTCVVVDVSHEDPEVKFNWY |
| | | | VDGVEVHNAKTKPREEQYNSTYRVVSVLTVLHQDWLNGK |
| | | | EYKCKVSNKALPSSIEKTISKAKGQPREPQVCTLPPSREE |
| | | | MTKNQVSLSCAVKGFYPSDIAVEWESNGQPENNYKTTPP |
| | | | VLDSDGSFFLVSKLTVDKSRWQQGNVFSCSVMHEALHNH |
| | | | YTQKSLSLSPGK |
| | VL_{(Trast.)} - CL | 51 | DIQMTQSPSSLSASVGDRVTITCRASQDVNTAVAWYQQK |
| | | | PGKAPKLLIYSASFLYSGVPSRFSGSRSGTDFTLTISSLQP |
| | | | EDFATYYCQQHYTTPPTFGQGTKVEIKRTVAAPSVFIFPPS |
| | | | DEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| Bisslg_21#2 | VH_{(Trast.)} - CH1 - | 60 | QVQLVESGGGLVQPGGSLRLSCAASGFNIKDTYIHWVRQA |
| Linker | Linker_{(G4S)4} | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| 2 | Linker_{(20 aaPAPDA)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3_{(knob)} | | YICNVNHKPSNTKVDKRVEPKSCGGGGSGGGGSGGGGS |
| | | | GGGGSQSVLTQPPSASGTPGQRVTISCSGSSSNIGSNYVY |
| | | | WYQQLPGTAPKLLIYRNNQRPSGVPDRFSGSKSGTSASLA |
| | | | ISGLRSEDEADYYCAGWSRSLHGAVFGGGTKLTVLGQAQ |
| | | | PAAPAPDAHEAPAPAQGSDKTHTCPPCPAPEAEGAPSVFL |
| | | | FPPKPKDTLMISRTPEVTCVVVDVSHEDPEVKFNWYVDGV |
| | | | EVHNAKTKPREEQYNSTYRVVSVLTVLHQDWLNGKEYKC |
| | | | KVSNKALPSSIEKTISKAKGQPREPQVYTLPPCREEMTKNQ |
| | | | VSLWCLVKGFYPSDIAVEWESNGQPENNYKTTPPVLDSDG |
| | | | SFFLYSKLTVDKSRWQQGNVFSCSVMHEALHNHYTQKSL |
| | | | SLSPGK |
| | VH_{(Trast.)} - CH1 - | 61 | QVQLVESGGGLVQPGGSLRLSCAASGFNIKDTYIHWVRQA |
| | Linker_{(G4S)4} - | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(20 aaPAPDA)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCGGGGSGGGGGGGGS |
| | | | GGGGSEVQLVESGGGLVQPGGSLRLSCAASGFSFGSHYM |
| | | | SWVRQAPGKGLEWVANINQIGYSSYYVESVKGRFTISRDN |
| | | | AKNSLYLQMNSLRAEDTAVYYCARGYSAEFAHRSGLDVW |
| | | | GQGTLVTVSSAQPAAPAPDAHEAPAPAQGSDKTHTCPPC |
| | | | PAPEAEGAPSVFLFPPKPKDTLMISRTPEVTCVVVDVSHED |
| | | | PEVKFNWYVDGVEVHNAKTKPREEQYNSTYRVVSVLTVLH |
| | | | QDWLNGKEYKCKVSNKALPSSIEKTISKAKGQPREPQVCT |
| | | | LPPSREEMTKNQVSLSCAVKGFYPSDIAVEWESNGQPENN |
| | | | YKTTPPVLDSDGSFFLVSKLTVDKSRWQQGNVFSCSVMH |
| | | | EALHNHYTQKSLSLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLQPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |
| Bisslg_21#3 | VH_{(Trast.)} - CH1 - | 62 | QVQLVESGGGLVQPGGSLRLSCAASGFNIKDTYIHWVRQA |
| Linker | Linker_{(G4S)4} - | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| 3 | Linker_{(20 aaCLλ)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3_{(knob)} | | YICNVNHKPSNTKVDKRVEPKSCGGGGSGGGGSGGGGS |
| | | | GGGGSQSVLTQPPSASGTPGQRVTISCSGSSSNIGSNYVY |
| | | | WYQQLPGTAPKLLIYRNNQRPSGVPDRFSGSKSGTSASLA |
| | | | ISGLRSEDEADYYCAGWSRSLHGAVFGGGTKLTVLGQPKA |
| | | | APSVTLFPPSSEELQANDKTHTCPPCPAPEAEGAPSVFLFP |
| | | | PKPKDTLMISRTPEVTCVVVDVSHEDPEVKFNWYVDGVEV |
| | | | HNAKTKPREEQYNSTYRWSVLTVLHQDWLNGKEYKCKV |
| | | | SNKALPSSIEKTISKAKGQPREPQVYTLPPCREEMTKNQVS |
| | | | LWCLVKGFYPSDIAVEWESNGQPENNYKTTPPVLDSDGSF |
| | | | FLYSKLTVDKSRWQQGNVFSCSVMHEALHNHYTQKSLSLS |
| | | | PGK |
| | VH_{(Trast.)} - CH1 - | 63 | QVQLVESGGGLVQPGGSLRLSCAASGFNIKDTYIHWVRQA |
| | Linker_{(G4S)4} | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(20 aaCH1)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCGGGGSGGGGSGGGGS |
| | | | GGGGSEVQLVESGGGLVQPGGSLRLSCAASGFSFGSHYM |
| | | | SWVRQAPGKGLEWVANINQIGYSSYYVESVKGRFTISRDN |
| | | | AKNSLYLQMNSLRAEDTAVYYCARGYSAEFAHRSGLDVW |
| | | | GQGTLVTVSSASTKGPSVFPLAPSSKSTSGDKTHTCPPCP |
| | | | APEAEGAPSVFLFPPKPKDTLMISRTPEVTCVVVDVSHEDP |
| | | | EVKFNWYVDGVEVHNAKTKPREEQYNSTYRVVSVLTVLH |
| | | | QDWLNGKEYKCKVSNKALPSSIEKTISKAKGQPREPQVCT |
| | | | LPPSREEMTKNQVSLSCAVKGFYPSDIAVEWESNGQPENN |
| | | | YKTTPPVLDSDGSFFLVSKLTVDKSRWQQGNVFSCSVMH |
| | | | EALHNHYTQKSLSLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLQPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |
| Bisslg_21#4 | VH_{(Trast.)} - CH1 - | 64 | QVQLVESGGGLVQPGGSLRLSCAASGFNIKDTYIHWVRQA |
| Linker | Linker_{(20 aaPAH)} - | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| 4 | Linker_{(20 aaCLλ)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3_{(knob)} | | YICNVNHKPSNTKVDKRVEPKSCAHPAAPAPAHPAAPAPA |
| | | | HGHQSVLTQPPSASGTPGQRVTISCSGSSSNIGSNYVYWY |
| | | | QQLPGTAPKLLIYRNNQRPSGVPDRFSGSKSGTSASLAISG |
| | | | LRSEDEADYYCAGWSRSLHGAVFGGGTKLTVLGQPKAAP |
| | | | SVTLFPPSSEELQANDKTHTCPPCPAPEAEGAPSVFLFPPK |
| | | | PKDTLMISRTPEVTCVVVDVSHEDPEVKFNWYVDGVEVHN |
| | | | AKTKPREEQYNSTYRVVSVLTVLHQDWLNGKEYKCKVSNK |
| | | | ALPSSIEKTISKAKGQPREPQVYTLPPCREEMTKNQVSLWC |
| | | | LVKGFYPSDIAVEWESNGQPENNYKTTPPVLDSDGSFFLY |
| | | | SKLTVDKSRWQQGNVFSCSVMHEALHNHYTQKSLSLSPG |
| | | | K |
| | VH_{(Trast.)} - CH1 - | 65 | QVQLVESGGGLVQPGGSLRLSCAASGFNIKDTYIHWVRQA |
| | Linker _{(20 aaPAH)} - | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(20 aaCH1)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCAHPAAPAPAHPAAPAPA |
| | | | HGHEVQLVESGGGLVQPGGSLRLSCAASGFSFGSHYMS |
| | | | WVRQAPGKGLEWVANINQIGYSSYYVESVKGRFTISRDNA |
| | | | KNSLYLQMNSLRAEDTAVYYCARGYSAEFAHRSGLDVWG |
| | | | QGTLVTVSSASTKGPSVFPLAPSSKSTSGDKTHTCPPCPA |
| | | | PEAEGAPSVFLFPPKPKDTLMISRTPEVTCWVDVSHEDPE |
| | | | VKFNWYVDGVEVHNAKTKPREEQYNSTYRVVSVLTVLHQ |
| | | | DWLNGKEYKCKVSNKALPSSIEKTISKAKGQPREPQVCTLP |
| | | | PSREEMTKNQVSLSCAVKGFYPSDIAVEWESNGQPENNY |
| | | | KTTPPVLDSDGSFFLVSKLTVDKSRWQQGNVFSCSVMHE |
| | | | ALHNHYTQKSLSLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLQPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |
| Bisslg_21#5 | VH_{(Trast.)} - CH1 - | 66 | QVQLVESGGGLVQPGGSLRLSCAASGFNIKDTYIHWVRQA |
| Linker | Linker_{(20 aaPAPDA)} - | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| 5 | Linker_{(20 aaCLλ)} - | | SASTKGPSVFPLAPSSKSTSGGTAALGCLVKDYFPEPVTVS |
| | CH2_{(AEASS)} | | WNSGALTSGVHTFPAVLQSSGLYSLSSVVTVPSSSLGTQT |
| | CH3_{(knob)} | | YICNVNHKPSNTKVDKRVEPKSCAQPAAPAPDAHEAPAPA |
| | | | QGSQSVLTQPPSASGTPGQRVTISCSGSSSNIGSNYVYWY |
| | | | QQLPGTAPKLLIYRNNQRPSGVPDRFSGSKSGTSASLAISG |
| | | | LRSEDEADYYCAGWSRSLHGAVFGGGTKLTVLGQPKAAP |
| | | | SVTLFPPSSEELQANDKTHTCPPCPAPEAEGAPSVFLFPPK |
| | | | PKDTLMISRTPEVTCVVVDVSHEDPEVKFNWYVDGVEVHN |
| | | | AKTKPREEQYNSTYRVVSVLTVLHQDWLNGKEYKCKVSNK |
| | | | ALPSSIEKTISKAKGQPREPQVYTLPPCREEMTKNQVSLWC |
| | | | LVKGFYPSDIAVEWESNGQPENNYKTTPPVLDSDGSFFLY |
| | | | SKLTVDKSRWQQGNVFSCSVMHEALHNHYTQKSLSLSPG |
| | | | K |
| | VH_{(Trast.)} - CH1 - | 67 | QVQLVESGGGLVQPGGSLRLSCAASGFNIKDTYIHWVRQA |
| | Linker_{(20 aaPAPDA)} - | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(20 aaCH1)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCAQPAAPAPDAHEAPAPA |
| | | | QGSEVQLVESGGGLVQPGGSLRLSCAASGFSFGSHYMS |
| | | | WVRQAPGKGLEWVANINQIGYSSYYVESVKGRFTISRDNA |
| | | | KNSLYLQMNSLRAEDTAVYYCARGYSAEFAHRSGLDVWG |
| | | | QGTLVTVSSASTKGPSVFPLAPSSKSTSGDKTHTCPPCPA |
| | | | PEAEGAPSVFLFPPKPKDTLMISRTPEVTCVVVDVSHEDPE |
| | | | VKFNWYVDGVEVHNAKTKPREEQYNSTYRVVSVLTVLHQ |
| | | | DWLNGKEYKCKVSNKALPSSIEKTISKAKGQPREPQVCTLP |
| | | | PSREEMTKNQVSLSCAVKGFYPSDIAVEWESNGQPENNY |
| | | | KTTPPVLDSDGSFFLVSKLTVDKSRWQQGNVFSCSVMHE |
| | | | ALHNHYTQKSLSLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLOPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |
| Bisslg_21#6 | VH_{(Trast.)} - CH1 - | 68 | QVQLVESGGGLVQPGGSLRLSCAASGFNIKDTYIHWVRQA |
| Linker | Linker_{(GGS)3} - | | PGKGLEWVARIYPTNGYTRYADSVKGRFTISADTSKNTAYL |
| Combination | VL_{(a-CD3)} - | | QMNSLRAEDTAVYYCSRWGGDGFYAMDYWGQGTLVTVS |
| 6 | Linker_{(20 aaCLλ)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3_{(knob)} | | YICNVNHKPSNTKVDKRVEPKSCGGSGGSGGSQSVLTQP |
| | | | PSASGTPGQRVTISCSGSSSNIGSNYVYWYQQLPGTAPKL |
| | | | LIYRNNQRPSGVPDRFSGSKSGTSASLAISGLRSEDEADYY |
| | | | CAGWSRSLHGAVFGGGTKLTVLGQPKAAPSVTLFPPSSEE |
| | | | LQANDKTHTCPPCPAPEAEGAPSVFLFPPKPKDTLMISRTP |
| | | | EVTCVWVDVSHEDPEVKFNWYVDGVEVHNAKTKPREEQY |
| | | | NSTYRVVSVLTVLHQDWLNGKEYKCKVSNKALPSSIEKTIS |
| | | | KAKGQPREPQVYTLPPCREEMTKNQVSLWCLVKGFYPSDI |
| | | | AVEWESNGQPENNYKTTPPVLDSDGSFFLYSKLTVDKSR |
| | | | WQQGNVFSCSVMHEALHNHYTQKSLSLSPGK |
| | VH_{(Trast.)} - CH1 - | 69 | QVQLVESGGGLVQPGGSLRLSCAASGFNIKDTYIHWVRQA |
| | Linker_{(GGS)3)} - | | PGKGLEWVARIYPTNGYTRYADSVKGRFTISADTSKNTAYL |
| | VH_{(a-CD3)} - | | QMNSLRAEDTAVYYCSRWGGDGFYAMDYWGQGTLVTVS |
| | Linker_{(20 aaCH1)} - | | SASTKGPSVFPLAPSSKSTSGGTAALGCLVKDYFPEPVTVS |
| | CH2_{(AEASS)} - | | WNSGALTSGVHTFPAVLQSSGLYSLSSVVTVPSSSLGTQT |
| | CH3₍ₕₒₗₑ₎ | | YICNVNHKPSNTKVDKRVEPKSCGGSGGSGGSEVQLVES |
| | | | GGGLVQPGGSLRLSCAASGFSFGSHYMSWVRQAPGKGL |
| | | | EWVANINQIGYSSYYVESVKGRFTISRDNAKNSLYLQMNSL |
| | | | RAEDTAVYYCARGYSAEFAHRSGLDVWGQGTLVTVSSAS |
| | | | TKGPSVFPLAPSSKSTSGDKTHTCPPCPAPEAEGAPSVFL |
| | | | FPPKPKDTLMISRTPEVTCVVVDVSHEDPEVKFNWYVDGV |
| | | | EVHNAKTKPREEQYNSTYRVVSVLTVLHQDWLNGKEYKC |
| | | | KVSNKALPSSIEKTISKAKGQPREPQVCTLPPSREEMTKNQ |
| | | | VSLSCAVKGFYPSDIAVEWESNGQPENNYKTTPPVLDSDG |
| | | | SFFLVSKLTVDKSRWQQGNVFSCSVMHEALHNHYTQKSL |
| | | | SLSPGK |
| | VL_{(Trast.)} - CL | 51 | DIQMTQSPSSLSASVGDRVTITCRASQDVNTAVAWYQQKP |
| | | | GKAPKLLIYSASFLYSGVPSRFSGSRSGTDFTLTISSLQPED |
| | | | FATYYCQQHYTTPPTFGQGTKVEIKRTVAAPSVFIFPPSDE |
| | | | QLKSGTASVVCLLNNFYPREAKVQWKVDNALQSGNSQES |
| | | | VTEQDSKDSTYSLSSTLTLSKADYEKHKVYACEVTHQGLS |
| | | | SPVTKSFNRGEC |

TABLE 14
Amino acid sequences of the polypeptides forming the trivalent bispecific antibodies
according to Example 4 employing the preferred deimmunized CD3 specific binding
antibodies according the present disclosure.

| | | SEQ | |
|---|---|---|---|
| | | ID | |
| Bispecific antibody | | NO: | [aa] |
| Bisslg_21# | VH_{(Trast.)} - CH1 - | 70 | QVQLVESGGGLVQPGGSLRLSCAASGFNIKDTYIHWVR |
| CD3-MAB_{deimm}_1 | Linker_{(20 aaPAPDA)} - | | QAPGKGLEWVARIYPTNGYTRYADSVKGRFTISADTSKN |
| | VL_{(CD3-MABdeimm}_1) - | | TAYLQMNSLRAEDTAVYYCSRWGGDGFYAMDYWGQG |
| | Linker_{(20 aaCLλ)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSVVTV |
| | CH3_{(knob)} | | PSSSLGTQTYICNVNHKPSNTKVDKRVEPKSCAQPAAPA |
| | | | PDAHEAPAPAQGSQSVLTQPPSASGTPGQRVTISCSGS |
| | | | SSNIGSNYVYWYQQLPGTAPKLLIYRNNQRPSGVPDRFS |
| | | | GSKSGTSASLAISGLRSEDEADYYCAGWSRELHGAVFG |
| | | | GGTKLTVLGQPKAAPSVTLFPPSSEELQANDKTHTCPPC |
| | | | PAPEAEGAPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPSSIEKTISKAKGQPRE |
| | | | PQVYTLPPCREEMTKNQVSLWCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | VH_{(Trast.)} - CH1 - | 71 | QVQLVESGGGLVQPGGSLRLSCAASGFNIKDTYIHWVR |
| | Linker_{(20 aaPAPDA)} - | | QAPGKGLEWVARIYPTNGYTRYADSVKGRFTISADTSKN |
| | VH_{(CD3-MABdeimm}_1) - | | TAYLQMNSLRAEDTAVYYCSRWGGDGFYAMDYWGQG |
| | Linker_{(20 aaCH1)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSWVTV |
| | CH3₍ₕₒₗₑ₎ | | PSSSLGTQTYICNVNHKPSNTKVDKRVEPKSCAQPAAPA |
| | | | PDAHEAPAPAQGSEVQLVESGGGLVQPGGSLRLSCAAS |
| | | | GFSFGSHYMSWVRQAPGKGLEWVANINQIGYSSYYEES |
| | | | VKGRFTISRDNAKNSLYLQMNSLRAEDTAVYYCARGYSE |
| | | | EFAHRSGLDVWGQGTLVTVSSASTKGPSVFPLAPSSKS |
| | | | TSGDKTHTCPPCPAPEAEGAPSVFLFPPKPKDTLMISRT |
| | | | PEVTCWVDVSHEDPEVKFNWYVDGVEVHNAKTKPREE |
| | | | QYNSTYRVVSVLTVLHQDWLNGKEYKCKVSNKALPSSIE |
| | | | KTISKAKGQPREPQVCTLPPSREEMTKNQVSLSCAVKGF |
| | | | YPSDIAVEWESNGQPENNYKTTPPVLDSDGSFFLVSKLT |
| | | | VDKSRWQQGNVFSCSVMHEALHNHYTQKSLSLSPGK |
| | VL_{(Trast.)} - CL | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |
| Bisslg_21# | VH_{(Trast.)} - CH1 - | 70 | QVQLVESGGGLVQPGGSLRLSCAASGFNIKDTYIHWVR |
| CD3-MAB_{deimm}_2 | Linker_{(20 aaPAPDA)} - | | QAPGKGLEWVARIYPTNGYTRYADSVKGRFTISADTSKN |
| | VL_{(CD3-MABdeimm}_2) - | | TAYLQMNSLRAEDTAVYYCSRWGGDGFYAMDYWGQG |
| | Linker_{(20 aaCL2λ)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSVVTV |
| | CH3_{(knob)} | | PSSSLGTQTYICNVNHKPSNTKVDKRVEPKSCAQPAAPA |
| | | | PDAHEAPAPAQGSQSVLTQPPSASGTPGQRVTISCSGS |
| | | | SSNIGSNYVYWYQQLPGTAPKLLIYRNNQRPSGVPDRFS |
| | | | GSKSGTSASLAISGLRSEDEADYYCAGWSRELHGAVFG |
| | | | GGTKLTVLGQPKAAPSVTLFPPSSEELQANDKTHTCPPC |
| | | | PAPEAEGAPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPSSIEKTISKAKGQPRE |
| | | | PQVYTLPPCREEMTKNQVSLWCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | VH_{(Trast.)} - CH1 - | 72 | QVQLVESGGGLVQPGGSLRLSCAASGFNIKDTYIHWVR |
| | Linker_{(PAPDA}_20 aa) - | | QAPGKGLEWVARIYPTNGYTRYADSVKGRFTISADTSKN |
| | VH_{(CD3-MABdeimm}_2) - | | TAYLQMNSLRAEDTAVYYCSRWGGDGFYAMDYWGQG |
| | Linker_{(20 aaCH1)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSWTV |
| | CH3₍ₕₒₗₑ₎ | | PSSSLGTQTYICNVNHKPSNTKVDKRVEPKSCAQPAAPA |
| | | | PDAHEAPAPAQGSEVQLVESGGGLVQPGGSLRLSCAAS |
| | | | GFSFGSHYMSWVRQAPGKGLEWVANINQIGYSSYYGES |
| | | | VKGRFTISRDNAKNSLYLQMNSLRAEDTAVYYCARGYSD |
| | | | EFAHRSGLDVWGQGTLVTVSSASTKGPSVFPLAPSSKS |
| | | | TSGDKTHTCPPCPAPEAEGAPSVFLFPPKPKDTLMISRT |
| | | | PEVTCWVDVSHEDPEVKFNWYVDGVEVHNAKTKPREE |
| | | | QYNSTYRVVSVLTVLHQDWLNGKEYKCKVSNKALPSSIE |
| | | | KTISKAKGQPREPQVCTLPPSREEMTKNQVSLSCAVKGF |
| | | | YPSDIAVEWESNGQPENNYKTTPPVLDSDGSFFLVSKLT |
| | | | VDKSRWQQGNVFSCSVMHEALHNHYTQKSLSLSPGK |
| | VL_{(Trast.)} - CL | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |
| Bisslg_21# | VH_{(Trast.)} - CH1 - | 70 | QVQLVESGGGLVQPGGSLRLSCAASGFNIKDTYIHWVR |
| CD3-MAB_{deimm}_3 | Linker_{(20 aaPAPDA)} - | | QAPGKGLEWVARIYPTNGYTRYADSVKGRFTISADTSKN |
| | VL_{(CD3-MABdeimm}_3) - | | TAYLQMNSLRAEDTAVYYCSRWGGDGFYAMDYWGQG |
| | Linker_{(20 aaCLλ)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSWTV |
| | CH3_{(knob)} | | PSSSLGTQTYICNVNHKPSNTKVDKRVEPKSCAQPAAPA |
| | | | PDAHEAPAPAQGSQSVLTQPPSASGTPGQRVTISCSGS |
| | | | SSNIGSNYVYWYQQLPGTAPKLLIYRNNQRPSGVPDRFS |
| | | | GSKSGTSASLAISGLRSEDEADYYCAGWSRELHGAVFG |
| | | | GGTKLTVLGQPKAAPSVTLFPPSSEELQANDKTHTCPPC |
| | | | PAPEAEGAPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPSSIEKTISKAKGQPRE |
| | | | PQVYTLPPCREEMTKNQVSLWCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | VH_{(Trast.)} - CH1 - | 73 | QVQLVESGGGLVQPGGSLRLSCAASGFNIKDTYIHWVR |
| | Linker_{(20 aaPAPDA)} - | | QAPGKGLEWVARIYPTNGYTRYADSVKGRFTISADTSKN |
| | VH_{(CD3-MABdeimm}_3) - | | TAYLQMNSLRAEDTAVYYCSRWGGDGFYAMDYWGQG |
| | Linker_{(20 aaCH1)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSWVTV |
| | CH3₍ₕₒₗₑ₎ | | PSSSLGTQTYICNVNHKPSNTKVDKRVEPKSCAQPAAPA |
| | | | PDAHEAPAPAQGSEVQLVESGGGLVQPGGSLRLSCAAS |
| | | | GFSFGSHYMSWVRQAPGKGLEWVANINQIGYSSYYGES |
| | | | VKGRFTISRDNAKNSLYLQMNSLRAEDTAVYYCARGYSD |
| | | | EFATRSGLDVWGQGTLVTVSSASTKGPSVFPLAPSSKST |
| | | | SGDKTHTCPPCPAPEAEGAPSVFLFPPKPKDTLMISRTP |
| | | | EVTCVVVDVSHEDPEVKFNWYVDGVEVHNAKTKPREEQ |
| | | | YNSTYRVVSVLTVLHQDWLNGKEYKCKVSNKALPSSIEK |
| | | | TISKAKGQPREPQVCTLPPSREEMTKNQVSLSCAVKGFY |
| | | | PSDIAVEWESNGQPENNYKTTPPVLDSDGSFFLVSKLTV |
| | | | DKSRWQQGNVFSCSVMHEALHNHYTQKSLSLSPGK |
| | VL_{(Trast.)} - CL | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |
| Bisslg_21# | VH_{(Trast.)} - CH1 - | 70 | QVQLVESGGGLVQPGGSLRLSCAASGFNIKDTYIHWVR |
| CD3-MAB_{deimm}_4 | Linker_{(20 aaPAPDA)} - | | QAPGKGLEWVARIYPTNGYTRYADSVKGRFTISADTSKN |
| | VL_{(CD3-MABdeimm}_4) - | | TAYLQMNSLRAEDTAVYYCSRWGGDGFYAMDYWGQG |
| | Linker_{(20 aaCLλ)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSWTV |
| | CH3_{(knob)} | | PSSSLGTQTYICNVNHKPSNTKVDKRVEPKSCAQPAAPA |
| | | | PDAHEAPAPAQGSQSVLTQPPSASGTPGQRVTISCSGS |
| | | | SSNIGSNYVYWYQQLPGTAPKLLIYRNNQRPSGVPDRFS |
| | | | GSKSGTSASLAISGLRSEDEADYYCAGWSRELHGAVFG |
| | | | GGTKLTVLGQPKAAPSVTLFPPSSEELQANDKTHTCPPC |
| | | | PAPEAEGAPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPSSIEKTISKAKGQPRE |
| | | | PQVYTLPPCREEMTKNQVSLWCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | VH_{(Trast.)} - CH1 - | 74 | QVQLVESGGGLVQPGGSLRLSCAASGFNIKDTYIHWVR |
| | Linker_{(20 aaPAPDA)} - | | QAPGKGLEWVARIYPTNGYTRYADSVKGRFTISADTSKN |
| | VH_{(CD3-MABdeimm}_4) - | | TAYLQMNSLRAEDTAVYYCSRWGGDGFYAMDYWGQG |
| | Linker_{(20 aaCH1)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSVVTV |
| | CH3₍ₕₒₗₑ₎ | | PSSSLGTQTYICNVNHKPSNTKVDKRVEPKSCAQPAAPA |
| | | | PDAHEAPAPAQGSEVQLVESGGGLVQPGGSLRLSCAAS |
| | | | GFSFGSHYMSWVRQAPGKGLEWVANINQIGYSSYYGES |
| | | | VKGRFTISRDNAKNSLYLQMNSLRAEDTAVYYCARGYSE |
| | | | EFAHRSGLDVWGQGTLVTVSSASTKGPSVFPLAPSSKS |
| | | | TSGDKTHTCPPCPAPEAEGAPSVFLFPPKPKDTLMISRT |
| | | | PEVTCWVDVSHEDPEVKFNWYVDGVEVHNAKTKPREE |
| | | | QYNSTYRVVSVLTVLHQDWLNGKEYKCKVSNKALPSSIE |
| | | | KTISKAKGQPREPQVCTLPPSREEMTKNQVSLSCAVKGF |
| | | | YPSDIAVEWESNGQPENNYKTTPPVLDSDGSFFLVSKLT |
| | | | VDKSRWQQGNVFSCSVMHEALHNHYTQKSLSLSPGK |
| | VL_{(Trast.)} - CL | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |
| Bisslg_21# | VH(_{Trast.)} - CH1 - | 70 | QVQLVESGGGLVQPGGSLRLSCAASGFNIKDTYIHWVR |
| CD3-MAB_{deimm}_5 | Linker_{(20 aaPAPDA)} - | | QAPGKGLEWVARIYPTNGYTRYADSVKGRFTISADTSKN |
| | VL_{(CD3-MABdeimm}_5) - | | TAYLQMNSLRAEDTAVYYCSRWGGDGFYAMDYWGQG |
| | Linker_{(20 aaCLλ)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSVVTV |
| | CH3_{(knob)} | | PSSSLGTQTYICNVNHKPSNTKVDKRVEPKSCAQPAAPA |
| | | | PDAHEAPAPAQGSQSVLTQPPSASGTPGQRVTISCSGS |
| | | | SSNIGSNYVYWYQQLPGTAPKLLIYRNNQRPSGVPDRFS |
| | | | GSKSGTSASLAISGLRSEDEADYYCAGWSRELHGAVFG |
| | | | GGTKLTVLGQPKAAPSVTLFPPSSEELQANDKTHTCPPC |
| | | | PAPEAEGAPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPSSIEKTISKAKGQPRE |
| | | | PQVYTLPPCREEMTKNQVSLWCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | VH_{(Trast.)} - CH1 | 75 | QVQLVESGGGLVQPGGSLRLSCAASGFNIKDTYIHWVR |
| | Linker_{(20 aaPAPDA)} - | | QAPGKGLEWVARIYPTNGYTRYADSVKGRFTISADTSKN |
| | VH_{(CD3-MABdeimm}_5) - | | TAYLQMNSLRAEDTAVYYCSRWGGDGFYAMDYWGQG |
| | Linker_{(20 aaCH1)} - | | TLVTVSSASTKGPSVFPLAPSSKSTSGGTAALGCLVKDY |
| | CH2_{(AEASS)} - | | FPEPVTVSWNSGALTSGVHTFPAVLQSSGLYSLSSVVTV |
| | CH3₍ₕₒₗₑ₎ | | PSSSLGTQTYICNVNHKPSNTKVDKRVEPKSCAQPAAPA |
| | | | PDAHEAPAPAQGSEVQLVESGGGLVQPGGSLRLSCAAS |
| | | | GFSFGSHYMSWVRQAPGKGLEWVANINQIGYSSYYGES |
| | | | VKGRFTISRDNAKNSLYLQMNSLRAEDTAVYYCARGYSD |
| | | | EFAKRSGLDVWGQGTLVTVSSASTKGPSVFPLAPSSKS |
| | | | TSGDKTHTCPPCPAPEAEGAPSVFLFPPKPKDTLMISRT |
| | | | PEVTCWVDVSHEDPEVKFNWYVDGVEVHNAKTKPREE |
| | | | QYNSTYRVVSVLTVLHQDWLNGKEYKCKVSNKALPSSIE |
| | | | KTISKAKGQPREPQVCTLPPSREEMTKNQVSLSCAVKGF |
| | | | YPSDIAVEWESNGQPENNYKTTPPVLDSDGSFFLVSKLT |
| | | | VDKSRWQQGNVFSCSVMHEALHNHYTQKSLSLSPGK |
| | VL_{(Trast.)} - CL | 51 | DIQMTQSPSSLSASVGDRVTITCRASQDVNTAVAWYQQ |
| | | | KPGKAPKLLIYSASFLYSGVPSRFSGSRSGTDFTLTISSL |
| | | | QPEDFATYYCQQHYTTPPTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYACE |
| | | | VTHQGLSSPVTKSFNRGEC |

### EXAMPLES

### Example 1: Identification of Affinity Optimized CD3 Specific Human Antibodies

Generation of the grandparent human cynomolgus CD3 cross-reactive fully human antibody CD3-MAB_{GP} (comprising the VH of SEQ ID NO: 25 and the VL of SEQ ID NO: 26) is described in PCT/EP2021/076052. In brief, for identification of CD3-MAB_{GP}, the MorphoSys Ylanthia® library was used to select Fab fragments against human and cynomolgus CD3. The MorphoSys Ylanthia® library (Tiller et al. mAbs 5:3, 1-26; May/June (2013) and U.S. Pat. No. 8,728,981) is a commercially available phagemid library and employs the CysDisplay® technology for displaying the Fab on the phage surface (Lohning et al., WO2001/05950). To further increase affinity, species cross-reactivity and biological activity, the LCDR3 and HCDR1/HCDR2 regions of CD3-MAB_{GP} were optimized in parallel using diversified Ylanthia® maturation modules that were generated previously with the Slonomics® technology (van den Brulle et al. 2008). For the selection of affinity improved derivatives of CD3-MAB_{GP}, phage derived from maturation libraries were subjected to three rounds of maturation pannings. Panning stringency was increased by lowering the CD3 antigen concentration or cell count of CD3 expressing cells in each panning round (Low et al. 1996). In addition to antigen reduction, off-rate selections were performed (Hawkins et al. 1992) using excesses of unbiotinylated CD3epsilon antigens as competitor to further increase selection stringency. All strategies were combined with prolonged washing steps.

### Example 1.1: SET Affinity Screening after Affinity Maturation

For generation of Fab containing crude bacterial lysates (BEL extracts) used for primary affinity screenings, microtiter plates pre-filled with growth medium (2×YT containing chloramphenicol, IPTG and low glucose) were inoculated using Fab containing bacterial glycerol stocks. Plates were incubated at 37° C. for bacterial outgrowth and shaken overnight at 22° C. for Fab expression. The next day expression cultures were lysed by addition of BEL buffer containing borate buffer, EDTA and lysozyme. Depending on the selected plate format and application, volumes were adjusted.

SET (Solution Equilibrium Titration) screening (Della Ducata et al. 2015) was in principle performed as described by Haenel and coworkers (Haenel et al. 2005). A constant amount of diluted Fab containing BEL extract was equilibrated over night with different concentrations of CD3epsilon antigen (hCD3e(22-118)_FLAG_chLys_avi (SEQ ID NO: 43). The mixture was then transferred to MSD plates, which were previously coated with antigen, and after incubation and washing, a suitable MSD-Sulfo-tag labeled detection antibody was added. Subsequently, the concentration of unbound Fab was quantified via ECL detection using the Sector Imager 6000 (Meso Scale Discovery|Gaithersburg|MD|USA). Results were processed using XLfit (IDBS) software, applying the corresponding fit model as described above to estimate affinities and thus identify clones most improved by maturation.

Approx. 1000 clones obtained from the 3^{rd} panning round output were screened for affinity of which 189 clones with SET K_{D} estimates of <40 nM on recombinant human CD3epsilon antigen were subjected for VH or VL sequencing, as required. Sequencing resulted in the identification of 101 sequence unique HCDR1+2 affinity matured derivatives but only 15 sequence unique LCDR3 affinity matured derivatives of CD3-MAB_{GP}.

### Example 1.2: Conversion into and Production of a Bispecific 2+1 Fab₂-Fc-scFv Antibody Format

In order to determine the suitability of sequence unique affinity optimized CD3 specific antibodies of Example 1.1 to be used in bispecific antibody formats, the VH and VL of CD3-MAB_{GP}, of 48× sequence unique HCDR1-3 and 3× sequence unique LCDR3 derivatives with SET K_{D} estimates of <10 nM on human CD3epsilon were converted into a bispecific 2+1 Fab₂-Fc-scFv antibody format as shown in FIG. 1A. This bispecific antibody format is built from an aglycosylated human IgG1 backbone and encompasses one extra scFv fragment, with the N-terminus of the scFv VL domain fused via a peptide linker to the C-terminus of one IgG heavy chain. To facilitate heterodimerization of the two different heavy chains, knob-into-hole mutations were introduced in both CH3-Fc domains. In the present examples, both Fabs arms of the IgG backbone bind to tumor target HER2 while the extra scFv comprises the variable domains of the CD3 specific antibodies of the present disclosure. For HER2 binding, nucleotide sequences encoding the VH and VL domains from “Trastuzumab” (HERCEPTIN®) as described by Baselga et al. 1998, Cancer Res 58(13): 2825-2831) were used. Trastuzumab and its method of preparation are described in U.S. Pat. No. 5,821,337. A summary of the polypeptide sequences forming the exemplary bispecific antibody Bissig_08_#1 carrying the affinity improved VL domain of CD3-MAB_{opt_VL} (SEQ ID NO: 25 and SEQ ID NO: 20) or the exemplary bispecific antibody Bissig_08_#2 carrying the affinity improved VH domain of CD3-MAB_{opt_VH}#1 (SEQ ID NO: 14 and SEQ ID NO: 26) made in accordance with Example 1.2 are set forth in Table 10.

All nucleic acid sequences or desired gene segments either were generated by PCR using appropriate templates or were gene synthesized as linear DNA fragments with appropriate flanking regions (e.g. suitable restriction enzyme recognition sites, linker sequences) in-house or by an external provider. The nucleic acid sequences or gene segments flanked by singular restriction endonuclease cleavage sites were cloned into respective mammalian expression vectors using standard molecular biology methods. When intended for use in mammalian expression vectors, all constructs were designed with a 5′-end DNA sequence coding for a leader peptide which targets proteins for secretion in eukaryotic cells. The DNA sequence of the subcloned gene fragments was confirmed by DNA by double strand sequencing. Eukaryotic HEK293-6E cells were transfected with mammalian expression vector DNA encoding all components of the heavy and light chains of the bispecific antibody, resulting in the 2:1:1 heterodimeric bispecific antibody. Cell culture supernatants were harvested 7 days post transfection and subjected to Protein A affinity chromatography (MabSelect SURE|GE Healthcare) using a liquid handling station. Samples remained in neutralized elution buffer (NaPS: 137 mM NaPhosphate, 81 mM NaCl, pH 7). Samples were sterile filtered (0.2 μm pore size). Protein concentrations were determined by UV-spectrophotometry and purities of antibodies were analyzed under denaturing, reducing conditions using CE-SDS (LabChip GXII|Perkin Elmer|USA). HP-SEC was performed to analyze bispecific antibody preparations in native state.

In sum, 41/51 bispecific antibody could be produced in acceptable quality and yield with a monomer content of >85% as determined by analytical size exclusion chromatography. Table 16 provides an overview of the monomer content of 7 produced bispecific antibody preparations comprising the variable domains of either the grandparent CD3 antibody CD3-MAB_{GP} or of 6 preferred affinity optimized CD3 antibodies as described above, including CD3-MABopt_VL and CD3-MABopt_VH #1.

### Functional Characterization of Affinity Optimized CD3 Specific Antibodies in the Bispecific 2+1 Fab₂-Fc-scFv Antibody Format

All produced bispecific antibodies of Example 1.2 were tested in in vitro assays comprising:
Affinity determination by surface plasmon resonance (SPR)
Binding to human T cells and Jurkat cells with endogenous expression of CD3 and J.RT-T3.5 cells without CD3 expression
Functional NFAT Reporter Gene Assay using the cancer cell line SKBR3.
Functional cytotoxicity assay on SKBR3 cells.

### Example 1.3: K_{D} Determination Via Antibody Capture Setup

Kinetic characterization of the interaction between human CD3epsilon and the 41 produced bispecific Fab₂-Fc-scFv antibodies of Example 1.2 was carried out in antibody capture format, with the antigen being applied as analyte in solution. High-capacity capture surfaces were prepared by loading biotinylated MabSelect SuRe ligand (non-biotinylated ligand: GE Healthcare, 28-4018-60) onto several streptavidin sensors (fortébio, part 18-5021). Each cycle of the kinetic experiment consisted of capture steps (of one ligand on several sensors used in parallel), followed by an analyte binding step (association phase, different analyte concentrations and assay buffer, i.e. antigen concentration 0 for blank subtraction). After binding, the dissociation of bound antigen was monitored (sensors exposed to assay buffer). At the end of each cycle, bound ligand and/or ligand-antigen complex was removed from the sensor surfaces by 2 consecutive regeneration steps a 20 s with 10 mM Glycine/HCl pH1.5 (GE Healthcare, BR 100354), while maintaining the integrity of the capture surface. Signals recorded on the sensor with captured ligand, but exposed to assay buffer instead of antigen during binding were subtracted from the sensorgrams with non-zero antigen concentrations to correct e.g. potential dissociation of captured ligand. Association was recorded for 300 s and dissociation for 300 s at an orbital shaking speed of 1000 rpm. DPBS (GIBCO, no Ca²⁺ no Mg²⁺; Thermo Fisher Cat. No. 14190) supplemented with 0.05% (v/v) Polysorbate 20 (Merck, 8.22184.0500) and 0.1% (w/v) bovine serum albumin (Sigma, A7906) was used as assay buffer. Capture levels of ligands were adjusted to approx. 2 nm to achieve saturation levels Rmax of approx. 0.2 nm by the human CD3e analyte. Seven different analyte concentrations were used for analysis during kinetic experiments (hCD3e(22-118)_F-chLys_avi (SEQ ID NO: 43); applied molarities 15.6-1000 nM, in a 2-fold serial dilution series). Sensorgrams were evaluated with Data Analysis Software v 10 (Octet/fortébio). All sensorgrams were fitted to a 1:1 binding model to determine kₒₙ and k_{off} rate constants, which were used to calculate the K_{D} value. For kinetic profiles deviating from the expected 1:1 binding, the sensorgrams were evaluated using a best approximation to the monovalent kinetics, and results marked with comment “heterogeneous binding”. These results are considered less precise than kinetic profiles completely following the expected monovalent binding kinetics, but are assumed to be good approximations for K_{D}.

Overall, determined monovalent affinities for all 41 tested affinity optimized CD3 specific antibodies on human CD3e antigen were in the range of 39 nM to 120 nM, whereas the grandparent antibody CD3-MAB_{GP} revealed an K_{D} of 240 nM when tested in the same bispecific antibody format. Table 16 summarizes the results of the kinetic studies determined for CD3-MAB_{GP} and 6 preferred affinity optimized CD3 antibodies according to the present disclosure, including those for CD3-MABopt_VL (BissIg_08_#1) and CD3-MABopt_VH #1 (BissIg_08_#2).

### Example 1.4: Cell Binding

41 produced bispecific Fab₂-Fc-scFv antibodies of Example 1.2 comprising affinity improved CD3 specific antibodies were tested for their ability to bind to CD3 positive human T cells and to the CD3 negative cell line J.RT3-T3.5.

For isolation and purification of human T cells, human whole blood from healthy donors was collected in Li-Heparin containing S-Monovette containers (Sarstedt). Blood was transferred to 50 ml conical tubes and mixed with an equal volume of PBS containing 2% fetal bovine serum (Sigma, #F7524) and 2 mM EDTA. Diluted blood was transferred to SepMate-50 tubes (StemCell Technologies, #86450) containing 15 ml Biocoll solution (Biochrom, #L6115) and centrifuged for 10 min at 1200×g. Supernatant was transferred into a 50 ml conical tube, diluted to 45 ml with PBS and centrifuged for 8 min at 300×g. Supernatant was discarded, cell pellet resuspended in 1 ml PBS and cells counted using a Neubauer chamber. T cells were isolated and purified using the EasySep™ Human T Cell isolation kit (StemCell Technologies) according to the providers instructions. Purity assessment of CD3+ T cells was done by flow cytometry with an anti-human CD3 PE conjugated antibody (Biolegend #12-0037-42). Jurkat and J.RT3-T3.5. cells were resuspended and counted in Superblock (ThermoScientific, #37515) and blocked for 1 h on ice. Blocked cells were resuspended with bispecific antibodies serially diluted in Superblock (starting final concentration: 500 nM-0.69 nM/0.23 nM; 1:3 dilution series) and incubated for 1 h on ice. Cells were washed 2 times in D-PBS (Gibco) containing 3% fetal bovine serum (Sigma, #F7524). Bound bispecific antibodies were detected using AlexaFluor 647 labeled goat anti-Human IgG, F(ab′)2 Fragment specific (Jackson Immuno Research Cat #109-606-097). Antibody staining was measured using FACS Array (Beckton Dickinson) or IntelliCyt iQue flow cytometer and analyzed in FlowJo or ForeCyt (IntelliCyt) softwares, respectively. EC₅₀ values were calculated using 4-parameter non-linear regression analysis in Prism software (GraphPad Software Inc.).

Table 16 summarizes the results of the cell binding studies determined for the bispecific antibodies according Example 1.2 comprising the variable domains of CD3-MAB_{GP} or 6 preferred affinity optimized CD3 antibodies according to the present disclosure, including those for CD3-MABopt_VL (BissIg_08_#1) and CD3-MABopt_VH #1 (BissIg_08_#2). Cell binding is shown as signal over background ratios for an selected antibody concentration of 167 nM. The affinity improved CD3 specific antibodies exhibited significant stronger signal intensities on human T cells when compared to grandparent antibody CD3-MAB_{GP}. Furthermore, no or only very weak binding to the CD3 negative cell line J.RT3-T3.5. was observable (data no shown).

### Example 1.5 Jurkat NFAT Reporter Gene Cell Assay

For the evaluation of the functional activity of the bispecific Fab₂-Fc-scFv antibodies according Example 1.2, Jurkat cells (ATCC #TIB-152) transiently transfected with an NFAT reporter gene construct were used as surrogate effector cells. As target cells, the HER2 positive human adenocarcinoma SKBR-3 (ATCC® HTB-30™) cell line was used. The following growth media were used for maintenance of the cell lines: (a) Jurkat: RPMI-1640+L-Glutamine (Thermo Fisher, #21875-034) supplemented with 10% FCS (Sigma, #F7524); (b) SKBR-3: McCoys 5a (Gibco, #26600), supplemented with 10% FCS (Sigma #F7524) SKBR-3 cells were diluted in growth medium to a density of 4E+05 cells/ml. 100 μl cell suspension corresponding to 40,000 cells were seeded in each well of a tissue culture treated 96 well plate (Corning, #3917) and incubated overnight in a humidified incubator at 37° C. and 5% CO₂. Jurkat cells were resuspended in growth medium to a concentration of 2.5E+05 cells/ml. Transfection components pGL4.30[luc2P/NFAT-RE/Hygro] reporter gene vector (Promega #9PIE848), OptiMEM-I medium (Life Technologies, #31985-047) and TransIT-LT1 transfection reagent (Mirus, #MIR2304) were incubated for 15 min at RT, then added to the Jurkat cell suspension and incubated for 17 h in a humidified incubator at 37° C. and 5% CO₂. Jurkat cells were harvested and resuspended in growth medium at a concentration of 1.2E+06/ml. Medium was removed from coated target cells and replaced by 50 μl Jurkat cell suspension corresponding to 60,000 cells per well. Bispecific antibodies were serially diluted in Jurkat growth medium. 50 μl antibody dilution was added to each well resulting in a final concentration range of 10 nM to 0.01 nM (4 step dilution). Assay plates were incubated for 5 h in a humidified incubator at 37° C. and 5% CO₂. Bright-Glo™ Reagent (Promega, #E2620) was reconstituted according to manufacturer's instructions. Assay plates and reagent were equilibrated at room temperature. 100 μl of the Bright-Glo™ reagent was added to each well of the assay plate and mixed. Luminescence was measured using an InfiniteM1000 Pro plate reader (Tecan).

Table 16 summarizes the results of the reporter gene assay determined for bispecific antibodies comprising the variable domains of CD3-MAB_{GP} or 6 preferred affinity optimized CD3 specific antibodies according to the present disclosure, including those for CD3-MABopt_VL (BissIg_08_#1) and CD3-MABopt_VH #1 (BissIg_08_#2). Results are shown as signal over background ratios for an bispecific antibody concentration of 1 nM. Bispecific antibodies comprising affinity optimized CD3 specific antibodies exhibited significant stronger activation of the reporter gene system in Jurkat cells when compared to the bispecific antibody comprising the variable domains of grandparent CD3 specific antibody CD3-MAB_{GP}, which hardly showed any activity.

### Example 1.6: Cytotoxicity Assays with Bispecific Antibodies

41 bispecific Fab₂-Fc-scFv antibodies comprising affinity optimized CD3 specific antibodies according Example 1.2 were tested for their ability to mediate T cell dependent killing of HER2 expressing SKBR3 cells or HER2 negative MDA-MB468 cells. PBMC were prepared as described in Example 1.4. 5,000 SKBR3 or MDA-MB468 cells were suspended in culture medium (SKBR3: McCoy's 5A Medium (Gibco, #26600), 10% FCS (Sigma, #F7524); MDA-MB468: DMEM (Gibco, #10938), GlutaMax (Gibco, #35050), 10% FCS, 1× Sodium Pyruvate (Gibco, #11360-039)), seeded in black 96 well assay plates (Corning, #3340) and incubated over night at 37° C. and 5% CO₂. CellToxGreen dye (Promega, #G8731), serially diluted bispecific antibody constructs (final concentration: 5 nM-100 pM) and 100,000 purified PBMCs, all diluted in assay medium comprising RPMI 1640 w/o Phenol red (Gibco, #32404-014), GlutaMax and 10% fetal bovine serum, were added to the cells and incubated for 48 h at 37° C. and 5% CO₂. Cytotoxic activity was assessed after 72h by measuring incorporated CellToxGreen fluorescence at 485 nm excitation and 535 nm emission using a Tecan Infinite F500 device. EC₅₀ values were calculated using 4-parameter non-linear regression analysis in Prism software (GraphPad Software Inc.).

Overall, only 24 tested bispecific antibodies comprising affinity optimized CD3 specific variable domains of Example 1.2 mediated T cell killing of SKBR3 cells. No killing was observable for the bispecific antibody comprising the variable domains of grantparent antibody CD3-MAB_{GP} in line with the findings of the RGA assay of Example 1.5. Table 16 summarizes the results (IC₅₀ conc.) of the T cell redirected killing of SKBR3 cells determined for bispecific antibodies comprising the variable domains of CD3-MAB_{GP} or of 6 preferred affinity optimized CD3 antibodies according to the present disclosure, including those for CD3-MABopt_VL (BissIg_08_#1) and CD3-MABopt_VH #1 (BissIg_08_#2).

### Example 1.7: ELISA Binding

Bispecific Fab₂-Fc-scFv antibodies according Example 1.2 were tested for their ability to bind to recombinant human and cynomolgus CD3epsilon antigen in ELISA. 5 nM of recombinant human CD3e (22-49)-Fc2 (K105-K330) (SEQ ID NO: 41) or 1 nM of recombinant cynomolgus CD3e (22-49)-Fc2(K105-K330) (SEQ ID NO: 42) were coated on Maxisorp plates (Nunc, #460518). Coated plates were blocked with 5% skim milk in PBS. Antibodies were serially diluted in PBS containing 0.5% skim milk and 0.5% Tween-20. Bound antibodies were detected using an alkaline phosphatase-conjugated detection antibody directed against human F(ab′)2 fragment (Jackson Immuno Research, #109-055-097). EC₅₀ values were calculated using 4-parameter non-linear regression analysis in Prism software (GraphPad Software Inc.)

Table 15 summarizes ELISA EC₅₀ values determined for the bispecific antibodies comprising the variable domains of grandparent antibody CD3-MAB_{GP} and for 6 preferred affinity optimized CD3 antibodies according to the present disclosure, including those for CD3-MABopt_VL (BissIg_08_#1) and CD3-MABopt_VH #1 (BissIg_08_#2). The results reveal that the affinity optimized CD3 specific antibodies exhibited 10 to 20 fold improved binding to human and cynomolgus monkey CD3epsilon antigen when compared to the grandparent antibody CD3-MAB_{GP}.

TABLE 15
ELISA binding of affinity improved CD3 specific antibodies of the present
disclosure to recombinant human or cynomolgus CD3epsilon antigens when tested
in the bispecific antibody format Fab₂-Fc-scFv of Example 1.

| Bispecific antibody | | anti-CD3 antibody | | | | |
|---|---|---|---|---|---|---|
| | SEQ ID | | SEQ ID | SEQ ID | EC₅₀ [nM] | |
| | Nos | | NO: | NO: | hCD3e(22-49)_{—} | cyCD3e(22-49)_{—} |
| | (Table 10) | | VH | VL | Fc2(K105-K330) | Fc2(K105-K330) |
| Bisslg_08_#GP | n.a | CD3-MAB_{GP} | 25 | 26 | 248.0 | 369 |
| Bisslg_08_#3 | n.a | CD3-MABopt_VH#2 | n.a. | n.a. | 11.8 | 15.6 |
| Bisslg_08_#4 | n.a | CD3-MABopt_VH#3 | n.a. | n.a. | 17.0 | 30.9 |
| Bisslg_08_#5 | n.a | CD3-MABopt_VH#4 | n.a. | n.a. | 9.9 | 11.0 |
| Bisslg_08_#6 | n.a | CD3-MABopt_VH#5 | n.a. | n.a. | 20.3 | 30.9 |
| Bisslg_08_#2 | 52, 50, 51 | CD3-MABopt_VH#1 | 14 | 26 | 11.1 | 15.0 |
| Bisslg_08_#1 | 49, 50, 51 | CD3-MABopt_VL | 25 | 20 | 26.1 | 29.5 |

n.a.: not applicable

### Summary Functional Characterization of Affinity Improved Antibody

Affinity maturation of grandparent antibody CD3-MAB_{GP} resulted in the identification of 5 affinity optimized HCDR1-2 variants and 1 affinity optimized LCDR3 variant of CD3-MAB_{GP} with preferred characteristics and suited to be used in bispecific antibody formats Table 16 provides an overview of the favorable functional and biophysical properties of these antibodies compared to the grandparent antibody when tested in the bispecific antibody format of Example 1.

TABLE 16
Summary functional functional and biophysical properties of affinity optimized
CD3 specific antibodies according to the present disclosure.

| | | | | | | | | T cell | RGA @ | |
|---|---|---|---|---|---|---|---|---|---|---|
| | | anti-CD3 antibody | | | | | | binding @ | antibody | |
| Bispecific antibody | | | SEQ | SEQ | | SEC | K_{D} | antibody | conc. of | SKBR3 |
| | SEQ ID | | ID | ID | | monomer | [nM] | conc. of | 1 nM | killing |
| | Nos | | NO: | NO: | Optimized | content | human | 167 nM | [S/BG] | IC₅₀ |
| | (Table 10) | | VH | VL | CDR | [%] | CD3e | [S/BG] | SKBR3 | [pM] |
| Bisslg_08_#GP | n.a | CD3-MAB_{GP} | 25 | 26 | n.a. | 85.7 | 240 | 1.4 | 1 | No |
| | | | | | | | | | | activity |
| Bisslg_08_#3 | n.a | CD3-MABopt_VH#2 | n.a. | n.a. | HCDR1 + 2 | 87.1 | 83 | 13.9 | 917 | 26 |
| Bisslg_08_#4 | n.a | CD3-MABopt_VH#3 | n.a. | n.a. | HCDR1 + 2 | 86.0 | 94 | 8.2 | 209 | 39 |
| Bisslg_08_#5 | n.a | CD3-MABopt_VH#4 | n.a. | n.a. | HCDR1 + 2 | 86.5 | 39 | 14.4 | 693 | 82 |
| Bisslg_08_#6 | n.a | CD3-MABopt_VH#5 | n.a. | n.a. | HCDR1 + 2 | 85.3 | 93 | 11.6 | 205 | 59 |
| Bisslg_08_#2 | 52, 50, 51 | CD3-MABopt_VH#1 | 14 | 26 | HCDR1 + 2 | 86.1 | 55 | 15.8 | 306 | 46 |
| Bisslg_08_#1 | 49, 50, 51 | CD3-MABopt_VL | 25 | 20 | LCDR3 | 85.4 | 62 | 20.0 | 367 | 40 |

n.a.: not applicable

### Example 2: Further Optimizations of Affinity Improved CD3 Specific Antibodies—Cross-Cloning of Affinity Optimized Variable Domains and Conversion into a Further Bispecific Antibody Format

To further improve the functional characteristics of the previously identified affinity optimized CD3 specific antibodies, 5 cross-clones were generated by combining the VH of CD3-MABopt_VH #1 (SEQ ID NO: 14), CD3-MABopt_VH #2, CD3-MABopt_VH #3, CD3-MABopt_VH #4, and CD3-MABopt_VH #5 with the VL of CD3-MABopt_VL (SEQ ID NO: 20).

Subsequently, affinity improved antibodies CD3-MABopt_VH #1, CD3-MABopt_VH #2, CD3-MABopt_VH #3, CD3-MABopt_VH #4, CD3-MABopt_VH #5 and CD3-MABopt_VL as well as the 5 generated cross-clones from these were converted into a bispecific 1+1 antibody format as shown in FIG. 1B. The bispecific antibody format of Example 2 has the typical Y-shape of a conventional IgG molecule with one Fab arm binds to a tumor target (HER2) and the other Fab arm binds to CD3. For HER2 binding, the VH and VL domain of “Trastuzumab” (HERCEPTIN©) (SEQ ID NO: 28 and SEQ ID NO 29, respectively) as described by Baselga et al. 1998, Cancer Res 58(13): 2825-2831) were used. Trastuzumab and its method of preparation are described in U.S. Pat. No. 5,821,337.

The bispecific 1+1 antibodies were generated in vitro by 2-MEA-induced Fab-arm exchange as described in WO2011147986, WO2011131746 and WO2013060867 and Labrijn et al. (Labrijn et al., PNAS 2013, 110: 5145-50; Gramer et al., MAbs 2013, 5: 962-973). To enable the production of bispecific antibodies by this method, two source IgG1 molecules carrying a mutation in the CH3 domain were generated: in one source IgG1 antibody the F405L mutation (i.e. the CD3 specific antibody), in the other source IgG1 antibody the K409R mutation (e.g. the anti-HER2 antibody). In addition to these mutations, the source IgG1 molecules included substitutions that result in a Fc region that is unable to interact with IgG Fc receptors (Fc gamma receptors) and complement: L234A, L235E, G237A, A330S and P331S (“AEASS”).

A summary of the polypeptide sequences forming two exemplary produced source IgG1 molecules with specificity for HER2 (IgG #1: SEQ ID NOs: 53 and 51) or CD3 (IgGopt_cc: (SEQ ID NOs: 54 and 55), respectively and made in accordance with Example 2 are set forth in Table 11. The CD3 specific IgG comprises the VH and VL of cross-clone CD3-MAB_{opt-cc} (SEQ ID NO: 14 and SEQ ID NO: 20, respectively). A summary of the polypeptide sequences forming the resulting (after Fab arm exchange) bispecific 1+1 antibody BissIg_18_opt_cc #(SEQ ID NOs: 53, 51, 54, 55) is set forth in Table 12.

For regular production of a source IgG1 molecule, Eukaryotic HEK293-6E cells were transfected with mammalian expression vector DNA encoding the heavy and light chain of the source IgG molecules. Cell culture supernatants were harvested on day 3 or 6 post transfection and subjected to standard Protein A affinity chromatography (MabSelect SURE|GE Healthcare). Buffer exchange was performed to 1× Dulbcecco's PBS (pH 7.2|Invitrogen) and samples were sterile filtered (0.2 μm pore size). Protein concentrations were determined by UV-spectrophotometry and purities of IgG were analyzed under denaturing, reducing and non-reducing conditions using CE-SDS (LabChip GXII|Perkin Elmer|USA). HP-SEC was performed to analyze IgG preparations in native state.

To generate the bispecific 1+1 antibodies via Fab arm exchange, the two produced source IgGs were mixed in equal mass amounts in PBS buffer (Phosphate Buffered Saline; 8.7 mM HPO₄⁻², 1.8 mM H₂PO⁴⁻, 163.9 mM Na+, 140.3 mM Cl—, pH 7.4). 2-mercaptoethylamine-HCl (2-MEA) was added to a final concentration of 75 mM and the reaction mixture was incubated at room temperature for 5 h. The 2-MEA was removed by buffer exchange into PBS using PD-10 columns to allow re-oxidation of the interchain disulfide bonds and formation of intact bispecific antibodies. Protein concentrations were determined by UV-spectrophotometry and purities of bispecific IgG preparations were analyzed under denaturing, reducing and non-reducing conditions using CE-SDS (LabChip GXII|Perkin Elmer|USA). HP-SEC was performed to analyze bispecific IgG preparations in native state.

Table 17-1 summarizes quality control of mammalian produced source IgG1 molecules for affinity improved CD3 specific antibodies CD3-MABopt_VH #1, CD3-MABopt_VH #2, CD3-MABopt_VH #3, CD3-MABopt_VH #4, CD3-MABopt_VH #5, and CD3-MABopt_VL according Example 1 before controlled Fab arm exchange (FAE). Table 17-2 summarizes quality control of mammalian produced source IgG1 molecules of corresponding cross-cloned CD3 specific antibodies CD3-MAB_{opt_cc}, CD3-MAB_{opt_cc #2}, CD3-MAB_{opt_cc #3}, CD3-MAB_{opt_cc #4} and CD3-MAB_{opt_cc #5} according Example 2 before controlled Fab arm exchange (FAE).

Overall, IgGs comprising cross-cloned CD3 specific antibody variable domains revealed a lower monomer content of IgG preparation when compared to the IgGs comprising the solely affinity improved CD3 specific antibody variable domains.

TABLE 17-1
Quality control of produced source IgGs comprising affinity
optimized CD3 specific antibodies before Fab arm exchange

| | | | | monomer |
|---|---|---|---|---|
| | Affinity optimized | Seq ID | Seq ID | content [%] |
| IgG | anti-CD3 antibody | NO: VH | NO: VL | IgG |
| IgG#opt_VH#2 | CD3-MABopt_VH#2 | n.a. | 20 | 95.1 |
| IgG#opt_VH#3 | CD3-MABopt_VH#3 | n.a. | 20 | 84.8/94.3 |
| IgG#opt_VH#4 | CD3-MABopt_VH#4 | n.a. | 20 | 90.6 |
| IgG#opt_VH#5 | CD3-MABopt_VH#5 | n.a. | 20 | 90.6 |
| IgG#opt_VH#1 | CD3-MABopt_VH#1 | 14 | 26 | 96.1 |
| IgG#opt_VL | CD3-MABopt_VL | 25 | 20 | 90.8 |

n.a.: not applicable

TABLE 17-2
Quality control of produced source IgGs comprising
CD3 specific cross-clones before Fab arm exchange

| IgG | | Cross-Cloned anti-CD3 antibody | | monomer |
|---|---|---|---|---|
| | Seq ID NOs | | Originating a-CD3 | content [%] |
| ID | (HC, LC) | ID | variable domains | IgG |
| IgG#opt_cc#2 | n.a. | CD3-MABₒₚₜ_{_{—}}_{cc#2} | CD3-MABopt_VH#2 | 85.9 |
| | | | CD3-MABopt_VL | |
| IgGopt_cc#3 | n.a. | CD3-MABₒₚₜ_{_{—}}_{cc#3} | CD3-MABopt_VH#3 | 78/84.5 |
| | | | CD3-MABopt_VL | |
| IgGopt_cc#4 | n.a. | CD3-MABₒₚₜ_{_{—}}_{cc#4} | CD3-MABopt_VH#4 | 80.8/90.2 |
| | | | CD3-MABopt_VL | |
| IgGopt_cc#5 | n.a. | CD3-MABₒₚₜ_{_{—}}_{cc#5} | CD3-MABopt_VH#5 | 90.9 |
| | | | CD3-MABopt_VL | |
| IgGopt_cc# | 54, 55 | CD3-MABₒₚₜ_{_{—}}_{cc} | CD3-MABopt_VH#1 | 82.8/94.4/95.7 |
| | | | CD3-MABopt_VL | |

Table 18 summarizes quality control of bispecific IgG preparation after controlled Fab arm exchange (FAE) comprising cross-cloned CD3 specific binding domains. Overall, the bispecific antibody preparations revealed a high monomer content when compared to the corresponding source monospecific IgG preparations.

TABLE 18
Quality control of generated bispecific antibodies comprising cross-
cloned CD3 specific binding domains after Fab arm exchange.

| Bispecific antibody | | Cross-Cloned anti-CD3 antibody | | monomer |
|---|---|---|---|---|
| | Seq ID | | Originating a-CD3 | content [%] |
| ID | NOs | ID | variable domains | IgG |
| BissIg_18_opt_cc#2 | n.a. | CD3-MABₒₚₜ_{_{—}}_{cc#2} | CD3-MABopt_VH#2 | 89.8 |
| | | | CD3-MABopt_VL | |
| BissIg_18_opt_cc#3 | n.a. | CD3-MABₒₚₜ_{_{—}}_{cc#3} | CD3-MABopt_VH#3 | 87.3 |
| | | | CD3-MABopt_VL | |
| BissIg_18_opt_cc#4 | n.a. | CD3-MABₒₚₜ_{_{—}}_{cc#4} | CD3-MABopt_VH#4 | 87.1 |
| | | | CD3-MABopt_VL | |
| BissIg_18_opt_cc#5 | n.a. | CD3-MABₒₚₜ_{_{—}}_{cc#5} | CD3-MABopt_VH#5 | 90.4 |
| | | | CD3-MABopt_VL | |
| BissIg_18_opt_cc# | 51, 53, | CD3-MABₒₚₜ_{_{—}}_{cc} | CD3-MABopt_VH#1 | 88.3 |
| | 54, 55 | | CD3-MABopt_VL | |

### Example 2.1: K_{D} Determination Via Antibody Capture Setup

Affinity determination by determining kinetic rate constants was performed on an Octet HTX (FortéBIO, Sartorius AG) instrument. Bispecific antibody preparations of Example 2 diluted in assay buffer (DPBS (GIBCO, no Ca²⁺, no Mg²⁺; Thermo Fisher Cat. No. 14190) supplemented with 0.05% (v/v) Polysorbate 20 (Merck, 8.22184.0500) and 0.1% (w/v) bovine serum albumin (Sigma, A7906) were captured onto IgG-specific BLI sensors with a loading level of approx. 2 nm. For analysis, human CD3 epsilon antigen hCD3e(22-118)_F-chLys_avi-biotin (SEQ ID NO: 43) was diluted with assay buffer to concentrations ranging from 7.8 nM to 500 nM (serial 1:2 dilutions). A blank sample with assay buffer was included for referencing, i.e. correcting for dissociation of captured antibody. The association phase was recorded for 180 s, followed by a dissociation phase of 300 s. After each cycle, the biosensors were regenerated two times with 10 mM Glycine HCl pH 1.7 to remove bound ligand/antibody complex, while maintaining the integrity of the capture surface. Between regeneration steps, biosensors were washed with assay buffer for 20 s. Sensorgrams were evaluated with Data Analysis Software v 10 (Octet/fortébio). All sensorgrams were fitted to a 1:1 binding model to determine kₒₙ and k_{off} rate constants, which were used to calculate the K_{D} value. For kinetic profiles deviating from the expected 1:1 binding, the sensorgrams were evaluated using a best approximation to the monovalent kinetics, and results marked with comment “heterogeneous binding”. These results are considered less precise than kinetic profiles completely following the expected monovalent binding kinetics, but are assumed to be good approximations for K_{D}.

Table 19 summarizes K_{D} values of bispecific antibodies comprising affinity optimized or cross-cloned CD3 specific binding domains according to the present disclosure. The optimized or cross-cloned CD3 specific antibodies recognized recombinant human CD3epsilon antigen with K_{D} values in the low single digit nanomolar range except for the HCDR1-2 matured antibody CD3-MABopt_VH #1 (SEQ ID NO: 14 and SEQ ID NO: 26) and the LCDR3 matured antibody CD3-MABopt_VL (SEQ ID NO: 25 and SEQ ID NO: 20). These two antibodies revealed heterogenous binding to CD3e in the low double digit nanomolar rage. Surprisingly it was found, that the generated cross-clone CD3-MABopt_cc (SEQ ID NO 14 and SEQ ID NO: 20) which is build from the VH of CD3-MABopt_VH #1 and the VL of CD3-MABopt_VL showed a ˜20 to 30 fold improved binding affinity to CD3e when compared to its originating antibodies.

TABLE 19
K_{D} values on human CD3epsilon for bispecific 1 + 1 antibodies
according Example 2 with specificity for CD3 and HER2.

| | | Kinetics on human CD3e | | |
|---|---|---|---|---|
| Bispecific | CD3 specific | kₒₙ | k_{off} | K_{D} |
| antibody | antibody | [1/Ms] | [1/s] | [nM] |
| BissIg_18 opt_VH#2 | CD3-MABopt_VH#2 | 1.4E+05 | 3.4E−04 | 2.5 |
| BissIg_18 opt_VH#3 | CD3-MABopt_VH#3 | 9.5E+04 | 3.2E−04 | 3.4 |
| BissIg_18 opt_VH#4 | CD3-MABopt_VH#4 | 1.8E+05 | 3.3E−04 | 1.9 |
| BissIg_18 opt_VH#5 | CD3-MABopt_VH#5 | 1.6E+05 | 2.9E−04 | 1.9 |
| BissIg_18 opt_VH#1 | CD3-MABopt_VH#1 | 2.5E+05 | 4.4E−03 | 17* |
| BissIg_18 opt_VL | CD3-MABopt_VL | 1.5E+05 | 4.1E−03 | 28* |
| BissIg_18_opt_cc#2 | CD3-MABopt_cc#2 | 3.9E+05 | 1.6E−03 | 4.2 |
| BissIg_18_opt_cc#3 | CD3-MABopt_cc#3 | 2.4E+05 | 1.1E−03 | 4.4 |
| BissIg_18_opt_cc#4 | CD3-MABopt_cc#4 | 5.3E+05 | 1.4E−03 | 2.6 |
| BissIg_18_opt_cc#5 | CD3-MABopt_cc#5 | 3.2E+05 | 6.2E−04 | 1.9 |
| BissIg_18_opt_cc# | CD3-MABₒₚₜ_{_{—}}_{cc} | 1.2E+06 | 1.1E−03 | 0.9* |

*heterogenous binding

### Example 2.2: Cell Binding

Bispecific antibodies of Example 2 comprising affinity improved or cross-cloned CD3 specific antibodies according to the present disclosure were tested for their ability to bind to Jurkat (CD3+) and J.RT3-T3.5 (CD3−) cells. Target cells were mixed with the bispecific antibodies serially diluted (final concentration: 0.1 nM-200 nM) in D-PBS (Gibco) containing 3% fetal bovine serum (Sigma, #F7524) and incubated for 1 h on ice. Cells were washed 2 times in D-PBS (Gibco) containing 3% fetal bovine serum (Sigma, #F7524) and 0.02% Sodium acid. Bispecific antibodies were detected using AlexaFluor 647 labeled goat anti-Human IgG, F(ab′)2 Fragment specific (Jackson Immuno Research Cat #109-606-097). Antibody staining was measured using FACS Array (Beckton Dickinson) or IntelliCyt iQue flow cytometer and analyzed in FlowJo or ForeCyt (IntelliCyt) softwares, respectively. EC₅₀ values were calculated using 4-parameter non-linear regression analysis in Prism software (GraphPad Software Inc.).

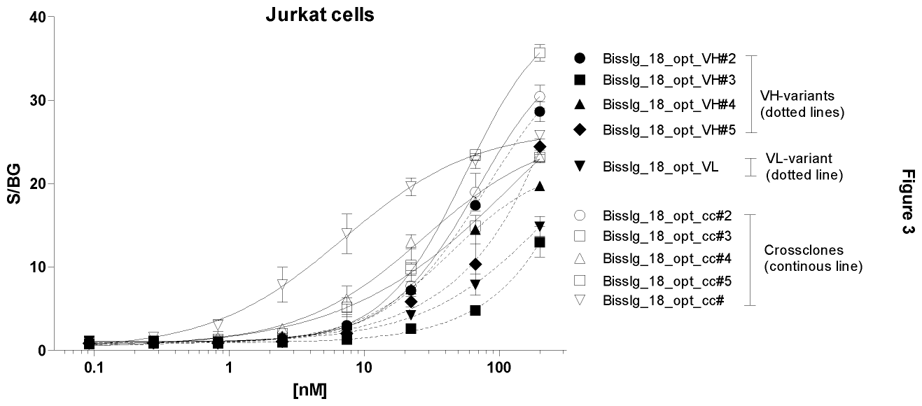

Table 20 and FIG. 3 summarizes cell binding of tested bispecific antibody preparations comprising affinity matured or cross-cloned CD3 specific antibody variable domains according to the present disclosure. Overall, CD3 specific cross-clones revealed stronger binding to Jurkat cells when compared to the solely affinity matured counterparts. In particular, cross-clone CD3-MABopt_cc (SEQ ID NO: 14 and SEQ ID NO: 20) exhibited strongest binding to Jurkat cells. Binding to the CD3 negative cell line J.RT3-T3.5. was observable for two affinity improved CD3 specific antibodies (CD3-MABopt_VH #2 and CD3-MABopt_VH #5) and their two cross-cloned counterparts (CD3-MABopt_cc #2 and CD3-CD3-MABopt_cc #5).

TABLE 20
Table 20: Cell binding (EC₅₀ values) of bispecific antibodies according Example
2 with specificity for HER2 and CD3 comprising affinity matured or cross-
cloned CD3 specific binding antibodies of the present disclosure.

| | | Cell Binding | |
|---|---|---|---|
| Bispecific | CD3 specific | Jurkat | J.RT3-T3.5 |
| antibody | antibody | EC₅₀ [nM] | binding @200 nM |
| BissIg_18_opt_VH#2 | CD3-MABopt_VH#2 | >50 | yes |
| BissIg_18_opt_VH#3 | CD3-MABopt_VH#3 | >100 | no |
| BissIg_18_opt_VH#4 | CD3-MABopt_VH#4 | 44 | no |
| BissIg_18_opt_VH#5 | CD3-MABopt_VH#5 | >50 | yes |
| BissIg_18_opt_VH#1 | CD3-MABopt_VH#1 | 9 | no |
| BissIg_18_opt_VL | CD3-MABopt_VL | 18/>100 | no |
| BissIg_18_opt_cc#2 | CD3-MABopt_cc#2 | >50 | yes |
| BissIg_18_opt_cc#3 | CD3-MABopt_cc#3 | >50 | no |
| BissIg_18_opt_cc#4 | CD3-MABopt_cc#4 | >20 | no |
| BissIg_18_opt_cc#5 | CD3-MABopt_cc#5 | >50 | yes |
| BissIg_18_opt_cc# | CD3-MABₒₚₜ_{_{—}}_{cc} | 7 | no |

### Example 2.3 Jurkat NFAT Reporter Gene Cell Assay

For the evaluation of the functional activity of the bispecific antibodies of Example 2, Jurkat cells (ATCC #TIB-152) transiently transfected with an NFAT reporter gene construct were used as surrogate effector cells. As HER2 positive target tumor cell lines, SKOV-3 (ATCC® HTB-77™) and MCF-7 (ATCC® HTB-22™) were used. The assay was conducted as essentially described in Example 1.5. The following growth media were used for maintenance of the cell lines: Jurkat: RPMI-1640+L-Glutamine (Thermo Fisher, #21875-034) supplemented with 10% FCS (Sigma, #F7524); SKOV-3: McCoys 5a (Gibco, #26600), supplemented with 10% FCS (Sigma #F7524) MCF-7: DMEM (Gibco #10938), supplemented with +10% FCS (Sigma #F7524)+1× Glutamax (Gibco #35050-061)+1× Sodium Pyruvat (Gibco #11360-039). SKOV-3 and MCF-7 cells were diluted in growth medium to a density of 4E+05 cells/ml. 100 μl cell suspension corresponding to 40,000 cells were seeded in each well of a tissue culture treated 96 well plate (Corning, #3917) and incubated overnight in a humidified incubator at 37° C. and 5% CO₂. Jurkat cells were resuspended in growth medium to a concentration of 2.5E+05 cells/ml. Transfection components pGL4.30[luc2P/NFAT-RE/Hygro] reporter gene vector (Promega #9PIE848), OptiMEM-I medium (Life Technologies, #31985-047) and TransIT-LT1 transfection reagent (Mirus, #MIR2304) were incubated for 15 min at RT, then added to the Jurkat cell suspension and incubated for 17 h in a humidified incubator at 37° C. and 5% CO₂. Jurkat cells were harvested and resuspended in growth medium at a concentration of 1.2E+06/ml. Medium was removed from coated target cells and replaced by 50 μl Jurkat cell suspension corresponding to 60,000 cells per well. Bispecific antibodies were serially diluted in Jurkat growth medium. 50 μl antibody dilution was added to each well resulting in a final concentration range of 50 nM to 0.01 nM (4 step dilution). Assay plates were incubated for 5 h in a humidified incubator at 37° C. and 5% CO₂. Bright-Glo™ Reagent (Promega, #E2620) was reconstituted according to manufacturer's instructions. Assay plates and reagent were equilibrated at room temperature. 100 μl of the Bright-Glo™ reagent was added to each well of the assay plate and mixed. Luminescence was measured using an InfiniteM1000 Pro plate reader (Tecan).

Table 21 summarizes activation of the Jurkat cells/NFAT reporter system mediated by the bispecific antibodies of the present disclosure. Again, bispecific antibody Bissig_18_opt_cc #comprising cross-clone antibody CD3-MABopt_cc (SEQ ID NO: 14 and SEQ ID NO: 20) exhibited strongest activation of the Jurkat cells/NFAT reporter system in the presence of both, SKOV-3 and MCF-7 cells with EC₅₀ values less than 0.2 nM.

TABLE 21
Reporter Gene Assay with bispecific antibodies according
to Example 2 with specificity for HER2 and CD3 comprising
affinity matured or cross-cloned CD3 specific binding
domains of the present disclosure.

| | | Reporter gene assay | |
|---|---|---|---|
| Bispecific | CD3 specific | SKOV3 | MCF-7 |
| antibody | antibody | EC₅₀ [nM] | EC₅₀ [nM] |
| BissIg_18 opt_VH#2 | CD3-MABopt_VH#2 | 1.18 | >5 |
| BissIg_18 opt_VH#3 | CD3-MABopt_VH#3 | 1.62 | 0.85 |
| BissIg_18 opt_VH#4 | CD3-MABopt_VH#4 | 0.89 | 2.04 |
| BissIg_18 opt_VH#5 | CD3-MABopt_VH#5 | 1.73 | >5 |
| BissIg_18 opt_VH#1 | CD3-MABopt_VH#1 | 0.18 | 0.33 |
| BissIg_18 opt_VL | CD3-MABopt_VL | 0.81/2.18 | 0.44/0.48 |
| BissIg_18_opt_cc#2 | CD3-MABopt_cc#2 | 0.61 | 0.39 |
| BissIg_18_opt_cc#3 | CD3-MABopt_cc#3 | 0.04 | 0.43 |
| BissIg_18_opt_cc#4 | CD3-MABopt_cc#4 | 0.33 | <0.5 |
| BissIg_18_opt_cc#5 | CD3-MABopt_cc#5 | 1.15 | 0.39 |
| BissIg_18_opt_cc# | CD3-MABₒₚₜ_{_{—}}_{cc} | 0.07 | <0.2 |

“>” = incomplete titration/bad fit

### Example 2.4: Cytotoxicity Assays with Bispecific Antibodies

Bispecific antibodies according Example 2 were tested for their ability to mediate T cell dependent killing of the HER2 expressing tumor cell lines SKBR3, MCF-7 and the HER2 negative cell line MDA-MB-468. The assay was conducted as described in Example 1.6. As effector cells, either human PBMCs (for SKBR3) cells or purified human T cells (for MCF-7 cells) were used. Preparation and purification of human PBMCs and human T cells was done as described in Example 1.4. 5000 SKBR-3, MDA-MB-468 or MCF-7 cells were seeded in black 96 well assay plates (Corning, #3340) and incubated over night at 37° C. and 5% CO₂. CellToxGreen dye (Promega, #G8731), serially diluted bispecific antibody constructs and 100.000 purified PBMCs for SKBR-3 cells (target/effector ratio of 1:20) or 50.000 purified T cells for MCF-7 cells (target/effector ratio of 1:10), all diluted in assay medium were added to the cells and incubated for 72 h at 37° C. and 5% CO₂. Cytotoxic activity was assessed after 72h by measuring incorporated CellToxGreen fluorescence at 485 nm excitation and 535 nm emission using a Tecan Infinite F500 device. EC₅₀ values were calculated using 4-parameter non-linear regression analysis in Prism software (GraphPad Software Inc.).

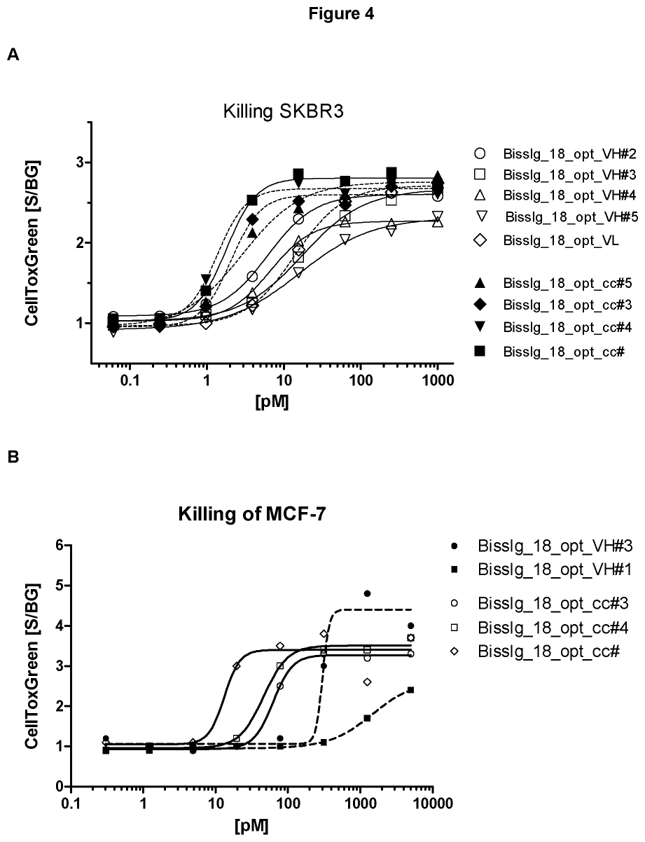

Table 22 and FIG. 4 (one doner) summarizes T cell redirected killing of the cancer cell lines mediated by the bispecific antibodies of the present disclosure. Bispecific antibodies comprising cross-cloned CD3 specific antibodies clearly showed superior killing of both, SKBR3 cells and MCF-7 cells. Of these, bispecific antibody BissIg_18_opt_cc #comprising cross-clone antibody CD3-MABopt_cc (SEQ ID NO: 14 and SEQ ID NO: 20) exhibited best killing activity of both cancer cell lines. Weak cell killing of the HER2 negative cell line MDA-MB-468 at higher tested antibody concentration was observable for cross-clone CD3-MABopt_cc #4 (data not shown).

TABLE 22
T cell mediated killing assay with bispecific antibodies according Example
2 with specificity for HER2 and CD3 comprising affinity matured or
cross-cloned CD3 specific antibodies of the present disclosure.

| Bispecific | CD3 specific | SKBR3 [PBMCs] | MCF-7 [T cells] |
|---|---|---|---|
| antibody | antibody | IC₅₀ [pM] | IC₅₀ [pM] |
| BissIg_18_opt_VH#2 | CD3-MABopt_VH#2 | n.t. | n.t. |
| BissIg_18_opt_VH#3 | CD3-MABopt_VH#3 | n.t. | 146.9/>300* |
| BissIg_18_opt_VH#4 | CD3-MABopt_VH#4 | n.t. | n.t. |
| BissIg_18_opt_VH#5 | CD3-MABopt_VH#5 | n.t. | n.t. |
| BissIg_18_opt_VH#1 | CD3-MABopt_VH#1 | 15.0 | 209.7/inc. Tit. |
| BissIg_18_opt_VL | CD3-MABopt_VL | 12.6/13.9 | n.t. |
| BissIg_18_opt_cc#2 | CD3-MABopt_cc#2 | 1.9 | n.t. |
| BissIg_18_opt_cc#3 | CD3-MABopt_cc#3 | 2.1 | 28.9/62.7 |
| BissIg_18_opt_cc#4 | CD3-MABopt_cc#4 | 1.4 | 28.9/46.1 |
| BissIg_18_opt_cc#5 | CD3-MABopt_cc#5 | 2.9 | n.t. |
| BissIg_18_opt_cc# | CD3-MABₒₚₜ_{_{—}}_{cc} | 1.8 | <10*/13.2 |

*bad fit;
n.t. = not tested

### Example 2.5: T Cell Activation in the Absence of Target Cancer Cells (High-Density PBMC Assay)

Bispecific antibodies of Example 2 were tested for their ability to activate human T cells derived from human blood samples of three different donors in the absence of target cancer cells. Assays were carried out under high PBMC density pre-culture conditions as suggested by Romer and colleagues (R6mer et al., BLOOD, 22 Dec. 2011, VOLUME 118, NUMBER 26, PAGE 6772-6781). Human PBMCs were prepared and purified as described before (see Example 1.4). T cells were resuspended to a density of 1E+07 cells/mL in RPMI 1640 medium (Gibco, #31870-025) supplemented with GlutaMax (Gibco, #35050-038), non-essential amino acids (Gibco, #11140-035), HEPES buffer solution (Gibco #15630-056), sodium pyruvate (Gibco, #11360-039), ß-mercaptoethanol, Penicillin/Streptomycin (Gibco #15140-122) and human serum (Sigma, #H4522) and incubated for 48 h at 37° C. and 5% CO₂. Following high-density preincubation, 200,000 PBMCs in medium were mixed with an equal volume of serially diluted antibodies (final concentration: 1000 nM, 200 nM, and 40 nM) in medium and incubated for 24 h at 37° C. and 5% CO₂. As positive control, the commercial available murine IgG antibody OKT3 was used. Activation of T cells was assessed by evaluation of upregulation of CD69 expression on CD3 positive lymphocytes. For this, PBMCs were stained with antibodies to CD3 and CD69 conjugated to BV/PE and APC respectively (Biolegend, #300434, #12003742, #310910). Antibody staining was measured using NovoCyte 3000 flow cytometer (Acea Biosciences, Inc.) and analyzed using FlowJo software.

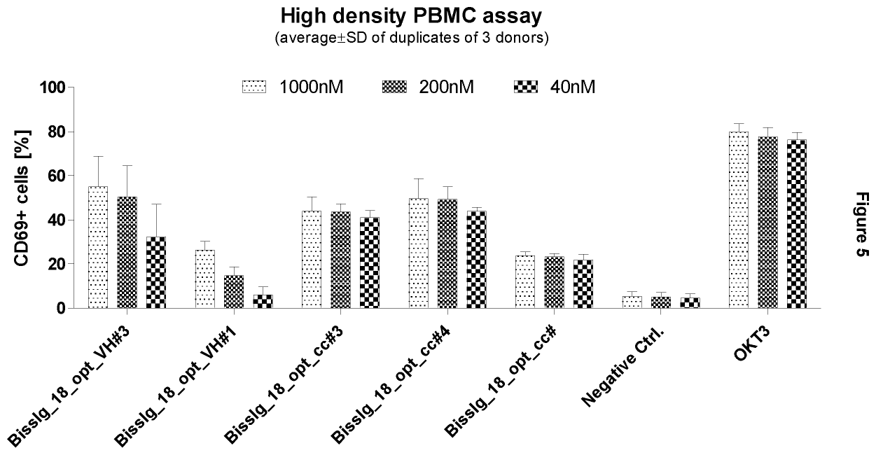

FIG. 5 shows average exemplary results of the T cell activation experiments for human CD8⁺ T cells obtained from 3 donors. As expected, strong upregulation of CD69 expression was observable for the positive murine control IgG OKT-3. A donor dependent activation of CD8⁺ T cells could be observed for all tested bispecific antibodies comprising affinity optimized or cross-cloned CD3 specific antibodies. Of these, bispecific antibodies comprising either CD3-MABopt_VH #1 (SEQ ID NO: 14 and SEQ ID NO: 26) or its cross-cloned counterpart CD3-MABopt_cc (SEQ ID NO: 14 and SEQ ID NO: 20) exhibited lowest levels of human T cell activation in the absence of target cancer cell lines.

### Example 2.6: Summary of Affinity Improved and Cross-Cloned CD3 Specific Antibodies

The strongly affinity improved cross-clone CD3-MAB_{opt_cc} (SEQ ID NO: 14 and SEQ ID NO: 20) was identified as most potent and most safe CD3 specific antibody derived from the affinity maturation campaign of the grandparent antibody CD3-MAB_{GP}.

CD3-MABopt_cc shows favorable binding to human and cynomolgus CD3 and mediates excellent cytotoxic activity on HER2 low, mid and high expressing cancer cell lines, no killing of HER2 negative cancer cell lines and only low levels of T cell activation in the absence of target cells when tested in a bispecific antibody format of Example 2.

### Example 3: Conversion of Optimized CD3 Specific Antibody CD3-MABopt-cc into an Improved 2+1 Fab₂-Fv-Fc Antibody Format

### Example 3.1: Preparation, Production and Characterization of Linker Optimized Trivalent Bispecific Antibodies

Bispecific Fab₂-Fv-Fc antibodies were generated in vitro using the bispecific antibody platform technology as described in WO 2020/115115 which is incorporated herein in its entirety. This bispecific antibody format is built from an aglycosylated monoclonal human IgG1 antibody backbone incorporating one extra antibody Fv fragment inserted between the Fc region and the two Fab arms of a regular human IgG1 molecule. A basic structure of such a bispecific antibody is provided in FIG. 2. Such format provides the advantage of bivalent binding to a target cell surface antigen, such as a tumor associated antigen but monovalent binding to CD3, expressed on T cells. This format also allows for a short distance between a target cell and a cytotoxic T cell once bridged via the bispecific antibody. This narrow immunological synapse leads to an efficient killing of the target cell by the recruited cytotoxic T cell.

### Example 3.2: Linker Optimization

The choice of the right peptide linkers (in terms of amino acid sequence and length) connecting the individual components of the bispecific antibody format is of particular importance as these linkers determine the flexibility of the extra Fv fragment and its distance to the binding regions of each of the two Fab arms of the bispecific antibody. To further optimize the previously disclosed peptide linkers in WO 2020/115115, respective linkers were now elongated and sequence optimized.

### N-Terminal Linkers to the CD3 Specific Fv Fragment

The fusion of the C-terminus of each Fab heavy chain to either the N-terminus of the VH or VL of the incorporated CD3 specific Fv fragment was achieved by using one of the following peptide linkers:

(SEQ ID NO: 30)
a) 9 mer glycine-serine linker (GGS)₃: GGSGGSGGS
(SEQ ID NO: 31)
b) 20 mer (G₄S)₄ linker: GGGGSGGGGSGGGGSGGGGS
(SEQ ID NO: 33)
c) 20 mer PAPDA linker: AQPAAPAPDAHEAPAPAQGS,
or
(SEQ ID NO: 32)
d) 20 mer PAH linker: AHPAAPAPAHPAAPAPAHGH

### C-Terminal Linkers to the CD3 Specific Fv Fragment

The fusion between the C-terminus of each of the VH and VL domain of the CD3 specific Fv fragment to the N-terminus of either one of the two Fc region subunits was achieved by using one of the following peptide linkers:
i) in case of fusing the VL domain of the CD3 specific Fv fragment to one of the two Fc region subunits:
   a) the first 5 amino acid residues of a CL, constant domain: PKAAP (SEQ ID NO: 36)
   b) 20mer PAPDA linker: AQPAAPAPDAHEAPAPAQGS (SEQ ID NO: 33), or
   c) the first 20 amino acid residues of a CL, constant domain: PKAAPSVTLFPPSSEELQAN (SEQ ID NO: 34) or
ii) in case of fusing the VH domain of the CD3 specific Fv fragment to one of the two Fc region subunits:
   a) first 6 amino acid residues of a CH1 constant domain: ASTKGP (SEQ ID NO: 37),
   b) 20mers PAPDA linkers: AQPAAPAPDAHEAPAPAQGS (SEQ ID NO: 33), or
   c) first 20 amino acid residues of a CH1 constant domain: ASTKGPSVFPLAPSSKSTSG (SEQ ID NO: 35)

A summary of the tested Linker Combinations are listed in Table 23. FIG. 2 depicts the different peptide linkers used to connect the different components of the bispecific antibody format of Example 3.

TABLE 23
Linker Combinations used to connect the VH and VL domain of the incorporated extra CD3 specific
Fv fragment to the Fab arms and Fc region subunits in the bispecific Fab₂-Fv-Fc antibody format.

| | VL domain of CD3 specific | | | | VH domain of CD3 specific | | | |
|---|---|---|---|---|---|---|---|---|
| | Fv fragment (knob-chain) | | | | Fv fragment (hole chain) | | | |
| Linker | N-terminal | SEQ | C-terminal | SEQ | N-terminal | SEQ | C-terminal | SEQ |
| Combination | linker | ID NO: | linker | ID NO: | linker | ID NO: | linker | ID NO: |
| P (Parental)* | (GGS)₃ | 30 | 5aa-Cλ | 36 | (GGS)₃ | 30 | 6aa-CH1 | 37 |
| 1 | (G₄S)₄ | 31 | 5aa-Cλ | 36 | (G₄S)₄ | 31 | 6aa-CH1 | 37 |
| 2 | (G₄S)₄ | 31 | 20aa-PAPDA | 33 | (G₄S)₄ | 31 | 20aa-PAPDA | 33 |
| 3 | (G₄S)₄ | 31 | 20aa-Cλ | 34 | (G₄S)₄ | 31 | 20aa-CH1 | 35 |
| 4 | 20aa-PAH | 32 | 20aa-Cλ | 34 | 20aa-PAH | 32 | 20aa-CH1 | 35 |
| 5 | 20aa-PAPDA | 33 | 20aa-Cλ | 34 | 20aa-PAPDA | 33 | 20aa-CH1 | 35 |
| 6 | (GGS)₃ | 30 | 20aa-Cλ | 34 | (GGS)₃ | 30 | 20aa-CH1 | 35 |

*as originally disclosed in WO 2020/115115

All C-terminal linkers were further C-terminally elongated by a portion of an human IgG1 hinge sequence DKTHTCPPCP (SEQ ID NO: 38). The use of the truncated human IgG1 hinge sequence allowed for a further stabilization of the heterodimeric molecule via the formation of interchain-disulfide bridges between the two polypeptide chains comprising the Fab heavy chains. The Fc region was modified by introducing mutations into the CH3 domain of each Fc region subunit according to the “knob-into-holes” technology. Thereby, the polypeptide comprising one mutated CH3 domain is forced to heterodimerize with the other polypeptide comprising the other CH3 domain, which is engineered in a complementary manner. For HER2 binding, the VH and VL domain of “Trastuzumab” (HERCEPTIN®) (SEQ ID NO: 28 and SEQ ID NO: 29, respectively) as described by Baselga et al. 1998, Cancer Res 58(13): 2825-2831) were used. Trastuzumab and its method of preparation are described in U.S. Pat. No. 5,821,337. For CD3 binding, the VH and VL of the CD3 specific cross-clone CD3-MABopt-cc (SEQ ID NO: 14 and SEQ ID NO: 20, respectively) of Example 2 was used. A summary of the individual polypeptides forming the produced bispecific trivalent Fab₂-Fv-Fc antibodies employing the different Linker Combinations of Table 23 made in accordance with Example 3 is set forth in Table 13.

### Example 3.3: Gene Synthesis and Production

All nucleic acid sequences or desired gene segments were either generated by PCR using appropriate templates or were gene synthesized as linear DNA fragments with appropriate flanking regions (e.g. suitable restriction enzyme recognition sites, linker sequences) in-house or by an external provider. The nucleic acid sequences or gene segments flanked by singular restriction endonuclease cleavage sites were cloned into respective expression vectors (e.g. mammalian expression vectors) or sequencing vectors using standard molecular biology methods. When intended for use in mammalian expression vectors, all constructs were designed with a 5′-end DNA sequence coding for a leader peptide which targets proteins for secretion in eukaryotic cells. The DNA sequence of the subcloned gene fragments was confirmed by DNA by double strand sequencing. For expression of the bispecific antibodies, exponentially growing eukaryotic HEK293-6E cells were transfected with a mammalian vector expression system encoding all components of the bispecific antibodies, resulting in a 1:1:2 ratio of the two polypeptides comprising the two Fc region subunits and the 2 polypeptides comprising the Fab light chain of Trastuzumab, respectively. Cell culture supernatants were harvested on day 6 post transfection and subjected to standard Protein A affinity chromatography (MabSelect SURE|GE Healthcare). Buffer exchange was performed to 1× Dulbcecco's PBS (pH 7.2|Invitrogen) and samples were sterile filtered (0.2 μm pore size). Protein concentrations were determined by UV-spectrophotometry and purities of the constructs were analyzed under denaturing, reducing and non-reducing conditions using CE-SDS (LabChip GXII|Perkin Elmer|USA). HP-SEC was performed to analyze bispecific antibody preparations in native state. In addition, molecular mass and homogeneity of the bispecific antibodies was confirmed by mass spectrometry analysis.

Table 24 summarizes monomer content and volumetric yields of the different preparations obtained for the produced bispecific antibodies. All produced bispecific antibodies revealed comparable production characteristics.

However, all bispecific antibodies using CH1- or Cλ-derived peptide linkers revealed a very heterogenous mass pattern indicating potential O-linked glycosylation of these linkers. Only bispecific antibody BissIg_21 #2 using Linker Combination 2, i.e. a 20mer glycine-serine linker (SEQ ID NO: 31) in combination with the 20mer PAPDA linker (SEQ ID NO: 33) showed a homogenous mass pattern and no potential signs of 0-linked glycosylation.

TABLE 24
Quality control of mammalian produced bispecific Fab₂-Fv-Fc antibodies comprising
the same HER2 and CD3 binding domains but different Linker Combinations.

| Bispecific | Linker | SEC | Volumetric | Mass |
|---|---|---|---|---|
| antibody | Combination | Monomer | Yield | spectrometry |
| (Table 13) | (Table 23) | Content [%] | [mg/L] | analysis |
| BissIg_21#P | P | 92.3* | 61.7 | Heterogenous pattern - potential |
| | | | | O-linked glycosylation |
| BissIg_21#1 | 1 | 91.2* | 73.3 | Heterogenous pattern - potential |
| | | | | O-linked glycosylation |
| BissIg_21#2 | 2 | 84.0**/91.4*** | 81.3/58.3 | Homogenous pattern - no signs of |
| | | | | O- linked glycosylation |
| BissIg_21#3 | 3 | 91.3* | 70.0 | Heterogenous pattern - potential |
| | | | | O-linked glycosylation |
| BissIg_21#4 | 4 | 91.4* | 67.1 | Heterogenous pattern - potential |
| | | | | O-linked glycosylation |
| BissIg_21#5 | 5 | 91.9*/90.9*** | 61.3/48.2 | Heterogenous pattern - potential |
| | | | | O-linked glycosylation |
| BissIg_21#6 | 6 | 91.0* | 68.3 | Heterogenous pattern - potential |
| | | | | O-linked glycosylation |

### Example 3.4: K_{D} Determination Via Antibody Capture Setup

Affinity determination by determining kinetic rate constants was performed on an Octet HTX (FortéBIO, Sartorius AG) instrument. The different bispecific antibody preparations of Example 3 diluted in assay buffer (D-PBS, 0.05% (v/v) PS 20, 0.1% (w/v) BSA) were captured onto IgG-specific BLI sensors with a loading level of approx. 2 nm. For analysis, human CD3 epsilon antigen hCD3e(22-118)_F-chLys_avi (SEQ ID NO: 43) was diluted with assay buffer to concentrations ranging from 1.56 nM to 500 nM (serial 1:3 dilution). A blank sample with assay buffer was included for referencing, i.e. correcting for dissociation of captured antibody. The association phase was recorded for 180 s, followed by a dissociation phase of 300 s. The sensorgrams were fitted using Octet Data Analysis Software 10.0 (FortéBio, Sartorius AG) to determine kₒₙ and k_{off} rate constants (using a 1:1 binding model), which were used to calculate K₀.

Table 25 summarizes K_{D} values human CD3epsilon antigen for bispecific antibodies of the present Example using the different Linker Combinations of Table 23. A comparable CD3 binding was observed for all tested Linker Combinations.

TABLE 25
Affinities on human CD3epsilon antigen of bispecific
antibodies comprising the variable domains of CD3-
MABopt-cc but different Linker Combinations

| Bispecific | Linker | | | |
|---|---|---|---|---|
| antibody | Combination | kₒₙ | k_{off} | K_{D} |
| (Table 13) | (Table 23) | [1/Ms] | [1/s] | [nM] |
| BissIg_21#P | P | 3.04E+05 | 1.21E−03 | 4.0* |
| BissIg_21#1 | 1 | 2.67E+05 | 1.12E−03 | 4.2* |
| BissIg_21#2 | 2 | 2.66E+05 | 9.74E−04 | 3.7* |
| BissIg_21#3 | 3 | 2.46E+05 | 1.08E−03 | 4.4* |
| BissIg_21#4 | 4 | 2.14E+05 | 1.13E−03 | 5.3* |
| BissIg_21#5 | 5 | 1.92E+05 | 9.84E−04 | 5.1* |
| BissIg_21#6 | 6 | 2.87E+05 | 1.02E−03 | 3.6* |

*Slightly heterogenous binding; values represent best approximation to 1:1 binding

### Example 3.5: Cell Binding

Bispecific antibodies according Example 3 comprising the different Linker Combinations of Table 23 were tested for their ability to bind to CD3 positive Jurkat cells and to CD3 negative J.RT3-T3.5 cells. Jurkat and J.RT3-T3.5. cells were resuspended, counted and blocked and blocked in wash-buffer (DPBS+(Gibco)/3% FBS (Sigma)/0.02% Sodium acid) for 1 h on ice. Blocked cells were resuspended in bispecific antibodies serially diluted in wash buffer (final concentration: 500 nM-31 pM) and incubated for 1 h on ice. Cells were washed 2 times in wash-buffer. Bound antibodies were detected using AlexaFluor 647 labeled goat anti-Human IgG, F(ab′)₂ Fragment specific (Jackson Immuno Research Cat #109-606-097). EC₅₀ values were calculated using 4-parameter non-linear regression analysis in Prism software (GraphPad Software Inc.)

Table 26 summarizes cell binding results (EC₅₀ values) of two independent experiments. Bispecific antibodies with specificity for HER and CD3 comprising the different Linker Combinations exhibited comparable binding to Jurkat cells when compared to the originally described Linker Combination P. No binding to the CD3 negative Jurkat cells (RT3-T3.5) could be observed (data not shown).

TABLE 26
Cell binding

| Bispecific | Linker | |
|---|---|---|
| antibody | Combination | Jurkat |
| (Table 13) | (Table 23) | EC₅₀ [nM] |
| BissIg_21#P | P | 61.5/83.7 |
| BissIg_21#1 | 1 | 54.8/53.9 |
| BissIg_21#2 | 2 | 71.7 |
| BissIg_21#3 | 3 | 89.2/58.2 |
| BissIg_21#4 | 4 | 43.2/64.1 |
| BissIg_21#5 | 5 | 98.3/132.7 |
| BissIg_21#6 | 6 | 60.9/64.1 |

### Example 3.6: Cytotoxicity Assays with Bispecific Antibodies Comprising Different Linker Combinations

Bispecific antibodies according Example 3 comprising different Linker Combinations were tested for their ability to induce T cell-mediated killing of HER2 expressing SKOV-3 and HER-2 negative MDA-MB468 cells. Human pan T cells from two donors were prepared and purified as described before (see Example 1.4). 5,000 HER2 expressing SKOV-3 cells were suspended in culture medium (SKOV-3: McCoy's 5A Medium (ThermoFisher, #26600), 10% FCS (Sigma, #F7524) seeded in black 96 well assay plates (Corning, #3340) and incubated over night at 37° C. and 5% CO₂. CellToxGreen dye (Promega, #G8731), serially diluted bispecific antibodies (0.3 nM-1.2 pM) and 50,000 purified T cells (target/effector ratio of 1:10), all diluted in assay medium comprising RPMI 1640 w/o Phenol red (Gibco, #32404-014), GlutaMAX and 10% fetal bovine serum, were added to the cells and incubated for 72 h at 37° C. and 5% CO₂. Cytotoxic activity was assessed by measuring incorporated CellToxGreen fluorescence at 485 nm excitation and 535 nm emission using a Tecan Infinite F500 device. EC₅₀ values were calculated using 4-parameter non-linear regression analysis in Prism software (GraphPad Software Inc.).

Table 27 summarizes average IC₅₀ values of T cell redirected killing of SKOV-3 cells mediated by the bispecific antibodies comprising different Linker Combinations. A dose dependent killing was confirmed for all bispecific antibodies. 5 Linker Combinations resulted in improved cell killing compared to the originally disclosed Linker Combination P (BissIg_21 #P). No HER2 independent killing of MDA-MB468 cells could be observed for any of the tested bispecific antibodies (data not shown).

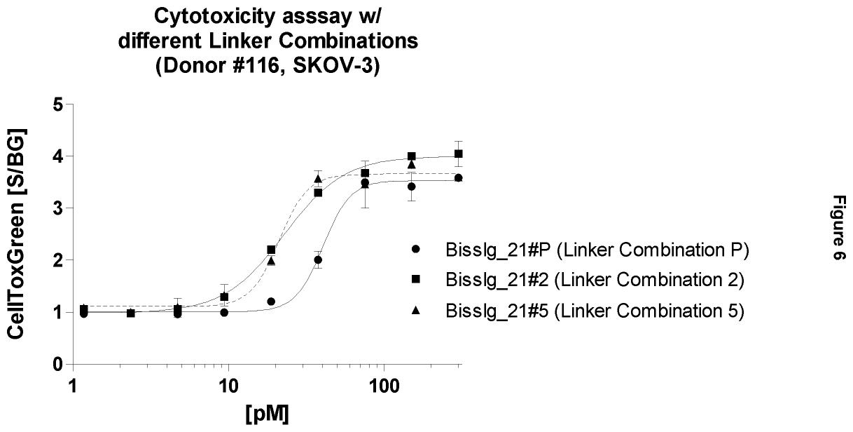

FIG. 6 shows exemplary SKOV-3 killing results mediated by the bispecific antibodies BissIg_21 #2 and BissIg_21 #5 comprising the most potent Linker Combination 2 and Linker Combination 5, in comparison to the originally disclosed Linker Combination P (BissIg_21 #P).

TABLE 27
T cell redirected killing of HER2 positive SKOV-3 cells

| Bispecific | Linker | Killing of |
|---|---|---|
| antibody | Combination | SKOV-3 cells |
| (Table 13) | Table 23 | IC₅₀ [nM]* |
| BissIg_21#P | P | 48** |
| BissIg_21#1 | 1 | 23 |
| BissIg_21#2 | 2 | 21 |
| BissIg_21#3 | 3 | 39 |
| BissIg_21#4 | 4 | 21 |
| BissIg_21#5 | 5 | 20** |
| BissIg_21#6 | 6 | 74 |

*average of 4 donors
**average of 6 donors

### Example 3.7: T Cell Activation in the Absence of Target Cancer Cells (High-Density PBMC Assay)

Bispecific antibodies of Example 3 comprising different Linker Combinations were tested for their ability to activate human T cells derived from human blood samples of three different donors in the absence of target cancer cells. The assay was essentially carried out as described in Example 2.5. Bispecific antibodies were tested at a final concentration of 1000 nM, 200 nM, 40 nM, 8 nM, 1.6 nM and 0.32 nM. As positive control, the commercial available murine IgG antibody OKT3 was used. Activation of T cells was assessed by evaluation of upregulation of CD69 expression on CD4+ and CD8+ T cells. For this, PBMCs were stained with antibodies to CD69, CD4 and CD8 conjugated with APC, PE, Pacific Blue, respectively (Biolegend, #310910/#300508/#300928). Antibody staining was measured using NovoCyte 3000 flow cytometer (Acea Biosciences, Inc.) and analyzed using FlowJo software.

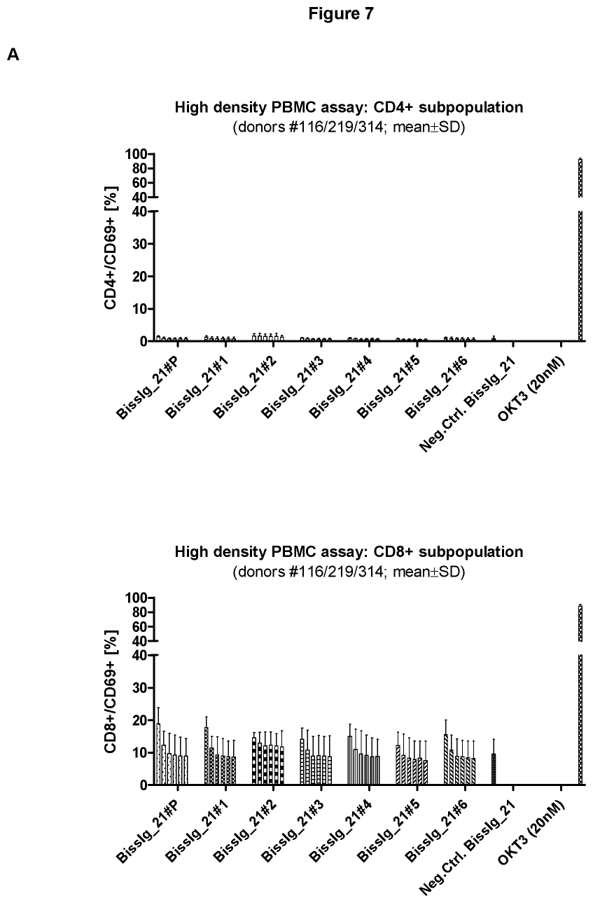

Average results of the T cell activation experiments obtained from 3 donors are shown in FIG. 7A for CD4+/CD8− T cells and FIG. 7B for CD4−/CD8+ T cells. As expected, strong upregulation of CD69 expression on CD4 positive and CD8 positive T cells was observable for the positive control IgG OKT-3. No activation of CD4+ T cells was observable for any of the tested bispecific antibodies in the absence of HER2 expressing target cells even at the highest tested concentration of 1 pM (decreasing antibody concentration from left to right). A slight donor dependent activation of CD8+ T cells could be observed for some of the tested Linker Combinations, but only at the highest tested antibody concentration of 1 pM. Most pronounced activation could be observed for the originally disclosed Linker Combination P (BissIg_21 #P), Linker Combination 1 (BissIg_21 #1) and Linker Combination 6 (BissIg_21 #6).

### Example 3.8: Summary Linker Optimization

All bispecific 2+1 Fab₂-Fv-Fc antibodies of Example 3 employing the new Linker Combinations revealed similar characteristics in production and binding to CD3 when compared to the bispecific antibody comprising the originally described Linker Combination P (BissIg_21 #P).

However, bispecific antibodies BissIg_21 #1, BissIg_21 #2, BissIg_21 #3, BissIg_21 #4 and BissIg_21 #5 comprising Linker Combination 1, 2, 3, 4 and 5, respectively, revealed improved efficacy in T cell mediated killing of SKOV-3 cells and less target-independent T cell activation when compared to BissIg_21 #P, comprising the originally described Linker Combination P. However, all bispecific antibodies comprising CH1 or Cλ-based peptide linkers revealed signs of O-glycosylation after mammalian production as determined by mass spectrometry analysis. Only the bispecific antibody BissIg_21 #2 comprising the Linker Combination 2 encompassing the elongated 20mer peptide linkers (G₄S)₄ (SEQ ID NO: 31) and 20aa-PAPDA (SEQ ID NO: 33) showed no signs of O-glycosylation. Accordingly, Linker Combination 2 was identified as the most preferred Linker Combination to be used in the improved bispecific 2+1 Fab₂-Fv-Fc antibody format of Example 3.

### Example 4: Generation of Deimmunized Variant Antibodies of Optimized CD3 Specific Antibody CD3-MAB_{opt-cc}

Deimmunized variants of the human CD3 specific antibodies of CD3-MAB_{opt-cc} of Example 2 having the VH of SEQ ID: 14 and the VL of SEQ ID NO: 20 were prepared.

This antibody is characterized by germline encoded human framework regions in its VH and VL as well as germline encoded LCDR1 and LCDR2 regions. The following examples describe the deimmunization of the non-human germline encoded HCDR1, HCDR2, HCDR3 and LCDR3 regions of CD3-MAB_{opt-cc}.

### Example 4.1: Identification of (Potential) T Cell Epitopes, H-Lines and Hotspots in the CDR Regions of CD3-MAB_{opt-cc}

The amino acid sequences of the VH (SEQ ID: 14) and VL (SEQ ID NO: 20) of CD3-MAB_{opt-cc} were analyzed for potential T cell epitopes, H-lines and Hotspots by using the in silico T cell epitope screening tool (Lonza, The Epibase™, Epibase Version: v3.0). Its basic computing routine is described in WO 2003/105058 (which is incorporated herein in its entirety). This screening tool allows for the identification of potential T cell epitopes in biotherapeutic proteins, such as antibodies. The tool uses structural characteristics of the HLA receptor along with experimentally determined binding affinities to predict potential peptide/HLA binding, a condition necessary for T cell activation.

For analysis, the whole VH or VL sequence is splitted into overlapping 10mer peptides (referred herein as “analyzed 10mer peptides), each of which is shifted by one amino acid.

Potential peptide/HLA binding for each 10mer peptide was determined for the HLA class II allotypes of the major Caucasian DRB1 alleles. Human antibody germline encoded sequence regions were excluded from analysis.

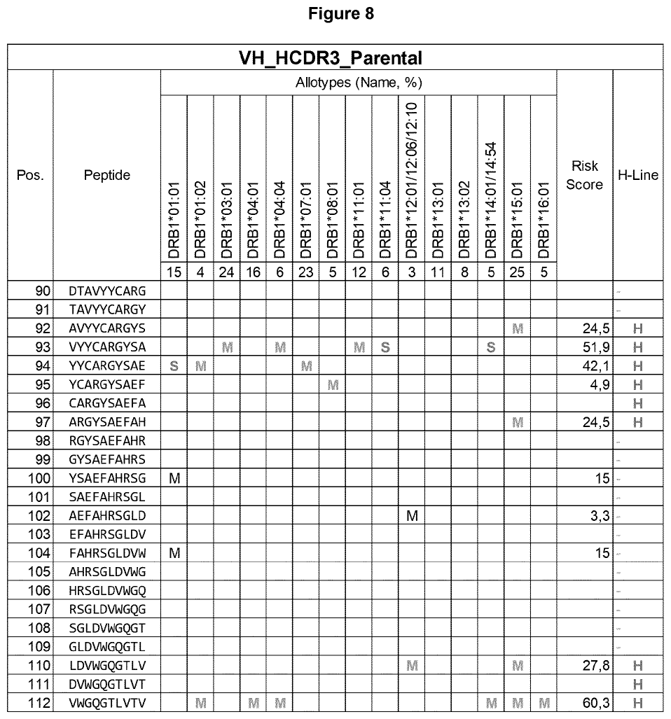

Result of Epibase™ Analysis Results of the in silico “searching” process revealed the presence of 6 Hotspots within the HCDR1, HCDR2, HCDR3 region of CD3-MAB_{opt-cc} and one Hotspot in the LCDR3 region of CD3-MAB_{opt-cc}. Hotspots reflects an accumulation of neighboring/adjacent T cells epitopes. Such Hotspots were identified based on an “4 over 3” algorithm as detailed in figure legend 8 and FIG. 8 and requires that at least 4 allotypes of the DRB1 allele bind to at least two of three consecutive analyzed 10mer peptides with moderate (M) or strong (S) affinity. In addition, at most one 10mer peptide, that is not identified as a T cell epitope, can be part of a Hotspot. Consequently, not every identified T cell epitope must be part of a Hotspot (see for instance peptides 100, 102 or 104 of FIG. 4.) On the other hand, a peptide not identified as a T cell epitope, may be still part of a Hotspot (see for instance peptide 96 or peptide 111 of FIG. 4). Each 10mer peptide being part of a Hotspot is defined as a H-line. An Absolut Risk Score for an Hotspot can be calculated as the sum of Risk Scores determined for each T cell epitope within a Hotspot and is provided as a “H-Score”.

Table 28 and Table 29 provide a summary of the overall determined immunogenicity risk parameters for the VH and VL of CD3-MAB_{opt-cc}. As the LCDR1+2 regions are germline encoded sequences, no Hotspots were allocated to these regions.

TABLE 28
Epibase screening results for the VH of antibody CD3-MAB_{opt-cc}

| | |
|---|---|
| Absolute Score | 652.5 |
| Absolute Hotspots | 6 |
| Absolute H-Score | 580 |
| Absolute H-Lines | 25 |
| Absolute T cell epitopes | 23 |

TABLE 29
Epibase screening results for the VL of CD3-MABopt-cc

| | |
|---|---|
| Absolute Score | 188.8 |
| Absolute Hotspots | 1 |
| Absolute H-Score | 188.8 |
| Absolute H-Lines | 7 |
| Absolute T-cell epitopes | 6 |

An exemplary detailed screening analysis for the HCDR3 region of CD3-MAB_{opt-cc} is shown in FIG. 8 (for detailed explanation refer to figure legend of FIG. 8). In sum, for the HCDR3 region, 2 Hotspots encompassing 7 T cell epitopes and 9 H-Lines with an H-Score of 236 were identified. For the HCDR2 region, 3 Hotspots encompassing 10 T cell epitopes and 12 H-Lines with an H-Score of 254.1 For the HCDR1 region, 1 Hotspot encompassing 3 T cell epitopes and 4 H-Lines with an H-Score of 89.9 were identified. For the LCDR3 region, 1 Hotspot encompassing 6 T cell epitopes and 7 H-Lines with an H-Score of 188.8 were identified.

### Example 4.2: Generation of Deimmunized Variant VH and VL Sequences of Antibody CD3-MAB_{opt-cc} by Single Amino Acid Substitutions in its CDR Regions

In order to remove or reduce the previously identified Hotspots, T cell epitopes and/or H-lines from the CDR regions of CD3-MAB_{opt-cc}, an in silicic Epibase™ mutation analysis was performed, wherein each amino acid position in the VH and VL of CD3-MAB_{opt-cc} was virtually substituted/randomized with each of the natural occurring amino acid residue except for cysteine, proline and histidine. Amino acid substitutions which preferably resulted in a decrease of T cell epitopes of >=2 within an identified Hotspot and preferably allowed of making conservative amino acid substitutions, such as to select for amino acids with similar charge or polarity, were selected for gene synthesis of deimmunized variant VH and VL antibody sequences. In addition, care was taken not to introduce potential posttranslational modification sites (“PTM motifs”) into the CDR regions.

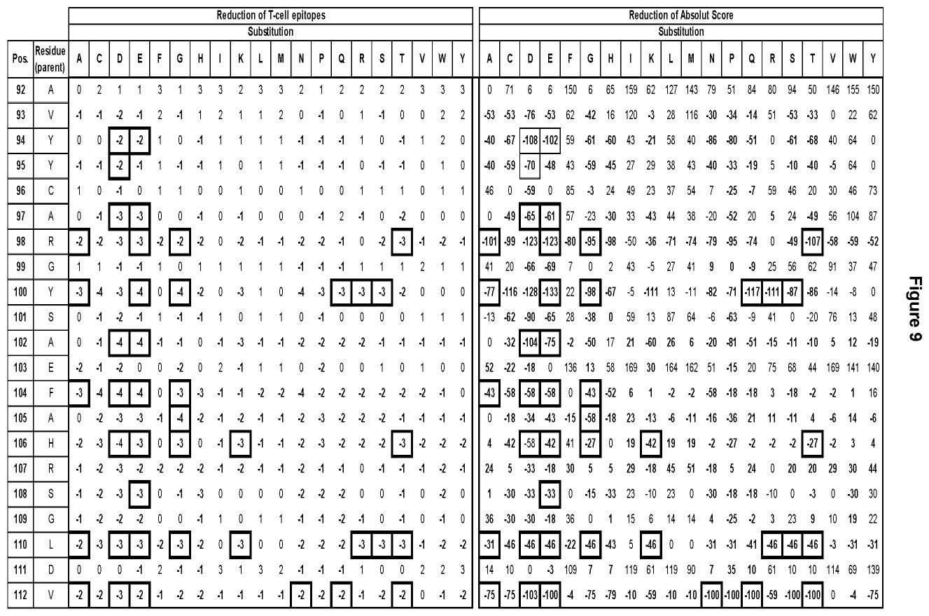

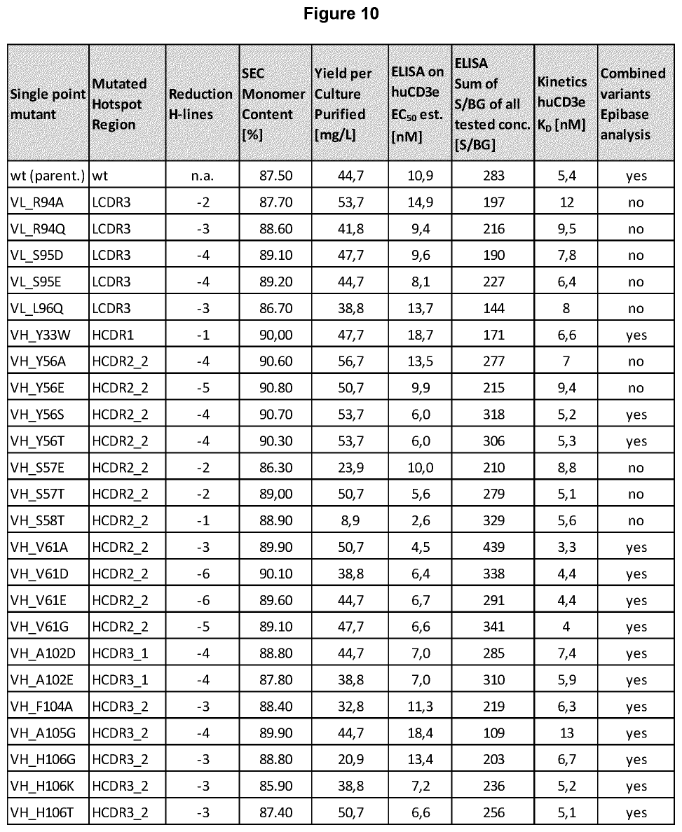

FIG. 9 depicts the impact of single amino acid substitutions at each HCDR3 position of CD3-MAB_{opt-cc} on the number of T cell epitopes (left panel of FIG. 4) and corresponding Absolut Risk Score (right panel of FIG. 4) compared to the unmodified parental sequence. FIG. 10 depicts the impact of preferred single amino acid substitutions on the number of T cell epitopes for 24 actually produced and characterized bispecific antibodies (as described below) comprising the VH or VL single point mutant variants.

In total, 92 physical VH or VL single point variants of CD3-MAB_{opt-cc} were generated by a PCR based mutagenesis strategy. Briefly, linear DNA fragments were produced by PCR with suitable oligonucleotides harboring the favored mutations and homologous overlapping sequences and subsequently cloned into the corresponding mammalian bispecific antibody expression vectors encoding the bispecific 2+1 Fab₂-Fv-Fc antibody format as described in Example 3 with specificity for HER2 and CD3. VH variants were combined with the parental unmodified VL of CD3-MAB_{opt-cc} (SEQ ID NO: 20), whereas VL variants were combined with the parental unmodified VH of CD3-MAB_{opt-cc} (SEQ ID NO: 14). Bispecific antibody were produced as previously described in Example 3.

Overall, approx. 50% of the bispecific antibodies could be produced with an acceptable monomer content of >85% (see Table 30). FIG. 10 summarizes yields and final monomer content of bispecific antibody preparation comprising the above referenced 24 preferred single point variants

TABLE 30
Summary of produced bispecific antibodies comprising CDR single
point variants of CD3-MABopt-cc with an monomer content >85%
as determined by analytical size exclusion chromatography.

| | Number of bispecific antibody preparations with a |
|---|---|
| Single point variants | monomer content >85% in reference to the total |
| in CDR region | number of produced bispecific antibodies |
| HCDR1 | 5/6 |
| HCDR2 | 13/22 |
| HCDR3 | 18/42 |
| LCDR3 | 10/23 |

Surprisingly, it was found that amino acid exchanges (even when being conservative) at particular CDR positions had a strong negative influence on the producibility of the bispecific antibodies, i.e. resulted in an increased aggregation propensity. Table 31 depicts substituted amino acid positions within the CDRs of CD3-MAB_{opt-cc} which resulted in a prominent decrease in monomer content of the corresponding produced bispecific antibodies.

TABLE 31
Substituted amino acid positions within the CDRs of CD3-
MAB_{opt-cc} which resulted in remarkable increase in aggregate
formation of bispecific antibody preparations.

| | | # variants | SEC monomer |
|---|---|---|---|
| Mutated aa- | Mutated | produced | content range |
| position within | CDR | per | [%] for |
| CDR region | region | position | all <77% |
| W47 | HCDR2 | 1 | 64 |
| V48 | | 2(+1) | 7-8 |
| Y94 | HCDR3 | 2 | 8-10 |
| Y95 | | 1 | n.a. |
| R98 | | 3(+1) | 61-76 |
| L110 | | 6(+2) | 12-35 |
| V112 | | 5(+1) | 17-70 |
| Y87 | LCDR3 | 3 | 20-24 |
| Y88 | | 1 | 25 |
| G91 | | 1 | 64 |
| G98 | | 1 | 65 |

### Example 4.3: ELISA Binding

46 produced bispecific single point variant antibodies according Example 4.2 with a monomer content of >85% were characterized for ELISA binding to human CD3epsilon antigen (hCD3e(22-118)_F-chLys_avi (SEQ ID NO: 43) coated on Maxisorp plates (Nunc, #460518). Final antibody concentrations were set to 50, 10, 2, 0.4 nM, respectively. Bound antibodies were detected using an alkaline phosphatase-conjugated detection antibody directed against human F(ab′)2 fragment (Jackson Immuno Research, #109-055-097). EC₅₀ values were calculated using 4-parameter non-linear regression analysis in Prism software (GrapPad Software Inc.).

FIG. 10 summarizes ELISA EC₅₀ estimates on human CD3epsilon antigen for 24 bispecific single point variant antibodies revealing similar or even better binding to CD3 when compared to the parental antibody CD3-MAB_{opt-cc} (provided are EC₅₀ estimates+ sum of signal to background ratios over all tested antibody concentration).

### Example 4.4: K_{D} Determination Via Antibody Capture Setup

Affinity determination by determining kinetic rate constants was performed for the bispecific single point variant antibodies as previously described in Example 3.4. The different bispecific antibody samples diluted in assay buffer (1% BPBS+0.05% Tween 20) were captured onto IgG-specific BLI sensors with a loading level of approx. 2 nm. For analysis, human CD3 epsilon antigen hCD3e(22-118)_F-chLys_avi (SEQ ID NO: 43) was diluted with assay buffer to concentrations ranging from 200 nM to 3.1 nM. A blank sample with assay buffer was included for referencing, i.e. correcting for dissociation of captured antibody. The association phase was recorded for 180 s, followed by a dissociation phase of 300 s. The sensorgrams were fitted using Octet Data Analysis Software 10.0 (FortéBio, Sartorius AG) to determine kₒₙ and k_{off} rate constants (using a 1:1 binding model), which were used to calculate K₀.

FIG. 10 summarizes K_{D} values for 24 preferred bispecific single point variant antibodies on human CD3epsilon antigen. These variants revealed monovalent affinities in the single digit nanomolar range comparable to that of parental antibody CD3-MAB_{opt-cc}.

### Example 4.5: Summary for Deimmunized Single Point Variant of CD3-MAB_{opt-cc}

FIG. 10 summarizes the biophysical and functional characteristics of 24 preferred bispecific single point variant antibodies (5 LCDR3 variants and 19 HCDR variants) which were selected based on their producibility and ELISA binding. All variants revealed a reduced risk for immunogenicity as determined by a reduction of T cell epitopes, H-lines, Absolute Score and H-Score but retained the biophysical and functional characteristics of the parental CD3-MAB_{opt-cc} when tested in the bispecific antibody format as described herein.

14 out of the 19 single point variants (as listed in Table 32) were selected of a combinatorial T-cell epitope Epibase™ analysis as described below (see Example 4.6) because a reduction of Hotspots could be only determined for 5 out of the 19 single point variants. Table 32 summarizes the impact of each of the selected 14 single point variants on the immunogenicity risk parameters compared to the parameters for the parental antibody. Notably, only substitutions in HCDR2 position V61 and in HCDR1 position Y33 led to a reduction of Hotspots.

For the VL of CD3-MAB_{opt-cc}, the LCDR3 variant S95E was selected as the only preferred VL variant to be used in connection with any other VH variant. This single point LCDR3 variant resulted in a reduction of 4 H-lines in the LCDR3 and revealed similar functional and biophysical characteristics as the parental antibody VH of CD3-MAB_{opt-cc}, when tested in the bispecific antibody format of Example 4.2.

TABLE 32
Summary Epibase ™ T cell epitope screening for the VH
of CD3-MAB_{opt-cc} for preferred HCDR single-point variants

| | | EpiBase ™ Screen | | | | | | | |
|---|---|---|---|---|---|---|---|---|---|
| | | | Δ | | Δ | | Δ | | Δ |
| HCDR | Mutated | Absolut | Absolut | Hot- | Hot- | Absolute | Absolute | Absolut | Absolut |
| variant | region | Score | Score | spots | spots | H-score | H-score | H-lines | H-lines |
| wt (CD3- | n.a | 653 | n.a | 6 | n.a. | 580 | n.a. | 25 | n.a. |
| MAB_{opt-cc}) | | | | | | | | | |
| F104A | HCDR3 | 610 | −43 | 6 | 0 | 556 | −24 | 23 | −2 |
| A105G | | 595 | −58 | 6 | 0 | 556 | −24 | 23 | −2 |
| H106G | | 626 | −27 | 6 | 0 | 556 | −24 | 23 | −2 |
| H106K | | 611 | −42 | 6 | 0 | 556 | −24 | 23 | −2 |
| H106T | | 626 | −27 | 6 | 0 | 556 | −24 | 23 | −2 |
| A102D | | 548 | −104 | 6 | 0 | 494 | −86 | 22 | −3 |
| A102E | | 578 | −75 | 6 | 0 | 524 | −56 | 22 | −3 |
| Y56S | HCDR2 | 643 | −9 | 6 | 0 | 571 | −9 | 20 | −5 |
| Y56T | | 599 | −54 | 6 | 0 | 526 | −54 | 20 | −5 |
| V61A | | 618 | −35 | 5 | −1 | 479 | −101 | 20 | −5 |
| V61D | | 497 | −155 | 4 | −2 | 421 | −159 | 17 | −8 |
| V61E | | 497 | −155 | 4 | −2 | 421 | −159 | 17 | −8 |
| V61G | | 512 | −140 | 4 | −2 | 421 | −159 | 17 | −8 |
| Y33W | HCDR1 | 607 | −45 | 5 | −1 | 490 | −90 | 21 | −4 |

### Example 4.6 Generation of Deimmunized Sequences by Combinatorial Amino Acid Substitution Variants (“Combined Variants”)

In order to further reduce the number of potential T cell epitopes and other immunogenicity risk parameters in the VH-CDR regions of CD3-MAB_{opt-cc}, an in silicio Epibase™ mutation analysis was performed wherein each of the previously identified 14 preferred HCDR single point variants of Table 32 were virtually combined with each other including the wildtype residues of CD3-MAB_{opt-cc} at each position. Accordingly, 251 new combinatorial VH-CDR sequence variants (incl. double, triple and quadruple mutations) were screened for potential T cell epitopes.

### Result of Epibase™ Screening Analysis for Combinatorial Mutation Variants

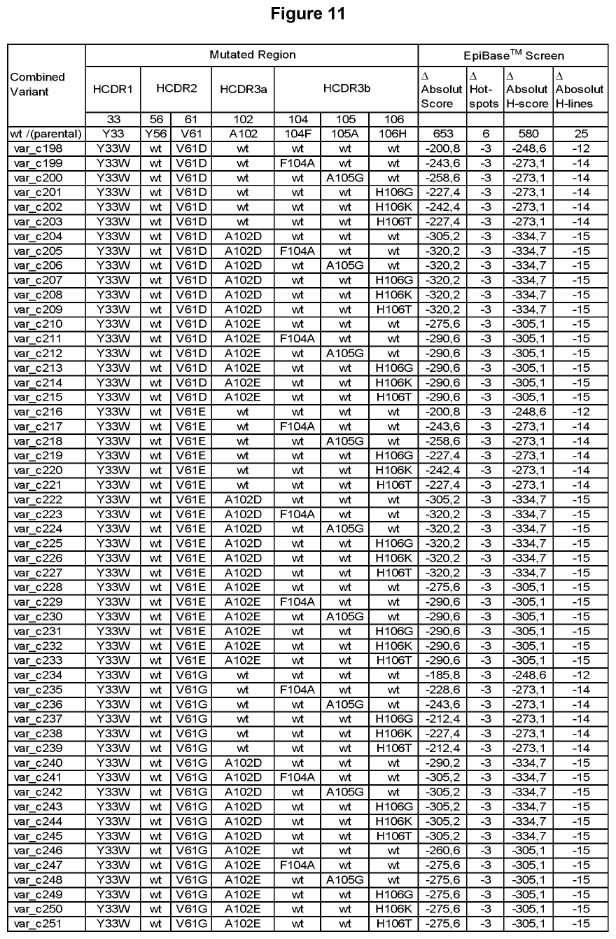

FIG. 11 depicts exemplary results of the impact of 54 combinatorial amino acid substitution in the VH_HCDR1-3 regions of CD3-MAB_{opt-cc} on the different immunogenicity risk parameters which resulted in a maximum reduction of 3 Hotspots. The first row of the table indicates the risk parameters: Absolut Score, Hotspots, Absolut H-Score and Absolut H-lines) for the VH of CD3-MAB_{opt-cc} (denoted as “wt (parental)).

Surprisingly, crucial combined variants for reduction of Hotspots, Absolut H-lines and Absolut H-Score could be allocated to the mutant combination of Y33W (HCDR1)+wt Y56 (HCDR2)+V61D/E/ or G (HCDR2).

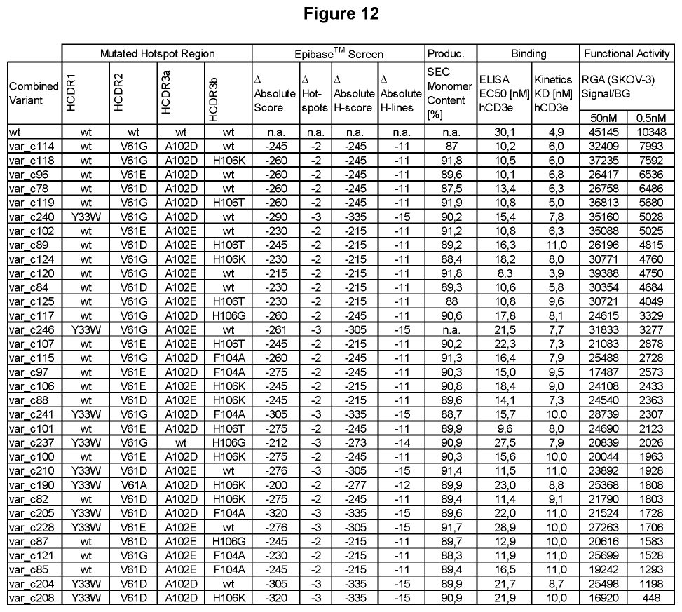

All 54 VH combined variants which resulted in a reduction of 3 Hotspots and 40 additional variant combinations which resulted in a reduction of 2 Hotspots (not shown) and revealed the strongest reduction of H-lines and Absolute Score were selected for cloning and production in the bispecific antibody format as described in Example 3. For this, each of the 54 combined VH variants were expressed together with the VL variant comprising the LCDR3 variant S95E (SEQ ID NO: 21). 25/94 produced bispecific combined variant antibodies revealed a monomer content of >85%. FIG. 12 summarizes monomer content of 33 preferred bispecific combined variant antibody preparations as determined by analytical size exclusion chromatography.

### Example 4.7: ELISA Binding of Bispecific Antibodies to Recombinant Human CD3epsilon

All 94 produced bispecific combined variant antibodies of Example 4.6 were tested for ELISA binding to recombinant CD3epsilon antigen as previously described in Example 4.3. In sum, 46 bispecific combined variant antibodies revealed better binding to CD3epsilon when compared to the parental antibody CD3-MAB_{opt-cc} with EC₅₀ values of <30 nM. FIG. 12 summarizes ELISA EC₅₀ values for 33 preferred bispecific combined variants determined on human CD3epsilon antigen.

### Example 4.8: K_{D} Determination Via Antibody Capture Setup

Affinity determination by determining kinetic rate constants for 46 bispecific combined variants of Example 4.6 which revealed better ELISA binding to CD3 (Example 4.7) was done as previously described in Example 4.4. Overall, bispecific combined variant antibodies revealed monovalent affinities on CD3epsilon in the mid-single to low-double digit nanomolar range. No improved binding to CD3 over the bispecific antibody comprising the parental antibody CD3-MAB_{opt-cc} (K_{D}=5 nM) could be achieved. FIG. 12 summarizes K_{D} values for 33 preferred bispecific combined variant antibodies on human CD3epsilon antigens. These variants revealed K_{D} values in the range of 5-11 nM, comparable to the affinity determined for the corresponding bispecific antibody comprising the parental antibody CD3-MAB_{opt-cc}.

### Example 4.9 Jurkat NFAT Reporter Gene Cell Assay

A Jurkat NFAT Reporter Gene Cell assay was conducted to evaluate the functional activity of 46 bispecific combined variant antibodies of Example 4.8 with specificity for HER2 and CD3. Jurkat cells (ATCC #TIB-152) stably transfected with an NFAT reporter gene construct were used as surrogate effector cells. As tumor target cells, the HER2 positive human SKOV-3 (ATCC® HTB-77) tumor cell line was used. The assay was conducted as described before in Example 2.3. Bispecific antibodies were serially diluted to a final assay concentration range of 50 nM to 0.5 nM (4 step dilution).

Most bispecific combined variant antibodies induced luciferase activity in the presence of SKOV-3 cells. However, none of the tested bispecific antibodies revealed superior activity in terms of EC₅₀ value and maximum luciferase activity levels over the bispecific antibody comprising the parental antibody CD3-MAB_{opt-cc}.

FIG. 12 summarizes the RGA results for 33 preferred bispecific combined variant antibodies. Results are provided as signal over background values at two tested antibody concentration and antibodies were sorted accordingly. The top row of the table provides RGA results for the parental bispecific antibody CD3-MAB_{opt-cc} indicating highest signal intensities for this antibody at an antibody conc. of 50 nM and 0.5 nM. Overall, a positive correlation between activity in the RGA and binding affinity to CD3 can be suspected.

### Example 4.10 Exploratory Scale Production

33 preferred bispecific combined variants of Example 4.9 were selected for larger scale production. Selection criteria included K_{D} values on human CD3e (Example 4.8), functional activity in the reporter gene assay (Example 4.9) and monomer content after smaller scale production (Example 4.6).

Eukaryotic HEK293-6E cells were transfected with mammalian expression vector DNA encoding both heavy and light chains of the bispecific antibodies. Cell culture supernatants were harvested on day 6 post transfection and subjected to standard Protein A affinity chromatography (MabSelect SURE|GE Healthcare). Buffer exchange was performed to 1× Dulbcecco's PBS (pH 7.2|Invitrogen) and samples were sterile filtered (0.2 μm pore size). Protein concentrations and purities of bispecific antibodies were determined as previously described.

FIG. 12 summarizes the percentage of monomer content for the 33 produced bispecific combined variant antibody preparations. 15 of these preparations revealed a monomer antibody content of greater 90%.

### Example 4.11: Selection of the Most Appropriate Combined Variant CD3 Specific Antibodies

Parallel deimmunization of the LCDR3 region and HCDR1-3 regions of the CD3 specific antibody CD3-MABopt-cc, resulted in the identification of 5 preferred deimmunized VH/VL variants. These variants appeared amongst the 10 best performing bispecific antibodies in each of the aforementioned assays of Example 4. and were thus further characterized in T cell mediated cytotoxicity assays (Example 4.12) and their potential to induce T cell activation in the absence of target cells (Example 4.13).

The aforementioned 5 deimmunized CD3 specific antibodies are denoted herein: CD3-MABdeimm_1, CD3-MABdeimm_2, CD3-MABdeimm_3, CD3-MABdeimm_4, and CD3-MABdeimm_5. VH, VL and CDR sequences for each of the 5 deimmunized antibodies are provided in Table 7. The corresponding bispecific antibodies comprising the 5 deimmunized antibodies are denoted herein as: BissIg_21 #CD3-MABdeimm_1, BissIg_21 #CD3-MABdeimm_2, BissIg_21 #CD3-MABdeimm_3, BissIg_21 #CD3-MABdeimm_4 and BissIg_21 #CD3-MABdeimm_5, respectively. A summary of the individual polypeptides forming the bispecific antibodies made in accordance with Example 4 are set forth in Table 14. Table 29 provides a summary of the biophysical and functional characteristics as well as of the immunogenicity risk parameters for these 5 deimmunized CD3 specific antibodies and corresponding bispecific antibodies.

### Example 4.12: Re-Directed T Cell Cytotoxicity Mediated by Bispecific Antibodies Comprising 5 Preferred Deimmunized CD3 Specific Antibodies

Bispecific combined variant antibodies BissIg_21 #CD3-MABdeimm_1, BissIg_21 #CD3-MABdeimm_2, BissIg_21 #CD3-MABdeimm_3, BissIg_21 #CD3-MABdeimm_4 and BissIg_21 #CD3-MABdeimm_5 were tested for their potential to induce T cell mediated killing of tumor cells upon binding to CD3 and HER2. The method was carried out as described in Example 3.6. As target cells, 5,000 HER2 expressing SKOV-3 cells were used resulting in a target/effector cell ration of 1:10.

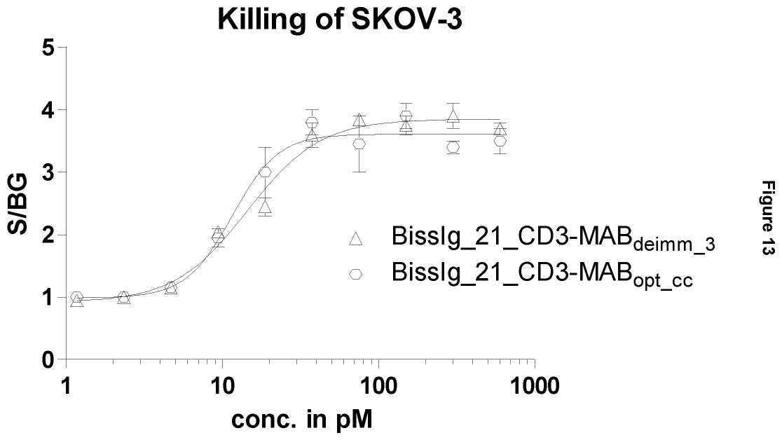

Table 33 summarizes IC₅₀ value of T cell mediated killing by all 5 tested bispecific combined variant antibodies. Concentration dependent killing of HER2 high expressing SKOV-3 cells could be confirmed for all 5 bispecific antibodies comparable to that of the bispecific antibody comprising the parental antibody CD3-MABopt-cc. FIG. 13 depicts exemplary SKOV-3 killing results for T-cell obtained from one doner and the most preferred combined variant antibody CD3-MABdeimm_3 (BissIg_21 #CD3-MABdeimm_3) in reference to the parental antibody CD3-MABopt-cc (BissIg_21 #CD3-MABopt-cc).

### Example 4.13: T Cell Activation of Bispecific Antibodies in the Absence of Target Cancer Cells (High-Density PBMC Assay)

To evaluate the safety of the deimmunized CD3 specific antibodies, the 5 corresponding bispecific combined variant antibodies, BissIg_21 #CD3-MABdeimm_1 (CD3-MABdeimm_1), BissIg_21 #CD3-MABdeimm_2 (CD3-MABdeimm_2), BissIg_21 #CD3-MABdeimm_3 (CD3-MABdeimm_3), BissIg_21 #CD3-MABdeimm_4 (CD3-MABdeimm_4) and BissIg_21 #CD3-MABdeimm_5 (CD3-MABdeimm_5) were tested for their ability to activate human T cells in the absence of target cancer cells. The assay as carried out as previously under Example 3.7. Human blood samples were retrieved from two different donors. Activation of T cells was assessed by evaluation of upregulation of CD69 expression on CD4 positive or CD8 positive T cells.

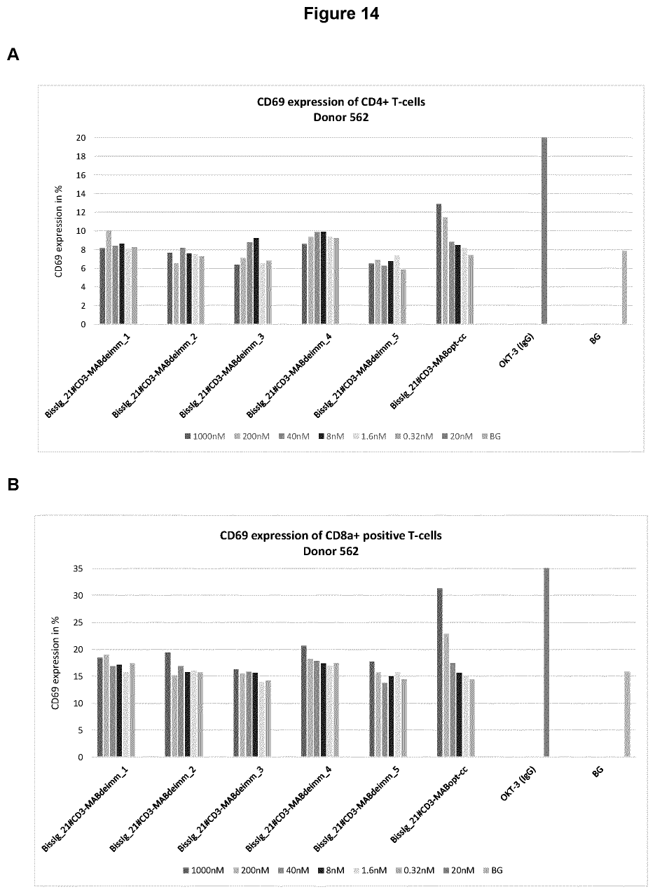

Table 33 qualitatively summarizes the T cell activation potential of the 5 bispecific combined variant antibodies. No activation of T cells was observable for any of the bispecific combined variant antibodies in the absence of HER2 expressing target cells even at the highest tested antibody concentration. A donor dependent activation of T cells could be observed for the bispecific antibody BissIg_21 #CD3-MABopt-cc comprising the parental antibody CD3-MABopt-cc at higher tested concentration. Positive control OKT-3 strongly induced CD69 expression of both, CD4+ and CD8+ T cells in the range of 76-78% at a IgG conc. of 20 nM. Exemplary results of the T cell activation experiments for one donor are shown in FIG. 14. FIG. 14A depicts the results for activation of CD4+/CD8− T cells and FIG. 14B shows the results for activation of CD4−/CD8+ T cells. These results clearly demonstrates the remarkable safety profile of the deimmunized human CD3 specific antibodies of the present disclosure.

### Example 4.14: Summary for Deimmunized Combined Variants

The present invention provides for the first time, a CDR deimmunization approach for fully human antibodies with specificity for CD3. An overview of the functional and biophysical characteristics of the 5 preferred deimmunized variants CD3-MABdeimm_1, CD3-MABdeimm_2, CD3-MABdeimm_3, CD3-MABdeimm_4, and CD3-MABdeimm_5 and their parental human CD3 specific antibody CD3-MABopt-cc identified in Example 2 is provided in Table 33. CD3-MAB_{deimmun_3} was identified as the most preferred deimmunized CD3 specific antibody of the present invention. CDR engineering of the parental antibody CD3-MABopt-cc resulted in a total reduction of 2 Hotspots in the HCDR2 region and 1 Hotspot in the LCDR3 region of this antibody. In addition, a significant reduction of T cell epitopes, H-Lines, Absolut Risk Score and Absolut H-Score could be achieved. Accordingly, CD3-MAB_{deimmun_3} has a significant reduced risk in inducing an immunogenic reaction in humans once administered, a very crucial aspect in T cell engaging therapies. This remarkable safety profile of CD3-MAB_{deimmun_3} was further strengthened by its inability to induce T cell activation in the absence of target cell. Noteworthy, CDR engineering did not result in a loss of specificity, functional activity and producibility, when compared to the parental antibody CD3-MABopt-cc. This is even more remarkable, as the CDR engineering affected the HCDR3 region of CD3-MABopt-cc, which is the most relevant CDR for antigen recognition.

TABLE 33
Overview of the functional and biophysical characteristic of 5 preferred deimmunized combined variant
antibodies derived from the parental antibody CD3-MABopt-cc according to the present invention.

| | | Anti-CD3 antibody | | | | | | | |
|---|---|---|---|---|---|---|---|---|---|
| | | | SEQ | SEQ | | | | | |
| Bispecific antibody | | Deimmunized | ID | ID | | | | | |
| Combined | SEQ ID | anti-CD3 | NO: | NO: | Mutated CDR3 position | | | | |
| Variant | Nos | antibody ID | VH | VL | HCDR1 | HCDR2 | HCDR3a | HCDR3b | LCDR3 |
| Bisslg_21#CD3- | 66, 67, 51 | CD3-MABopt_cc | 14 | 20 | wt | wt | wt | wt | wt |
| MABopt-cc | | | | | | | | | |
| Bisslg_21#CD3- | 70, 71, 51 | CD3-MAB_{deimm}_{_{—}}₁ | 18 | 21 | wt | V61E | A102E | wt | S95E |
| MABdeimm_1 | | | | | | | | | |
| Bisslg_21#CD3- | 70, 72, 51 | CD3-MAB_{deimm}_{_{—}}₂ | 19 | 21 | wt | V61G | A102D | wt | S95E |
| MABdeimm_2 | | | | | | | | | |
| Bisslg_21#CD3- | 70, 73, 51 | CD3-MAB_{deimm}_{_{—}}₃ | 15 | 21 | wt | V61G | A102D | H106T | S95E |
| MABdeimm_3 | | | | | | | | | |
| Bisslg_21#CD3- | 70, 74, 51 | CD3-MAB_{deimm}_{_{—}}₄ | 16 | 21 | wt | V61G | A102E | wt | S95E |
| MABdeimm_4 | | | | | | | | | |
| Bisslg_21#CD3- | 70, 75, 51 | CD3-MAB_{deimm}_{_{—}}₅ | 17 | 21 | wt | V61G | A102D | H106K | S95E |
| MABdeimm_5 | | | | | | | | | |

| | Anti-CD3 | | | | | | | | |
|---|---|---|---|---|---|---|---|---|---|
| | antibody | | | | | | | | |
| Bispecific antibody | Deimmunized | Epibase ™ Risk Analysis VH | | | | | | | |
| Combined | anti-CD3 | Absolute | Δ | Absolute | Δ | Absolute | Δ | Absolute | Δ |
| Variant | antibody ID | Score | Score | Hotspots | Hotspots | H-score | H-score | H-lines | H-lines |
| Bisslg_21#CD3- | CD3-MABopt_cc | 653 | — | 6 | — | 580 | — | 25 | — |
| MABopt-cc | | | | | | | | | |
| Bisslg_21#CD3- | CD3-MAB_{deimm}_{_{—}}₁ | 422 | −230 | 4 | −2 | 365 | −215 | 14 | −11 |
| MABdeimm_1 | | | | | | | | | |
| Bisslg_21#CD3- | CD3-MAB_{deimm}_{_{—}}₂ | 408 | −245 | 4 | −2 | 335 | −245 | 14 | −11 |
| MABdeimm_2 | | | | | | | | | |
| Bisslg_21#CD3- | CD3-MAB_{deimm}_{_{—}}₃ | 393 | −260 | 4 | −2 | 335 | −245 | 14 | −11 |
| MABdeimm_3 | | | | | | | | | |
| Bisslg_21#CD3- | CD3-MAB_{deimm}_{_{—}}₄ | 437 | −215 | 4 | −2 | 365 | −215 | 14 | −11 |
| MABdeimm_4 | | | | | | | | | |
| Bisslg_21#CD3- | CD3-MAB_{deimm}_{_{—}}₅ | 393 | −260 | 4 | −2 | 335 | −245 | 14 | −11 |
| MABdeimm_5 | | | | | | | | | |

| Bispecific | | | | | | | | |
|---|---|---|---|---|---|---|---|---|
| antibody | Epibase ™ Risk Analysis for VL | | | | | | | |
| Combined | Absolute | Δ | Absolute | Δ | Absolute | Δ | Absolute | Δ |
| Variant | Score | Score | Hotspots | Hotspots | H-score | H-score | H-lines | H-lines |
| Bisslg_21#CD3- | 189 | — | 1 | — | 189 | — | 7 | — |
| MABopt-cc | | | | | | | | |
| Bisslg_21#CD3- | 62 | 127 | 0 | −1 | 62 | 127 | 2 | −4 |
| MABdeimm_1 | | | | | | | | |
| Bisslg_21#CD3- | | | | | | | | |
| MABdeimm_2 | | | | | | | | |
| Bisslg_21#CD3- | | | | | | | | |
| MABdeimm_3 | | | | | | | | |
| Bisslg_21#CD3- | | | | | | | | |
| MABdeimm_4 | | | | | | | | |
| Bisslg_21#CD3- | | | | | | | | |
| MABdeimm_5 | | | | | | | | |

| | Anti-CD3 | | | | |
|---|---|---|---|---|---|
| Bispecific | antibody | Production | | Binding | |
| antibody | Deimmunized | | Volumetric | ELISA | |
| Combined | anti-CD3 | SEC Monomer | yield | on hCD3 | KD [nM] |
| Variant | antibody ID | Content [%] | [mg/L] | EC50 [nM] | on hCD3e |
| Bisslg_21#CD3- | CD3-MABopt_cc | n.a | n.a. | 30.1 | 4.9 |
| MABopt-cc | | | | | |
| Bisslg_21#CD3- | CD3-MAB_{deimm}_{_{—}}₁ | 91.2 | 78.9 | 10.8 | 6.3 |
| MABdeimm_1 | | | | | |
| Bisslg_21#CD3- | CD3-MAB_{deimm}_{_{—}}₂ | 87.0 | 90.3 | 10.2 | 6.0 |
| MABdeimm_2 | | | | | |
| Bisslg_21#CD3- | CD3-MAB_{deimm}_{_{—}}₃ | 91.9 | 86.0 | 10.8 | 5.0 |
| MABdeimm_3 | | | | | |
| Bisslg_21#CD3- | CD3-MAB_{deimm}_{_{—}}₄ | 91.8 | 73.6 | 8.3 | 3.9 |
| MABdeimm_4 | | | | | |
| Bisslg_21#CD3- | CD3-MAB_{deimm}_{_{—}}₅ | 91.8 | 74.3 | 10.5 | 6.0 |
| MABdeimm_5 | | | | | |

| | | | | T-cell | |
|---|---|---|---|---|---|
| Bispecific | RGA | | T-cell killing | activation | |
| antibody | SKOV-3 | | SKOV-3 | CD4+ | CD8+ |
| Combined | Signal-BG | Signal-BG | IC50 [pM] | T-cells/CD64 | T-cells/CD64 |
| Variant | at 0.5 nM | at 50 nM | (n = 2) | upregulation | upregulation |
| Bisslg_21#CD3- | 10348 | 45145 | 9.8 | yes | yes |
| MABopt-cc | | | | | |
| Bisslg_21#CD3- | 5025 | 35088 | 17.9 | no | no |
| MABdeimm_1 | | | | | |
| Bisslg_21#CD3- | 7993 | 32409 | 10.5 | no | no |
| MABdeimm_2 | | | | | |
| Bisslg_21#CD3- | 5680 | 36813 | 13.1 | no | no |
| MABdeimm_3 | | | | | |
| Bisslg_21#CD3- | 4750 | 39388 | 10.9 | no | no |
| MABdeimm_4 | | | | | |
| Bisslg_21#CD3- | 7592 | 37235 | 19.0 | no | no |
| MABdeimm_5 | | | | | |

1: An isolated human antibody or antigen-binding fragment thereof specific for cluster of differentiation 3 (CD3), comprising
i. a heavy chain variable region (VH) comprising
(a) a heavy chain complementary determining region (HCDR)1 comprising the amino acid sequence of GFSFGSHYMS (SEQ ID NO: 1),
(b) a HCDR2 comprising the amino acid sequence of NINQIGYSSYYVESVKG (SEQ ID NO: 2), NINQIGYSSYYGESVKG (SEQ ID NO: 3) or NINQIGYSSYYEESVKG (SEQ ID NO: 4), and
(c) a HCDR3 comprising the amino acid sequence of GYSAEFAHRSGLDV (SEQ ID NO: 5), GYSDEFATRSGLDV (SEQ ID NO: 6), GYSEEFAHRSGLDV (SEQ ID NO: 7), GYSDEFAKRSGLDV (SEQ ID NO: 8) or GYSDEFAHRSGLDV (SEQ ID NO: 9), and
ii. a variable light chain region (VL) comprising
(a) a light chain complementary determining region (LCDR)1 comprising the amino acid sequence of SGSSSNIGSNYVY (SEQ ID NO: 10),
(b) a LCDR2 comprising the amino acid sequence of RNNQRPS (SEQ ID NO: 11), and
(c) a LCDR3 comprising the amino acid sequence of AGWSRSLHGAV (SEQ ID NO: 12) or AGWSRELHGAV (SEQ ID NO: 13).

2: The antibody or antigen-binding fragment thereof according to claim 1,
wherein said antibody or antigen-binding fragment thereof cross-reactively binds to cynomolgus CD3.

3: The antibody or antigen-binding fragment thereof according to claim 1, wherein said antibody or antigen-binding fragment thereof is a deimmunized antibody.

4: The antibody or antigen-binding fragment thereof according to claim 1, wherein said antibody or antigen-binding fragment thereof has a reduced risk in electing an immune response in human beings.

5: The antibody or antigen-binding fragment thereof according to claim 1, wherein the VH comprises an amino acid sequence that is at least about 95%, 96%, 97%, 98%, 99% or 100% identical to an amino acid sequence selected from the group consisting of SEQ ID NO: 14, SEQ ID NO: 15, SEQ ID NO: 16, SEQ ID NO: 17, SEQ ID NO: 18 and SEQ ID NO: 19, and/or the VL comprises an amino acid sequence that is at least about 95%, 96%, 97%, 98%, 99% or 100% identical to the amino acid sequence of SEQ ID NO: 20 or SEQ ID NO: 21.

6: The antibody or antigen-binding fragment thereof according to claim 1, wherein the VH and VL are selected from the group consisting of:
i. the VH comprising the amino acid sequence of SEQ ID NO: 14 and the VL comprising the amino acid sequence of SEQ ID NO: 20,
ii. the VH comprising the amino acid sequence of SEQ ID NO: 15 and the VL comprising the amino acid sequence of SEQ ID NO: 21,
iii. the VH comprising the amino acid sequence of SEQ ID NO: 16 and the VL comprising the amino acid sequence of SEQ ID NO: 21,
iv. the VH comprising the amino acid sequence of SEQ ID NO: 17 and the VL comprising the amino acid sequence of SEQ ID NO: 21,
v. the VH comprising the amino acid sequence of SEQ ID NO: 18 and the VL comprising the amino acid sequence of SEQ ID NO: 21, and
vi. the VH comprising the amino acid sequence of SEQ ID NO: 19 and the VL comprising the amino acid sequence of SEQ ID NO: 21.

7: The antibody or antigen-binding fragment thereof according to claim 1, wherein said antibody or antigen-binding fragment thereof is a recombinant antibody or antigen-binding fragment thereof.

8: The antibody or antigen-binding fragment thereof according to claim 1, wherein said antibody or antigen-binding fragment thereof is a monoclonal antibody or antigen-binding fragment thereof.

9: The antibody or antigen-binding fragment thereof according to claim 1, wherein the antigen-binding fragment is a Fab, Fab′, (Fab′)₂, Fv, or scFv.

10: The antibody or antigen-binding fragment thereof according to claim 1, wherein the antibody is a full-length antibody.

11: A bispecific antibody comprising an antigen-binding fragment of an antibody according to claim 1 and a second antigen-binding fragment of an antibody which binds to a different target antigen than said first antigen binding fragment.

12: The bispecific antibody according to claim 11, wherein the second antigen-binding fragment binds to a cell surface antigen, in particular a tumor associated cell surface antigen.

13: The antibody or antigen-binding fragment thereof according to claim 1, comprising an Fe region, which comprises one or more amino acid substitution that reduces binding to an Fc receptor and/or effector function.

14: A nucleic acid composition comprising a nucleic acid sequence or a plurality of nucleic acid sequences encoding the antibody or antigen-binding fragment thereof according to claim 1.

15: A vector composition comprising a vector or a plurality of vectors comprising the nucleic acid sequence or plurality of nucleic acid sequences according to claim 14.

16: A host cell comprising the vector composition according to claim 15.

17: A pharmaceutical composition comprising the antibody or antigen-binding fragment thereof according to claim 1 and a pharmaceutically acceptable carrier or excipient.

18. (canceled)

19: A method for stimulating T cell activation, said method comprising contacting a T cell with the antibody or antigen-binding fragment thereof according to claim 1.

20: The method of claim 19 wherein the T cell expresses CD3.

21: The method of claim 19 wherein the T cell is in a subject with cancer.


SEQUENCE LISTING (104 sequences)

SEQ ID NO: 1 - protein, 10 aa, synthetic construct
GFSFGSHYMS

SEQ ID NO: 2 - protein, 17 aa, synthetic construct
NINQIGYSSYYVESVKG

SEQ ID NO: 3 - protein, 17 aa, synthetic construct
NINQIGYSSYYGESVKG

SEQ ID NO: 4 - protein, 17 aa, synthetic construct
NINQIGYSSYYEESVKG

SEQ ID NO: 5 - protein, 14 aa, synthetic construct
GYSAEFAHRSGLDV

SEQ ID NO: 6 - protein, 14 aa, synthetic construct
GYSDEFATRSGLDV

SEQ ID NO: 7 - protein, 14 aa, synthetic construct
GYSEEFAHRSGLDV

SEQ ID NO: 8 - protein, 14 aa, synthetic construct
GYSDEFAKRSGLDV

SEQ ID NO: 9 - protein, 14 aa, synthetic construct
GYSDEFAHRSGLDV

SEQ ID NO: 10 - protein, 13 aa, synthetic construct
SGSSSNIGSNYVY

SEQ ID NO: 11 - protein, 7 aa, synthetic construct
RNNQRPS

SEQ ID NO: 12 - protein, 11 aa, synthetic construct
AGWSRSLHGAV

SEQ ID NO: 13 - protein, 11 aa, synthetic construct
AGWSRELHGAV

SEQ ID NO: 14 - protein, 123 aa, synthetic construct
EVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSS

SEQ ID NO: 15 - protein, 123 aa, synthetic construct
EVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFATRSGLDVWGQGTLVTVSS

SEQ ID NO: 16 - protein, 123 aa, synthetic construct
EVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSEEFAHRSGLDVWGQGTLVTVSS

SEQ ID NO: 17 - protein, 123 aa, synthetic construct
EVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFAKRSGLDVWGQGTLVTVSS

SEQ ID NO: 18 - protein, 123 aa, synthetic construct
EVQLVESGGGLVQPGGSLRLSCAASGFSFGSHYMSWVRQAPGKGLEWVANINQIGYSSYYEESVKGRFTISRDNAKNSLYLQMNSLRAEDTAVYYCARGYSEEFAHRSGLDVWGQGTLVTVSS

SEQ ID NO: 19 - protein, 123 aa, synthetic construct
EVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFAHRSGLDVWGQGTLVTVSS

SEQ ID NO: 20 - protein, 112 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQ

SEQ ID NO: 21 - protein, 112 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAGWSRELHGAVFGGGTKLTVLGQ

SEQ ID NO: 22 - protein, 10 aa, synthetic construct
GFTFSSYWMS

SEQ ID NO: 23 - protein, 17 aa, synthetic construct
NIKQDGSEKYYVDSVKG

SEQ ID NO: 24 - protein, 11 aa, synthetic construct
AAWDHHRSGAV

SEQ ID NO: 25 - protein, 123 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGYSAEFAHRSGLDVWGQGTLVTVSS

SEQ ID NO: 26 - protein, 112 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDHHRSGAVFGGGTKLTVLGQ

SEQ ID NO: 27 - skipped

SEQ ID NO: 28 - protein, 120 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 29 - protein, 109 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 30 - protein, 9 aa, synthetic construct
GGSGGSGGS

SEQ ID NO: 31 - protein, 20 aa, synthetic construct
GGGGSGGGGSGGGGSGGGGS

SEQ ID NO: 32 - protein, 20 aa, synthetic construct
AHPAAPAPAHPAAPAPAHGH

SEQ ID NO: 33 - protein, 20 aa, synthetic construct
AQPAAPAPDAHEAPAPAQGS

SEQ ID NO: 34 - protein, 20 aa, synthetic construct
PKAAPSVTLFPPSSEELQAN

SEQ ID NO: 35 - protein, 20 aa, synthetic construct
ASTKGPSVFPLAPSSKSTSG

SEQ ID NO: 36 - protein, 5 aa, synthetic construct
PKAAP

SEQ ID NO: 37 - protein, 6 aa, synthetic construct
ASTKGP

SEQ ID NO: 38 - protein, 10 aa, synthetic construct
DKTHTCPPCP

SEQ ID NO: 39 - protein, 10 aa, synthetic construct
AQPAAPAPAE

SEQ ID NO: 40 - protein, 20 aa, synthetic construct
AQPAAPAPNAHEAPAPAQGS

SEQ ID NO: 41 - protein, 256 aa, synthetic construct
QDGNEEMGGITQTPYKVSISGTTVILTCDIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 256 aa, synthetic construct
QDGNEEMGSITQTPYQVSISGTTVILTCDIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 261 aa, synthetic construct
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVDIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 44 - protein, 252 aa, synthetic construct
QDGNEEMGSITQTPYQVSISGTTVILTCSQHLGSEAQWQHNGKNKEDSGDRLFLPEFSEMEQSGYYVCYPRGSNPEDASHHLYLKARVVIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 45 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 46 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 47 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 48 - protein, 177 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 49 - protein, 714 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAQPAAPAPAEQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQAQPAAPAPNAHEAPAPAQGSEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGYSAEFAHRSGLDVWGQGTLVTVSS

SEQ ID NO: 50 - protein, 450 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 714 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAQPAAPAPAEQSVLTQPPSASGTPGQRVTISCSGSSSNIGSNYVYWYQQLPGTAPKLLIYRNNQRPSGVPDRFSGSKSGTSASLAISGLRSEDEADYYCAAWDHHRSGAVFGGGTKLTVLGQAQPAAPAPNAHEAPAPAQGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSS

SEQ ID NO: 53 - protein, 450 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 54 - protein, 453 aa, synthetic construct
EVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 55 - protein, 216 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 576 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 588 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 587 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 599 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQAQPAAPAPDAHEAPAPAQGSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSAQPAAPAPDAHEAPAPAQGSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPSVTLFPPSSEELQANDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAHPAAPAPAHPAAPAPAHGHQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPSVTLFPPSSEELQANDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAHPAAPAPAHPAAPAPAHGHEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPSVTLFPPSSEELQANDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 591 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRSLHGAVFGGGTKLTVLGQPKAAPSVTLFPPSSEELQANDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLVESGGGLVQPGGSLRLSCAASGFSFGSHYMSWVRQAPGKGLEWVANINQIGYSSYYVESVKGRFTISRDNAKNSLYLQMNSLRAEDTAVYYCARGYSAEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - protein, 602 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSQSVLTQPPSASGTPGQRVTISCSGSSSNIGSNYVYWYQQLPGTAPKLLIYRNNQRPSGVPDRFSGSKSGTSASLAISGLRSEDEADYYCAGWSRELHGAVFGGGTKLTVLGQPKAAPSVTLFPPSSEELQANDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYEESVKGRFTISRDNAKNSLYLQMNSLRAEDTAVYYCARGYSEEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFATRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSEEFAHRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 613 aa, synthetic construct
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCAQPAAPAPDAHEAPAPAQGSEVQLVESGGGLVQPGGSLRLSCAASGFSFGSHYMSWVRQAPGKGLEWVANINQIGYSSYYGESVKGRFTISRDNAKNSLYLQMNSLRAEDTAVYYCARGYSDEFAKRSGLDVWGQGTLVTVSSASTKGPSVFPLAPSSKSTSGDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 15 aa, synthetic construct
PKAAPDKTHTCPPCP

SEQ ID NO: 77 - protein, 16 aa, synthetic construct
ASTKGPDKTHTCPPCP

SEQ ID NO: 78 - protein, 30 aa, synthetic construct
AQPAAPAPDAHEAPAPAQGSDKTHTCPPCP

SEQ ID NO: 79 - protein, 30 aa, synthetic construct
PKAAPSVTLFPPSSEELQANDKTHTCPPCP

SEQ ID NO: 80 - protein, 30 aa, synthetic construct
ASTKGPSVFPLAPSSKSTSGDKTHTCPPCP

SEQ ID NO: 81 - protein, 10 aa, synthetic construct
DTAVYYCARG

SEQ ID NO: 82 - protein, 10 aa, synthetic construct
TAVYYCARGY

SEQ ID NO: 83 - protein, 10 aa, synthetic construct
AVYYCARGYS

SEQ ID NO: 84 - protein, 10 aa, synthetic construct
VYYCARGYSA

SEQ ID NO: 85 - protein, 10 aa, synthetic construct
YYCARGYSAE

SEQ ID NO: 86 - protein, 10 aa, synthetic construct
CARGYSAEFA

SEQ ID NO: 87 - protein, 10 aa, synthetic construct
ARGYSAEFAH

SEQ ID NO: 88 - protein, 10 aa, synthetic construct
RGYSAEFAHR

SEQ ID NO: 89 - protein, 10 aa, synthetic construct
GYSAEFAHRS

SEQ ID NO: 90 - protein, 10 aa, synthetic construct
YSAEFAHRSG

SEQ ID NO: 91 - protein, 10 aa, synthetic construct
SAEFAHRSGL

SEQ ID NO: 92 - protein, 10 aa, synthetic construct
AEFAHRSGLD

SEQ ID NO: 93 - protein, 10 aa, synthetic construct
EFAHRSGLDV

SEQ ID NO: 94 - protein, 10 aa, synthetic construct
FAHRSGLDVW

SEQ ID NO: 95 - protein, 10 aa, synthetic construct
AHRSGLDVWG

SEQ ID NO: 96 - protein, 10 aa, synthetic construct
HRSGLDVWGQ

SEQ ID NO: 97 - protein, 10 aa, synthetic construct
RSGLDVWGQG

SEQ ID NO: 98 - protein, 10 aa, synthetic construct
SGLDVWGQGT

SEQ ID NO: 99 - protein, 10 aa, synthetic construct
GLDVWGQGTL

SEQ ID NO: 100 - protein, 10 aa, synthetic construct
LDVWGQGTLV

SEQ ID NO: 101 - protein, 10 aa, synthetic construct
DVWGQGTLVT

SEQ ID NO: 102 - protein, 10 aa, synthetic construct
VWGQGTLVTV

SEQ ID NO: 103 - protein, 21 aa, synthetic construct
AVYYCARGYSAEFAHRSGLDV

SEQ ID NO: 104 - protein, 10 aa, synthetic construct
YCARGYSAEF